SEQUENCE LISTING (165 sequences)

SEQ ID NO: 1 - DNA, 28 bp, synthetic construct
  misc_feature 1..28: note = oligonucleotide
  source 1..28: mol_type = other DNA
tgtggggccctgaaaggcgagctgagat

SEQ ID NO: 2 - DNA, 28 bp, synthetic construct
  misc_feature 1..28: note = oligonucleotide
  source 1..28: mol_type = other DNA
atgggccgccatacgacgacgctcaact

SEQ ID NO: 3 - DNA, 24 bp, synthetic construct
  misc_feature 1..24: note = oligonucleotide
  source 1..24: mol_type = other DNA
gaagtctggttccttggcaggatg

SEQ ID NO: 4 - DNA, 20 bp, synthetic construct
  misc_feature 1..20: note = oligonucleotide
  source 1..20: mol_type = other DNA
actcgatacactggcctagc

SEQ ID NO: 5 - DNA, 20 bp, synthetic construct
  misc_feature 1..20: note = oligonucleotide
  source 1..20: mol_type = other DNA
caggtgtttgagggtagctc

SEQ ID NO: 6 - DNA, 20 bp, synthetic construct
  misc_feature 1..20: note = oligonucleotide
  source 1..20: mol_type = other DNA
cggttcatcatggtacagtc

SEQ ID NO: 7 - DNA, 24 bp, synthetic construct
  misc_feature 1..24: note = oligonucleotide
  source 1..24: mol_type = other DNA
actgcccctcatcagactgctact

SEQ ID NO: 8 - DNA, 24 bp, synthetic construct
  misc_feature 1..24: note = oligonucleotide
  source 1..24: mol_type = other DNA
cactgccttgtactcgggtagctg

SEQ ID NO: 9 - DNA, 24 bp, synthetic construct
  misc_feature 1..24: note = oligonucleotide
  source 1..24: mol_type = other DNA
gttccaacctgtgcctcgcgtctt

SEQ ID NO: 10 - DNA, 26 bp, synthetic construct
  misc_feature 1..26: note = oligonucleotide
  source 1..26: mol_type = other DNA
agcgaggcatggagagagcggagcag

SEQ ID NO: 11 - DNA, 23 bp, synthetic construct
  misc_feature 1..23: note = oligonucleotide
  source 1..23: mol_type = other DNA
cgtggtgagcatcttcggagtgg

SEQ ID NO: 12 - DNA, 23 bp, synthetic construct
  misc_feature 1..23: note = oligonucleotide
  source 1..23: mol_type = other DNA
ccttcttggtccgcccgttctta

SEQ ID NO: 13 - DNA, 30 bp, synthetic construct
  misc_feature 1..30: note = oligonucleotide
  source 1..30: mol_type = other DNA
atggacgcaactgtgaacatgatgttcgca

SEQ ID NO: 14 - DNA, 30 bp, synthetic construct
  misc_feature 1..30: note = oligonucleotide
  source 1..30: mol_type = other DNA
ctttgaggtcctggtccatcacgtgaccat

SEQ ID NO: 15 - DNA, 24 bp, synthetic construct
  misc_feature 1..24: note = oligonucleotide
  source 1..24: mol_type = other DNA
ccattaggggccatcatcgctttc

SEQ ID NO: 16 - DNA, 24 bp, synthetic construct
  misc_feature 1..24: note = oligonucleotide
  source 1..24: mol_type = other DNA
cactgctcactggagggggcttgc

SEQ ID NO: 17 - DNA, 24 bp, synthetic construct
  misc_feature 1..24: note = oligonucleotide
  source 1..24: mol_type = other DNA
tgctgcggtccaggccatcaagag

SEQ ID NO: 18 - DNA, 24 bp, synthetic construct
  misc_feature 1..24: note = oligonucleotide
  source 1..24: mol_type = other DNA
gggcactgttcagttcagcggatc

SEQ ID NO: 19 - DNA, 23 bp, synthetic construct
  misc_feature 1..23: note = oligonucleotide
  source 1..23: mol_type = other DNA
tctttccaccaggcccccggctc

SEQ ID NO: 20 - DNA, 23 bp, synthetic construct
  misc_feature 1..23: note = oligonucleotide
  source 1..23: mol_type = other DNA
tgcgggcggacatggggagatcc

SEQ ID NO: 21 - DNA, 30 bp, synthetic construct
  misc_feature 1..30: note = oligonucleotide
  source 1..30: mol_type = other DNA
attcttcgttgtcaagccgccaaagtggag

SEQ ID NO: 22 - DNA, 30 bp, synthetic construct
  misc_feature 1..30: note = oligonucleotide
  source 1..30: mol_type = other DNA
agttgtttgctgcggagttgtcatctcgtc

SEQ ID NO: 23 - DNA, 2127 bp, synthetic construct
  misc_feature 1..2127: note = oligonucleotide
  source 1..2127: mol_type = other DNA
atggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataa

SEQ ID NO: 24 - DNA, 456 bp, synthetic construct
  misc_feature 1..456: note = oligonucleotide
  source 1..456: mol_type = other DNA
atgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 25 - DNA, 297 bp, synthetic construct
  misc_feature 1..297: note = oligonucleotide
  source 1..297: mol_type = other DNA
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 26 - DNA, 23 bp, synthetic construct
  misc_feature 1..23: note = oligonucleotide
  source 1..23: mol_type = other DNA
ccccagggccccattttggtacc

SEQ ID NO: 27 - DNA, 26 bp, synthetic construct
  misc_feature 1..26: note = oligonucleotide
  source 1..26: mol_type = other DNA
ggcacccctggcatggctcttggctc

SEQ ID NO: 28 - DNA, 25 bp, synthetic construct
  misc_feature 1..25: note = oligonucleotide
  source 1..25: mol_type = other DNA
acgatcgtggccccggaaaaggacc

SEQ ID NO: 29 - DNA, 27 bp, synthetic construct
  misc_feature 1..27: note = oligonucleotide
  source 1..27: mol_type = other DNA
caacaaccgaaaatgcaccagccccag

SEQ ID NO: 30 - DNA, 24 bp, synthetic construct
  misc_feature 1..24: note = oligonucleotide
  source 1..24: mol_type = other DNA
ttatcgtcgaccactgtgctgctg

SEQ ID NO: 31 - DNA, 24 bp, synthetic construct
  misc_feature 1..24: note = oligonucleotide
  source 1..24: mol_type = other DNA
ccctttttctggagactaaataaa

SEQ ID NO: 32 - DNA, 24 bp, synthetic construct
  misc_feature 1..24: note = oligonucleotide
  source 1..24: mol_type = other DNA
gacagggggaggggaggagctagg

SEQ ID NO: 33 - DNA, 26 bp, synthetic construct
  misc_feature 1..26: note = oligonucleotide
  source 1..26: mol_type = other DNA
cttccctccaaccagttgccccaaac

SEQ ID NO: 34 - DNA, 26 bp, synthetic construct
  misc_feature 1..26: note = oligonucleotide
  source 1..26: mol_type = other DNA
gggaaatgggaggggtgcaaaagagg

SEQ ID NO: 35 - DNA, 26 bp, synthetic construct
  misc_feature 1..26: note = oligonucleotide
  source 1..26: mol_type = other DNA
ttgcgtgagtgtggatgggattggtg

SEQ ID NO: 36 - DNA, 25 bp, synthetic construct
  misc_feature 1..25: note = oligonucleotide
  source 1..25: mol_type = other DNA
cagccccgattcttccaccagtccc

SEQ ID NO: 37 - DNA, 25 bp, synthetic construct
  misc_feature 1..25: note = oligonucleotide
  source 1..25: mol_type = other DNA
cggaagattcccagtcgggttcacc

SEQ ID NO: 38 - DNA, 24 bp, synthetic construct
  misc_feature 1..24: note = oligonucleotide
  source 1..24: mol_type = other DNA
cttatgctacgtaaaggagctggg

SEQ ID NO: 39 - DNA, 24 bp, synthetic construct
  misc_feature 1..24: note = oligonucleotide
  source 1..24: mol_type = other DNA
gtgccaacccaggtcccggaagtt

SEQ ID NO: 40 - DNA, 24 bp, synthetic construct
  misc_feature 1..24: note = oligonucleotide
  source 1..24: mol_type = other DNA
cagatcctaaacagctcgcagaat

SEQ ID NO: 41 - DNA, 23 bp, synthetic construct
  misc_feature 1..23: note = oligonucleotide
  source 1..23: mol_type = other DNA
gcgtacgcaaattaaagtccaga

SEQ ID NO: 42 - DNA, 23 bp, synthetic construct
  misc_feature 1..23: note = oligonucleotide
  source 1..23: mol_type = other DNA
ctacaacgcctacgagtcctaca

SEQ ID NO: 43 - DNA, 24 bp, synthetic construct
  misc_feature 1..24: note = oligonucleotide
  source 1..24: mol_type = other DNA
gttgcaccagaaaagtcagagttg

SEQ ID NO: 44 - DNA, 24 bp, synthetic construct
  misc_feature 1..24: note = oligonucleotide
  source 1..24: mol_type = other DNA
atatcccgccgtgggtgaaagttc

SEQ ID NO: 45 - DNA, 24 bp, synthetic construct
  misc_feature 1..24: note = oligonucleotide
  source 1..24: mol_type = other DNA
actcagccatggactggagcatcc

SEQ ID NO: 46 - DNA, 24 bp, synthetic construct
  misc_feature 1..24: note = oligonucleotide
  source 1..24: mol_type = other DNA
ggagccgcctgccctggaaaattc

SEQ ID NO: 47 - DNA, 24 bp, synthetic construct
  misc_feature 1..24: note = oligonucleotide
  source 1..24: mol_type = other DNA
tttttcctgatattctattcccat

SEQ ID NO: 48 - DNA, 24 bp, synthetic construct
  misc_feature 1..24: note = oligonucleotide
  source 1..24: mol_type = other DNA
ccgtccccgcaatctccttccatc

SEQ ID NO: 49 - DNA, 24 bp, synthetic construct
  misc_feature 1..24: note = oligonucleotide
  source 1..24: mol_type = other DNA
atgatgccaacatggctcccggtg

SEQ ID NO: 50 - DNA, 26 bp, synthetic construct
  misc_feature 1..26: note = oligonucleotide
  source 1..26: mol_type = other DNA
cctgctcaagctgactcgacaccgtg

SEQ ID NO: 51 - DNA, 25 bp, synthetic construct
  misc_feature 1..25: note = oligonucleotide
  source 1..25: mol_type = other DNA
ggaaaagctggccctggggtggagc

SEQ ID NO: 52 - DNA, 28 bp, synthetic construct
  misc_feature 1..28: note = oligonucleotide
  source 1..28: mol_type = other DNA
tgattgtagtgctttctggctgggctcc

SEQ ID NO: 53 - DNA, 26 bp, synthetic construct
  misc_feature 1..26: note = oligonucleotide
  source 1..26: mol_type = other DNA
gcgtcctgggaagggagatccggagc

SEQ ID NO: 54 - DNA, 26 bp, synthetic construct
  misc_feature 1..26: note = oligonucleotide
  source 1..26: mol_type = other DNA
ttgaggggcatcgtcgcgggaggctg

SEQ ID NO: 55 - DNA, 24 bp, synthetic construct
  misc_feature 1..24: note = oligonucleotide
  source 1..24: mol_type = other DNA
cgagaggaccccgtggatgcagag

SEQ ID NO: 56 - DNA, 24 bp, synthetic construct
  misc_feature 1..24: note = oligonucleotide
  source 1..24: mol_type = other DNA
ggcggccatcttcagcttctccag

SEQ ID NO: 57 - DNA, 26 bp, synthetic construct
  misc_feature 1..26: note = oligonucleotide
  source 1..26: mol_type = other DNA
gccctctccctcccctccacgcacag

SEQ ID NO: 58 - DNA, 26 bp, synthetic construct
  misc_feature 1..26: note = oligonucleotide
  source 1..26: mol_type = other DNA
cggcgccgttgctcacagaccacagg

SEQ ID NO: 59 - DNA, 26 bp, synthetic construct
  misc_feature 1..26: note = oligonucleotide
  source 1..26: mol_type = other DNA
ggcccgccacttgcaggagtaccagg

SEQ ID NO: 60 - DNA, 26 bp, synthetic construct
  misc_feature 1..26: note = oligonucleotide
  source 1..26: mol_type = other DNA
cttctgctcgggcccctcatgagacg

SEQ ID NO: 61 - DNA, 28 bp, synthetic construct
  misc_feature 1..28: note = oligonucleotide
  source 1..28: mol_type = other DNA
acccattatccagatgtgtttgcccgag

SEQ ID NO: 62 - DNA, 26 bp, synthetic construct
  misc_feature 1..26: note = oligonucleotide
  source 1..26: mol_type = other DNA
atggtgaagctgggcataggcggcag

SEQ ID NO: 63 - DNA, 26 bp, synthetic construct
  misc_feature 1..26: note = oligonucleotide
  source 1..26: mol_type = other DNA
tgggagcggtgaagatggaagggcac

SEQ ID NO: 64 - DNA, 26 bp, synthetic construct
  misc_feature 1..26: note = oligonucleotide
  source 1..26: mol_type = other DNA
tcatgccagcgcccacgtacgacgac

SEQ ID NO: 65 - DNA, 26 bp, synthetic construct
  misc_feature 1..26: note = oligonucleotide
  source 1..26: mol_type = other DNA
cgctttcatggtgtgggctaaggacg

SEQ ID NO: 66 - DNA, 26 bp, synthetic construct
  misc_feature 1..26: note = oligonucleotide
  source 1..26: mol_type = other DNA
tagttggggtggtcctgcatgtgctg

SEQ ID NO: 67 - DNA, 26 bp, synthetic construct
  misc_feature 1..26: note = oligonucleotide
  source 1..26: mol_type = other DNA
cgccaggatccccgctttgaaatctg

SEQ ID NO: 68 - DNA, 26 bp, synthetic construct
  misc_feature 1..26: note = oligonucleotide
  source 1..26: mol_type = other DNA
tcggccgccagctctttgatgtgttc

SEQ ID NO: 69 - DNA, 24 bp, synthetic construct
  misc_feature 1..24: note = oligonucleotide
  source 1..24: mol_type = other DNA
ggaggcgtggagctcagcgacacc

SEQ ID NO: 70 - DNA, 24 bp, synthetic construct
  misc_feature 1..24: note = oligonucleotide
  source 1..24: mol_type = other DNA
cggggagctcgctgacggagtctg

SEQ ID NO: 71 - DNA, 30 bp, synthetic construct
  misc_feature 1..30: note = oligonucleotide
  source 1..30: mol_type = other DNA
caggtggcggacgtgtgaaaattgagagtg

SEQ ID NO: 72 - DNA, 26 bp, synthetic construct
  misc_feature 1..26: note = oligonucleotide
  source 1..26: mol_type = other DNA
cacgctggatctgcctggggactgtg

SEQ ID NO: 73 - DNA, 26 bp, synthetic construct
  misc_feature 1..26: note = oligonucleotide
  source 1..26: mol_type = other DNA
acagaggggaggtgcgccagttcacg

SEQ ID NO: 74 - DNA, 26 bp, synthetic construct
  misc_feature 1..26: note = oligonucleotide
  source 1..26: mol_type = other DNA
acggggtggacctcgctgcacagatc

SEQ ID NO: 75 - DNA, 26 bp, synthetic construct
  misc_feature 1..26: note = oligonucleotide
  source 1..26: mol_type = other DNA
ggcaccagcagcagcaggagcagcag

SEQ ID NO: 76 - DNA, 26 bp, synthetic construct
  misc_feature 1..26: note = oligonucleotide
  source 1..26: mol_type = other DNA
ccacgtctgaggagccgaggaagcag

SEQ ID NO: 77 - DNA, 26 bp, synthetic construct
  misc_feature 1..26: note = oligonucleotide
  source 1..26: mol_type = other DNA
gggccccatcaacttcaccgtcttcc

SEQ ID NO: 78 - DNA, 26 bp, synthetic construct
  misc_feature 1..26: note = oligonucleotide
  source 1..26: mol_type = other DNA
tgtagtcgatgttccccgccaggtcc

SEQ ID NO: 79 - DNA, 26 bp, synthetic construct
  misc_feature 1..26: note = oligonucleotide
  source 1..26: mol_type = other DNA
atgagcgggagaaggagcggcagaac

SEQ ID NO: 80 - DNA, 26 bp, synthetic construct
  misc_feature 1..26: note = oligonucleotide
  source 1..26: mol_type = other DNA
tcaatggccagcaccttcctcctctc

SEQ ID NO: 81 - DNA, 28 bp, synthetic construct
  misc_feature 1..28: note = oligonucleotide
  source 1..28: mol_type = other DNA
tttaacaccgccagcgctcttcaccttg

SEQ ID NO: 82 - DNA, 28 bp, synthetic construct
  misc_feature 1..28: note = oligonucleotide
  source 1..28: mol_type = other DNA
tcgtggcgcgtgtgttgtgggtatctcg

SEQ ID NO: 83 - DNA, 25 bp, synthetic construct
  misc_feature 1..25: note = oligonucleotide
  source 1..25: mol_type = other DNA
ctggaggccgagcagaagcgcaacg

SEQ ID NO: 84 - DNA, 25 bp, synthetic construct
  misc_feature 1..25: note = oligonucleotide
  source 1..25: mol_type = other DNA
gtccgcccgctcctctgcctcatcc

SEQ ID NO: 85 - DNA, 20 bp, synthetic construct
  misc_feature 1..20: note = oligonucleotide
  source 1..20: mol_type = other DNA
tttttttttttttttttttt

SEQ ID NO: 86 - DNA, 27 bp, synthetic construct
  misc_feature 1..27: note = oligonucleotide
  source 1..27: mol_type = other DNA
gccacagcaaacctcctcacagcccac

SEQ ID NO: 87 - DNA, 28 bp, synthetic construct
  misc_feature 1..28: note = oligonucleotide
  source 1..28: mol_type = other DNA
ctcgtcgtttccgcaacaagtcctcttc

SEQ ID NO: 88 - DNA, 26 bp, synthetic construct
  misc_feature 1..26: note = oligonucleotide
  source 1..26: mol_type = other DNA
caccatggcgggacacctggcttcag

SEQ ID NO: 89 - DNA, 26 bp, synthetic construct
  misc_feature 1..26: note = oligonucleotide
  source 1..26: mol_type = other DNA
acctcagtttgaatgcatgggagagc

SEQ ID NO: 90 - DNA, 31 bp, synthetic construct
  misc_feature 1..31: note = oligonucleotide
  source 1..31: mol_type = other DNA
caccatgtacaacatgatggagacggagctg

SEQ ID NO: 91 - DNA, 26 bp, synthetic construct
  misc_feature 1..26: note = oligonucleotide
  source 1..26: mol_type = other DNA
tcacatgtgtgagaggggcagtgtgc

SEQ ID NO: 92 - DNA, 30 bp, synthetic construct
  misc_feature 1..30: note = oligonucleotide
  source 1..30: mol_type = other DNA
caccatggctgtcagtgacgcgctgctccc

SEQ ID NO: 93 - DNA, 30 bp, synthetic construct
  misc_feature 1..30: note = oligonucleotide
  source 1..30: mol_type = other DNA
ttaaaaatgtctcttcatgtgtaaggcgag

SEQ ID NO: 94 - DNA, 30 bp, synthetic construct
  misc_feature 1..30: note = oligonucleotide
  source 1..30: mol_type = other DNA
caccatgcccctcaacgttagcttcaccaa

SEQ ID NO: 95 - DNA, 30 bp, synthetic construct
  misc_feature 1..30: note = oligonucleotide
  source 1..30: mol_type = other DNA
tcacgcacaagagttccgtagctgttcaag

SEQ ID NO: 96 - DNA, 27 bp, synthetic construct
  misc_feature 1..27: note = oligonucleotide
  source 1..27: mol_type = other DNA
caccatgggctgcaaaaacctgctcgg

SEQ ID NO: 97 - DNA, 27 bp, synthetic construct
  misc_feature 1..27: note = oligonucleotide
  source 1..27: mol_type = other DNA
tcatttgcactggtccaagttgctgtc

SEQ ID NO: 98 - DNA, 33 bp, synthetic construct
  misc_feature 1..33: note = oligonucleotide
  source 1..33: mol_type = other DNA
attgtcgacggggatttggcagggtcacaggac

SEQ ID NO: 99 - DNA, 32 bp, synthetic construct
  misc_feature 1..32: note = oligonucleotide
  source 1..32: mol_type = other DNA
cccagatctccaatgccacctcctcccaaacg

SEQ ID NO: 100 - DNA, 32 bp, synthetic construct
  misc_feature 1..32: note = oligonucleotide
  source 1..32: mol_type = other DNA
cactcgaggtggaggagctgagggcactgtgg

SEQ ID NO: 101 - DNA, 32 bp, synthetic construct
  misc_feature 1..32: note = oligonucleotide
  source 1..32: mol_type = other DNA
cacagatctgaaatgagggcttgcgaagggac

SEQ ID NO: 102 - DNA, 30 bp, synthetic construct
  misc_feature 1..30: note = oligonucleotide
  source 1..30: mol_type = other DNA
ggtttaaaagggtaaatgtgattatattta

SEQ ID NO: 103 - DNA, 22 bp, synthetic construct
  misc_feature 1..22: note = oligonucleotide
  source 1..22: mol_type = other DNA
caaactacaaccacccatcaac

SEQ ID NO: 104 - DNA, 30 bp, synthetic construct
  misc_feature 1..30: note = oligonucleotide
  source 1..30: mol_type = other DNA
gaggttggagtagaaggattgttttggttt

SEQ ID NO: 105 - DNA, 30 bp, synthetic construct
  misc_feature 1..30: note = oligonucleotide
  source 1..30: mol_type = other DNA
cccccctaacccatcacctccaccacctaa

SEQ ID NO: 106 - DNA, 25 bp, synthetic construct
  misc_feature 1..25: note = oligonucleotide
  source 1..25: mol_type = other DNA
tggttaggttggttttaaatttttg

SEQ ID NO: 107 - DNA, 26 bp, synthetic construct
  misc_feature 1..26: note = oligonucleotide
  source 1..26: mol_type = other DNA
aacccacccttataaattctcaatta

SEQ ID NO: 108 - DNA, 33 bp, synthetic construct
  misc_feature 1..33: note = oligonucleotide
  source 1..33: mol_type = other DNA
caccatgtacagcatgatgatggagaccgacct

SEQ ID NO: 109 - DNA, 26 bp, synthetic construct
  misc_feature 1..26: note = oligonucleotide
  source 1..26: mol_type = other DNA
ctagatatgcgtcaggggcaccgtgc

SEQ ID NO: 110 - DNA, 34 bp, synthetic construct
  misc_feature 1..34: note = oligonucleotide
  source 1..34: mol_type = other DNA
caccatgtacagcctgctggagactgaactcaag

SEQ ID NO: 111 - DNA, 30 bp, synthetic construct
  misc_feature 1..30: note = oligonucleotide
  source 1..30: mol_type = other DNA
tcagatgtgggtcagcggcaccgttccatt

SEQ ID NO: 112 - DNA, 27 bp, synthetic construct
  misc_feature 1..27: note = oligonucleotide
  source 1..27: mol_type = other DNA
cacctcggccatggcctcgctgctggg

SEQ ID NO: 113 - DNA, 27 bp, synthetic construct
  misc_feature 1..27: note = oligonucleotide
  source 1..27: mol_type = other DNA
ctccattcctccagctctatgacacac

SEQ ID NO: 114 - DNA, 28 bp, synthetic construct
  misc_feature 1..28: note = oligonucleotide
  source 1..28: mol_type = other DNA
caccatggcgctgaccagctcctcacaa

SEQ ID NO: 115 - DNA, 24 bp, synthetic construct
  misc_feature 1..24: note = oligonucleotide
  source 1..24: mol_type = other DNA
ttaaaggtgggttactggcatggg

SEQ ID NO: 116 - DNA, 26 bp, synthetic construct
  misc_feature 1..26: note = oligonucleotide
  source 1..26: mol_type = other DNA
caccagagccatgagcagcccggatg

SEQ ID NO: 117 - DNA, 26 bp, synthetic construct
  misc_feature 1..26: note = oligonucleotide
  source 1..26: mol_type = other DNA
cgtcaaatgtcggggtagttgcaata

SEQ ID NO: 118 - DNA, 29 bp, synthetic construct
  misc_feature 1..29: note = oligonucleotide
  source 1..29: mol_type = other DNA
caccatgcagagatcgccgcccggctacg

SEQ ID NO: 119 - DNA, 30 bp, synthetic construct
  misc_feature 1..30: note = oligonucleotide
  source 1..30: mol_type = other DNA
ctagcctgagatgcaagcactgtaatagac

SEQ ID NO: 120 - DNA, 28 bp, synthetic construct
  misc_feature 1..28: note = oligonucleotide
  source 1..28: mol_type = other DNA
caccatgaataatccatcagaaaccaat

SEQ ID NO: 121 - DNA, 24 bp, synthetic construct
  misc_feature 1..24: note = oligonucleotide
  source 1..24: mol_type = other DNA
gctctgcactcagctcactgtgcc

SEQ ID NO: 122 - DNA, 28 bp, synthetic construct
  misc_feature 1..28: note = oligonucleotide
  source 1..28: mol_type = other DNA
caccatggccaccaccgcgcagtatctg

SEQ ID NO: 123 - DNA, 24 bp, synthetic construct
  misc_feature 1..24: note = oligonucleotide
  source 1..24: mol_type = other DNA
ggaacccagtccgcagggtcactg

SEQ ID NO: 124 - DNA, 29 bp, synthetic construct
  misc_feature 1..29: note = oligonucleotide
  source 1..29: mol_type = other DNA
caccatgaggcagaagagagagaggaggc

SEQ ID NO: 125 - DNA, 30 bp, synthetic construct
  misc_feature 1..30: note = oligonucleotide
  source 1..30: mol_type = other DNA
tcagaggtgacgcttcatgtgcagagctaa

SEQ ID NO: 126 - DNA, 30 bp, synthetic construct
  misc_feature 1..30: note = oligonucleotide
  source 1..30: mol_type = other DNA
caccatggcgctcagcgagcctatcttgcc

SEQ ID NO: 127 - DNA, 30 bp, synthetic construct
  misc_feature 1..30: note = oligonucleotide
  source 1..30: mol_type = other DNA
ctacatatgtcgcttcatgtgcaaggccag

SEQ ID NO: 128 - DNA, 28 bp, synthetic construct
  misc_feature 1..28: note = oligonucleotide
  source 1..28: mol_type = other DNA
caccatgcccacgcgggtgctgaccatg

SEQ ID NO: 129 - DNA, 24 bp, synthetic construct
  misc_feature 1..24: note = oligonucleotide
  source 1..24: mol_type = other DNA
tcgctcagttctggtggcgcttca

SEQ ID NO: 130 - DNA, 34 bp, synthetic construct
  misc_feature 1..34: note = oligonucleotide
  source 1..34: mol_type = other DNA
caccatggacttcgactcgtatcagcactatttc

SEQ ID NO: 131 - DNA, 30 bp, synthetic construct
  misc_feature 1..30: note = oligonucleotide
  source 1..30: mol_type = other DNA
ttagtagccactgaggtacgcgattctctt

SEQ ID NO: 132 - DNA, 30 bp, synthetic construct
  misc_feature 1..30: note = oligonucleotide
  source 1..30: mol_type = other DNA
caccatgcccagctgcaccgcgtccaccat

SEQ ID NO: 133 - DNA, 26 bp, synthetic construct
  misc_feature 1..26: note = oligonucleotide
  source 1..26: mol_type = other DNA
ttagcaagtccgagcgtgttcgatct

SEQ ID NO: 134 - protein, 439 aa, synthetic construct
  REGION 1..439: note = peptide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 135 - DNA, 1024 bp, synthetic construct
  misc_feature 1..1024: note = oligonucleotide
  source 1..1024: mol_type = other DNA
atgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagt

SEQ ID NO: 136 - protein, 368 aa, synthetic construct
  REGION 1..368: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMWSGFSARERLERVVSDRLAPGAPRGNPPKAPATPDGTPSLEASNPAPATQCQLGEPKTQACSGSESPSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEDVTKRKNHNFLERKRRNDLRSRFLALRDQVPTLASCSKAPKVVILSKALEYLQALVGAEKKMATEKRQLRCRQQQLQKRIAYLSGY

SEQ ID NO: 137 - DNA, 1024 bp, synthetic construct
  misc_feature 1..1024: note = oligonucleotide
  source 1..1024: mol_type = other DNA
atggacttcgactcgtatcagcactatttctacgactatgactgcggagaggatttctaccgctccacggcgcccagcgaggacatctggaagaaattcgagctggtgccgtcgccccccacgtcgccgccctggggctccggtcccggcgccgtggacccagcctctgggattaatcccggggagccgtggcctggagggggtgccggggacgaggcggaatctcggggccattcgaaagcctggggcaggaattatgcttccatcattcgccgtgactgcatgtggagcggcttctccgcccgagaacggctggagagagtggtgagcgacaggctggccccaggcgcgccccgggggaacccgcccaaagcgcccgctaccccggacggcactcctagtctggaagccagtaacccggcgcccgccacccaatgtcagctgggcgagcccaagactcaggcctgctccgggtccgagagccccagcgattctgaaggtgaagagattgacgtggtgaccgtggagaagaggcgatctctggacatccgaaagccagtcaccatcacggtgcgagcagaccccctggacccctgcatgaagcacttccatatctctatccaccaacagcagcataactatgctgcccgttttcctccagaaagttgctctcaagagggggatcctgagccaggtccccaggaagaggctccggagatagaagctcccaaggagaaagaggaggaggaagaggaagaggaggaagaagagattgtgagccccccacctgtcggaagtgaggctccccagtcctgccaccccaaacctgtcagttctgacactgaggacgtgaccaagaggaagaaccataacttcttggaacgaaaaaggaggaatgacctccgctcccggttcctagccctgcgggaccaggttcccaccctggccagctgctctaaggcccccaaagtcgtgatcctcagcaaggcgttagaatacttgcaggctttggtgggggctgaaa

SEQ ID NO: 138 - protein, 424 aa, synthetic construct
  REGION 1..424: note = peptide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEDVTKRKNHNFLERKRRNDLRSRFLALRDQVPTLASCSKAPKVVILSKALEYLQALVGAEKKMATEKRQLRCRQQQLQKRIAYLSGY

SEQ ID NO: 139 - DNA, 1024 bp, synthetic construct
  misc_feature 1..1024: note = oligonucleotide
  source 1..1024: mol_type = other DNA
atgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctagcgattctgaaggtgaagagattgacgtggtgaccgtggagaagaggcgatctctggacatccgaaagccagtcaccatcacggtgcgagcagaccccctggacccctgcatgaagcacttccatatctctatccaccaacagcagcataactatgctgcccgttttcctccagaaagttgctctcaagagggggatcctgagccaggtccccaggaagaggctccggagatagaagctcccaaggagaaagaggaggaggaagaggaagaggaggaagaagagattgtgagccccccacctgtcggaagtgaggctccccagtcctgccaccccaaacctgtcagttctgacactgaggacgtgacca

SEQ ID NO: 140 - protein, 381 aa, synthetic construct
  REGION 1..381: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMWSGFSARERLERVVSDRLAPGAPRGNPPKAPATPDGTPSLEASNPAPATQCQLGEPKTQACSGSESPSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 141 - DNA, 1024 bp, synthetic construct
  misc_feature 1..1024: note = oligonucleotide
  source 1..1024: mol_type = other DNA
atggacttcgactcgtatcagcactatttctacgactatgactgcggagaggatttctaccgctccacggcgcccagcgaggacatctggaagaaattcgagctggtgccgtcgccccccacgtcgccgccctggggctccggtcccggcgccgtggacccagcctctgggattaatcccggggagccgtggcctggagggggtgccggggacgaggcggaatctcggggccattcgaaagcctggggcaggaattatgcttccatcattcgccgtgactgcatgtggagcggcttctccgcccgagaacggctggagagagtggtgagcgacaggctggccccaggcgcgccccgggggaacccgcccaaagcgcccgctaccccggacggcactcctagtctggaagccagtaacccggcgcccgccacccaatgtcagctgggcgagcccaagactcaggcctgctccgggtccgagagcccctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagcca

SEQ ID NO: 142 - protein, 410 aa, synthetic construct
  REGION 1..410: note = oligonucleotide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLERVVSDRLAPGAPRGNPPKAPATPDGTPSLEASNPAPATQCQLGEPKTQACSGSESPSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 143 - DNA, 1024 bp, synthetic construct
  misc_feature 1..1024: note = oligonucleotide
  source 1..1024: mol_type = other DNA
atgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggagagagtggtgagcgacaggctggccccaggcgcgccccgggggaacccgcccaaagcgcccgctaccccggacggcactcctagtctggaagccagtaacccggcgcccgccacccaatgtcagctgggcgagcccaagactcaggcctgctccgggtccgagagccccagcgattctgaaggtgaagagattgacgtggtgaccgtggagaagaggcgatctctggacatccgaaagccagtcaccatcacggtgcgagcagaccccctggacccctgcatgaagcacttccatatctctatccaccaacagcagcataactatgctgcccgttttcctccagaaagttgctctcaagagggggatcctgagccaggtccccaggaagaggctccggagatagaagctcccaaggagaaagaggaggaggaagaggaagaggaggaagaagagattgtgagccccccacctgtcggaagtgaggctccccagtcctgccaccccaaacctgtctcctcagacacggaggaaaacgacaagaggcggacacacaacgtcttggaacgtcagaggaggaacgagctga

SEQ ID NO: 144 - protein, 397 aa, synthetic construct
  REGION 1..397: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMWSGFSARERLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEDVTKRKNHNFLERKRRNDLRSRFLALRDQVPTLASCSKAPKVVILSKALEYLQALVGAEKKMATEKRQLRCRQQQLQKRIAYLSGY

SEQ ID NO: 145 - DNA, 1024 bp, synthetic construct
  misc_feature 1..1024: note = oligonucleotide
  source 1..1024: mol_type = other DNA
atggacttcgactcgtatcagcactatttctacgactatgactgcggagaggatttctaccgctccacggcgcccagcgaggacatctggaagaaattcgagctggtgccgtcgccccccacgtcgccgccctggggctccggtcccggcgccgtggacccagcctctgggattaatcccggggagccgtggcctggagggggtgccggggacgaggcggaatctcggggccattcgaaagcctggggcaggaattatgcttccatcattcgccgtgactgcatgtggagcggcttctccgcccgagaacggctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggagttctgacactgaggacgtgaccaagaggaagaaccataacttcttggaacgaaaaaggaggaatgacctccgctcccggttcctagccctgcgggaccaggttc

SEQ ID NO: 146 - protein, 437 aa, synthetic construct
  REGION 1..437: note = oligonucleotide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEDVTKRKNHNFLERKRRNDLRSRFLALRDQVPTLASCSKAPKVVILSKALEYLQALVGAEKKMATEKRQLRCRQQQLQKRIAYLSGY

SEQ ID NO: 147 - DNA, 1024 bp, synthetic construct; identical to SEQ ID NO: 135
  misc_feature 1..1024: note = oligonucleotide
  source 1..1024: mol_type = other DNA

SEQ ID NO: 148 - protein, 408 aa, synthetic construct
  REGION 1..408: note = peptide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLERVVSDRLAPGAPRGNPPKAPATPDGTPSLEASNPAPATQCQLGEPKTQACSGSESPSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEDVTKRKNHNFLERKRRNDLRSRFLALRDQVPTLASCSKAPKVVILSKALEYLQALVGAEKKMATEKRQLRCRQQQLQKRIAYLSGY

SEQ ID NO: 149 - DNA, 1024 bp, synthetic construct
  misc_feature 1..1024: note = oligonucleotide
  source 1..1024: mol_type = other DNA
atgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggagagagtggtgagcgacaggctggccccaggcgcgccccgggggaacccgcccaaagcgcccgctaccccggacggcactcctagtctggaagccagtaacccggcgcccgccacccaatgtcagctgggcgagcccaagactcaggcctgctccgggtccgagagccccagcgattctgaaggtgaagagattgacgtggtgaccgtggagaagaggcgatctctggacatccgaaagccagtcaccatcacggtgcgagcagaccccctggacccctgcatgaagcacttccatatctctatccaccaacagcagcataactatgctgcccgttttcctccagaaagttgctctcaagagggggatcctgagccaggtccccaggaagaggctccggagatagaagctcccaaggagaaagaggaggaggaagaggaagaggaggaagaagagattgtgagccccccacctgtcggaagtgaggctccccagtcctgccaccccaaacctgtcagttctgacactgaggacgtgaccaagaggaagaaccataacttcttggaacgaaaaaggaggaatgacctcc

SEQ ID NO: 150 - protein, 370 aa, synthetic construct
  REGION 1..370: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMWSGFSARERLERVVSDRLAPGAPRGNPPKAPATPDGTPSLEASNPAPATQCQLGEPKTQACSGSESPSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 151 - DNA, 1023 bp, synthetic construct
  misc_feature 1..1023: note = oligonucleotide
  source 1..1023: mol_type = other DNA
atggacttcgactcgtatcagcactatttctacgactagactgcggagaggatttctaccgctccacggcgcccagcgaggacatctggaagaaattcgagctggtgccgtcgccccccacgtcgccgccctggggctccggtcccggcgccgtggacccagcctctgggattaatcccggggagccgtggcctggagggggtgccggggacgaggcggaatctcggggccattcgaaagcctggggcaggaattatgcttccatcattcgccgtgactgcatgtggagcggcttctccgcccgagaacggctggagagagtggtgagcgacaggctggccccaggcgcgccccgggggaacccgcccaaagcgcccgctaccccggacggcactcctagtctggaagccagtaacccggcgcccgccacccaatgtcagctgggcgagcccaagactcaggcctgctccgggtccgagagccccagcgattctgaaggtgaagagattgacgtggtgaccgtggagaagaggcgatctctggacatccgaaagccagtcaccatcacggtgcgagcagaccccctggacccctgcatgaagcacttccatatctctatccaccaacagcagcataactatgctgcccgttttcctccagaaagttgctctcaagagggggatcctgagccaggtccccaggaagaggctccggagatagaagctcccaaggagaaagaggaggaggaagaggaagaggaggaagaagagattgtgagccccccacctgtcggaagtgaggctccccagtcctgccaccccaaacctgtctcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagc

SEQ ID NO: 152 - protein, 399 aa, synthetic construct
  REGION 1..399: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMWSGFSARERLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 153 - DNA, 1024 bp, synthetic construct
  misc_feature 1..1024: note = oligonucleotide
  source 1..1024: mol_type = other DNA
atggacttcgactcgtatcagcactatttctacgactatgactgcggagaggatttctaccgctccacggcgcccagcgaggacatctggaagaaattcgagctggtgccgtcgccccccacgtcgccgccctggggctccggtcccggcgccgtggacccagcctctgggattaatcccggggagccgtggcctggagggggtgccggggacgaggcggaatctcggggccattcgaaagcctggggcaggaattatgcttccatcattcgccgtgactgcatgtggagcggcttctccgcccgagaacggctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatcc

SEQ ID NO: 154 - protein, 439 aa, synthetic construct
  REGION 1..439: note = peptide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMESGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 155 - DNA, 1024 bp, synthetic construct
  misc_feature 1..1024: note = oligonucleotide
  source 1..1024: mol_type = other DNA
atgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatggagagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagt

SEQ ID NO: 156 - protein, 439 aa, synthetic construct
  REGION 1..439: note = peptide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVDILKKATAYILSIQADEHKLTSEKDLLRKRREQLKHKLEQLRNSGA

SEQ ID NO: 157 - DNA, 1024 bp, synthetic construct; identical to SEQ ID NO: 135
  misc_feature 1..1024: note = oligonucleotide
  source 1..1024: mol_type = other DNA

SEQ ID NO: 158 - protein, 439 aa, synthetic construct
  REGION 1..439: note = peptide
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLPRKRREQLKHKLEQLRNSGA

SEQ ID NO: 159 - DNA, 1024 bp, synthetic construct; identical to SEQ ID NO: 135
  misc_feature 1..1024: note = oligonucleotide
  source 1..1024: mol_type = other DNA

SEQ ID NO: 160 - protein, 368 aa, synthetic construct
  REGION 1..368: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMESGFSARERLERVVSDRLAPGAPRGNPPKAPATPDGTPSLEASNPAPATQCQLGEPKTQACSGSESPSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEDVTKRKNHNFLERKRRNDLRSRFLALRDQVPTLASCSKAPKVVILSKALEYLQALVGAEKKMATEKRQLRCRQQQLQKRIAYLSGY

SEQ ID NO: 161 - DNA, 1024 bp, synthetic construct
  misc_feature 1..1024: note = oligonucleotide
  source 1..1024: mol_type = other DNA
atggacttcgactcgtatcagcactatttctacgactatgactgcggagaggatttctaccgctccacggcgcccagcgaggacatctggaagaaattcgagctggtgccgtcgccccccacgtcgccgccctggggctccggtcccggcgccgtggacccagcctctgggattaatcccggggagccgtggcctggagggggtgccggggacgaggcggaatctcggggccattcgaaagcctggggcaggaattatgcttccatcattcgccgtgactgcatggagagcggcttctccgcccgagaacggctggagagagtggtgagcgacaggctggccccaggcgcgccccgggggaacccgcccaaagcgcccgctaccccggacggcactcctagtctggaagccagtaacccggcgcccgccacccaatgtcagctgggcgagcccaagactcaggcctgctccgggtccgagagccccagcgattctgaaggtgaagagattgacgtggtgaccgtggagaagaggcgatctctggacatccgaaagccagtcaccatcacggtgcgagcagaccccctggacccctgcatgaagcacttccatatctctatccaccaacagcagcataactatgctgcccgttttcctccagaaagttgctctcaagagggggatcctgagccaggtccccaggaagaggctccggagatagaagctcccaaggagaaagaggaggaggaagaggaagaggaggaagaagagattgtgagccccccacctgtcggaagtgaggctccccagtcctgccaccccaaacctgtcagttctgacactgaggacgtgaccaagaggaagaaccataacttcttggaacgaaaaaggaggaatgacctccgctcccggttcctagccctgcgggaccaggttcccaccctggccagctgctctaaggcccccaaagtcgtgatcctcagcaaggcgttagaatacttgcaggctttggtgggggctgaaa

SEQ ID NO: 162 - protein, 368 aa, synthetic construct
  REGION 1..368: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMWSGFSARERLERVVSDRLAPGAPRGNPPKAPATPDGTPSLEASNPAPATQCQLGEPKTQACSGSESPSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEDVTKRKNHNFLERKRRNDLRSRFLALRDQVPTLASCSKAPKVDILSKALEYLQALVGAEKKMATEKRQLRCRQQQLQKRIAYLSGY

SEQ ID NO: 163 - DNA, 1024 bp, synthetic construct
  misc_feature 1..1024: note = oligonucleotide
  source 1..1024: mol_type = other DNA
atggacttcgactcgtatcagcactatttctacgactatgactgcggagaggatttctaccgctccacggcgcccagcgaggacatctggaagaaattcgagctggtgccgtcgccccccacgtcgccgccctggggctccggtcccggcgccgtggacccagcctctgggattaatcccggggagccgtggcctggagggggtgccggggacgaggcggaatctcggggccattcgaaagcctggggcaggaattatgcttccatcattcgccgtgactgcatgtggagcggcttctccgcccgagaacggctggagagagtggtgagcgacaggctggccccaggcgcgccccgggggaacccgcccaaagcgcccgctaccccggacggcactcctagtctggaagccagtaacccggcgcccgccacccaatgtcagctgggcgagcccaagactcaggcctgctccgggtccgagagccccagcgattctgaaggtgaagagattgacgtggtgaccgtggagaagaggcgatctctggacatccgaaagccagtcaccatcacggtgcgagcagaccccctggacccctgcatgaagcacttccatatctctatccaccaacagcagcataactatgctgcccgttttcctccagaaagttgctctcaagagggggatcctgagccaggtccccaggaagaggctccggagatagaagctcccaaggagaaagaggaggaggaagaggaagaggaggaagaagagattgtgagccccccacctgtcggaagtgaggctccccagtcctgccaccccaaacctgtcagttctgacactgaggacgtgaccaagaggaagaaccataacttcttggaacgaaaaaggaggaatgacctccgctcccggttcctagccctgcgggaccaggttcccaccctggccagctgctctaaggcccccaaagtcgacatcctcagcaaggcgttagaatacttgcaggctttggtgggggctgaaa

SEQ ID NO: 164 - protein, 365 aa, synthetic construct
  REGION 1..365: note = peptide
MDFDSYQHYFYDYDCGEDFYRSTAPSEDIWKKFELVPSPPTSPPWGSGPGAVDPASGINPGEPWPGGGAGDEAESRGHSKAWGRNYASIIRRDCMWSGFSARERLERVVSDRLAPGAPRGNPPKAPATPDGTPSLEASNPAPATQCQLGEPKTQACSGSESPSDSEGEEIDVVTVEKRRSLDIRKPVTITVRADPLDPCMKHFHISIHQQQHNYAARFPPESCSQEGDPEPGPQEEAPEIEAPKEKEEEEEEEEEEEIVSPPPVGSEAPQSCHPKPVSSDTEDVTKRKFLERKRRNDLRSRFLALRDQVPTLASCSKAPKVVILSKALEYLQALVGAEKKMATEKRQPRCRQQQLQKRIAYLSGY

SEQ ID NO: 165 - DNA, 1024 bp, synthetic construct; identical to SEQ ID NO: 137
  misc_feature 1..1024: note = oligonucleotide
  source 1..1024: mol_type = other DNA

(19) United States
(12) Patent Application Publication
(10) Pub. No.: US 2026/0317385 A1
(43) Pub. Date: Oct. 15, 2026

(54) NUCLEAR REPROGRAMMING FACTOR AND INDUCED PLURIPOTENT STEM CELLS

(71) Applicant: Kyoto University, Kyoto-shi, Kyoto (JP)

(72) Inventors: Shinya Yamanaka, Kyoto (JP); Kazutoshi Takahashi, Kyoto (JP); Masato Nakagawa, Kyoto (JP)

(21) Appl. No.: 19/360,776

(22) Filed: Oct. 16, 2025

Related U.S. Application Data
(63) Continuation of application No. 17/445,282, filed on Aug. 17, 2021, now Pat. No. 12,466,863.
Application No. 17/445,282 is a continuation of application No. 15/474,855, filed on Mar. 30, 2017, now abandoned.
Application No. 15/474,855 is a continuation of application No. 12/289,873, filed on Nov. 6, 2008, now abandoned.
Application No. 12/289,873 is a continuation-in-part of application No. 12/213,035, filed on Jun. 13, 2008, now Pat. No. 8,278,104.
Application No. 12/213,035 is a continuation-in-part of application No. PCT/JP2006/324881, filed on Dec. 6, 2006.
(60) Provisional application No. 61/001,108, filed on Oct. 31, 2007.
(60) Provisional application No. 60/996,289, filed on Nov. 9, 2007.

(30) Foreign Application Priority Data
Dec. 13, 2005 (JP) 2005-359537

(51) Int. Cl. C07K 14/47; C12N 5/074

(52) U.S. Cl. CPC C07K 14/4702; C12N 5/0696; C12N 2501/60; C12N 2501/602; C12N 2501/603; C12N 2501/604; C12N 2501/606; C12N 2510/00; C12N 2740/15043

(57) ABSTRACT

A method for preparing an induced pluripotent stem cell from a somatic cell, comprising introducing a nuclear reprogramming factor into a somatic cell, wherein the method includes a step of selecting a nuclear reprogramming factor comprising a plurality of genes selected from the following 24 candidates of genes relevant to nuclear reprogramming: ECAT1, ECAT2, ECAT3, ECAT4, ECAT5, ECAT7, ECAT8, ECAT9, ECAT10, ECAT15-1, ECAT15-2, Fthl17, SaI14, Oct3/4, Sox2, Rex1, Utf1, Tel1, Stella, KLf4, ß-catenin, c-Myc, Stat3, and Grb2; and wherein by using the nuclear reprogramming factor, the nucleus of a somatic cell can be reprogrammed to obtain an induced pluripotent stem cell.

### PRIOR RELATED APPLICATIONS

This application is a continuation application of U.S. application Ser. No. 17/445,282, filed Aug. 17, 2021, which is a continuation application of U.S. application Ser. No. 15/474,855, filed Mar. 30, 2017, which is a continuation application of U.S. application Ser. No. 12/289,873, filed Nov. 6, 2008, which is a continuation-in-part of U.S. application Ser. No. 12/213,035, filed Jun. 13, 2008, which is a continuation-in-part of PCT/JP2006/324881, filed Dec. 6, 2006, which claims priority to Japanese Application No. 2005-359537, filed Dec. 13, 2005, and also claims priority to U.S. Provisional Application No. 61/001,108, filed Oct. 31, 2007, and U.S. Provisional Application No. 60/996,289, filed Nov. 9, 2007. The entire disclosures of each of the above-cited applications are considered as being part of this application and are expressly incorporated by reference herein in their entireties.

### FIELD OF THE INVENTION

The present invention relates to a nuclear reprogramming factor having an action of reprogramming a somatic cell to derive an induced pluripotent stem (iPS) cell. The present invention also relates to the aforementioned iPS cells, methods of generating. and maintaining iPS cells, and methods of using iPS cells, including screening and testing methods as well as methods of stem cell therapy. The present invention also relates to somatic cells derived by inducing differentiation of the aforementioned iPS cells.

### REFERENCE TO SEQUENCE LISTING

A Sequence Listing submitted as an XML file via Patent Center is hereby incorporated by reference in accordance with 35 U.S.C. § 1.52(e). The name of the XML file for the Sequence Listing is 62231060_1.xml, the date of creation of the XML file is Oct. 10, 2025, and the size of the XML file is 234,944 bytes.

### BACKGROUND OF THE INVENTION

Embryonic stem cells (ES cells) are stem cells established from human or mouse early embryos which have a characteristic feature that they can be cultured over a long period of time while maintaining pluripotent ability to differentiate into all kinds of cells existing in living bodies. Human embryonic stem cells are expected for use as resources for cell transplantation therapies for various diseases such as Parkinson's disease, juvenile diabetes, and leukemia, taking advantage of the aforementioned properties. However, transplantation of ES cells has a problem of causing rejection in the same manner as organ transplantation. Moreover, from an ethical viewpoint, there are many dissenting opinions against the use of ES cells which are established by destroying human embryos.

Embryonic stem (ES) cells, derived from the inner cell mass of mammalian blastocysts, have the ability to grow indefinitely while maintaining pluripotency (Evans et al. Nature 292:154-156, 1981; Martin P.NA.S. USA 78:7634-7638, 1981). These properties have led to expectations that human ES cells might be useful to understand disease mechanisms, to screen effective and safe drugs, and to treat patients of various diseases and injuries, such as juvenile diabetes and spinal cord injury (Thomson et al. Science 282:1145-114?, 1998). Use of human embryos, however, faces ethical controversies that hinder the applications of human ES cells. In addition, it is difficult to generate patient- or disease-specific ES cells, which are required for their effective application. Therefore, if dedifferentiation of a patient's own somatic cells could be induced to establish cells having pluripotency and growth ability similar to those of ES cells (in this specification, these cells are referred to as “induced pluripotent stem cells (iPS cells)”, though they are sometimes called “embryonic stem cell-like cells” or “ES-like cells”), it is anticipated that such cells could be used as ideal pluripotent cells, free from rejection or ethical difficulties.

Methods for nuclear reprogramming of a somatic cell nucleus have been reported. One technique for nuclear reprogramming which has been reported involves nuclear transfer into oocytes (Wakayama et al. Nature 394:369-374, 1998; Wilmut et al. Nature 385:810-813, 1997). Another method, for example, a technique of establishing an embryonic stem cell from a cloned embryo, prepared by transplanting a nucleus of a somatic cell into an egg, was reported (Hwang et al. Science 303:1669-74, 2004; Hwang et al. Science 308:1777-83, 2005): these articles were, however, proved to be fabrications and later withdrawn. Others have reported techniques for nuclear reprogramming of a somatic cell nucleus by fusing a somatic cell and an ES cell (Tada et al. Curr. Biol. 11:1553-1558, 2001; Cowan et al. Science 309:1369-73, 2005). Another reported technique for reprogramming a cell nucleus involves treatment of a differentiated cell with an undifferentiated human carcinoma cell extract (Taranger et al. Mol. Biol. Cell 16:5719-35, 2005). However, these methods all have serious drawbacks. Methods of nuclear transfer into oocytes and techniques which involve the fusion of ES and differentiated cells both comprise the use of ES cells, which present ethical problems. In addition, cells generated by such methods often lead to problems with rejection upon transplantation into an unmatched host. Furthermore, the use of cell extracts to treat differentiated cells is technically unreliable and unsafe, in part because the cell extract components responsible for the nuclear programming are mixed in solution with other unknown factors.

A method for screening a nuclear reprogramming factor having an action of reprogramming differentiated somatic cells to derive induced pluripotent stems cell was proposed in International Publication WO2005/80598, which is incorporated by reference in its entirety. This method comprises the steps of: contacting somatic cells containing a marker gene under expression regulatory control of an ECAT (ES cell associated transcript) gene expression control region with a test substance; examining presence or absence of the appearance of a cell that expresses the marker gene; and choosing a test substance inducing the appearance of said cell as a candidate nuclear reprogramming factor for somatic cells. A method for reprogramming a somatic cell is disclosed in Example 6 and the like of the above publication. However, this publication fails to report an actual identification of a nuclear reprogramming factor.

In view of these problems, there remains a need in the art for nuclear reprogramming factors capable of generating pluripotent stem cells from somatic cells. There also remains a need for pluripotent stem cells, which can be derived from a patient's own somatic cells, so as to render ethical issues and avoid problems with rejection. Such cells would have enormous potential for both research and clinical applications.

### SUMMARY OF THE INVENTION

The present invention provides induced pluripotent stem (iPS) cells derived by nuclear reprogramming of a somatic cell. The present invention also provides methods for reprogramming of a differentiated cell without using eggs, embryos, or embryonic stem (ES) cells. The present invention also provides nuclear reprogramming factors for induction of pluripotent stem cells. The disclosed methods and nuclear reprogramming factors may be used to conveniently and highly reproducibly establish iPS cells having pluripotency and growth ability similar to that of ES cells. More specifically, the present invention provides for inducing reprogramming of a differentiated cell without using eggs, embryos, or ES cells to conveniently and highly reproducibly establish the iPS cells having pluripotency and growth ability similar to that of ES cells.

The invention provides a pluripotent stem cell induced by reprogramming a somatic cell in the absence of eggs, embryos, or embryonic stem (ES) cells. The somatic cell can be a mammalian cell, for example a mouse cell or a human cell. The present invention also provides such a pluripotent stem cell, wherein the reprogramming comprises contacting the somatic cell with a nuclear reprogramming factor.

The nuclear reprogramming factor can comprise at least one gene product, for example a protein. The nuclear reprogramming factor can comprise a gene product of an Oct family gene, a Klf family gene, a Myc family gene, or a Sox family gene. The nuclear reprogramming factor can comprise one or more gene products of each of: an Oct family gene, a Klf family gene, and a Sox family gene. The nuclear reprogramming factor can comprise one or more gene products of each of: an Oct family gene, a Klf family gene, a Myc family gene, and a Sox family gene. Furthermore, the nuclear reprogramming factor can comprise one or more gene products of each of: an Oct family gene, a Klf family gene, together with a cytokine. The cytokine can be at least one of basic fibroblast growth factor (bFGF) and stem cell factor (SCF).

The invention also provides a method for preparing an induced pluripotent stem cell by nuclear reprogramming of a somatic cell, which comprises contacting a nuclear reprogramming factor with the somatic cell to obtain an induced pluripotent stem cell. The invention also provides such a method which is performed in the absence of eggs, embryos, or embryonic stem (ES) cells. The present invention also provides an induced pluripotent stem cell obtained by such a method. The present invention also provides a pluripotent stem cell induced by reprogramming a somatic cell, wherein the reprogramming comprises contacting the somatic cell with a nuclear reprogramming factor.

The present invention also provides such a method wherein the nuclear reprogramming factor comprises one or more gene products of each of: an Oct family gene, a Klf family gene, and a Sox family gene. The present invention also provides such a method wherein the nuclear reprogramming factor comprises one or more gene products of each of: Oct3/4, Klf4, and Sox2. The present invention also provides such a method wherein the nuclear reprogramming factor further comprises one or more gene products of a Sall4 gene. The present invention also provides pluripotent stem cells prepared by such methods.

The present invention also provides such a method wherein the nuclear reprogramming factor comprises one or more gene products of each of: wherein the nuclear reprogramming factor comprises one or more gene products of each of: an Oct family gene, a Klf family gene, a Myc family gene, and a Sox family gene. The present invention also provides such a method wherein the nuclear reprogramming factor comprises one or more gene products of each of: Oct3/4, Klf4, c-Myc, and Sox2. The present invention also provides such a method wherein the nuclear reprogramming factor further comprises one or more gene products of a Sall4 gene. The present invention also provides pluripotent stem cells prepared by such methods.

The present invention also provides such a method wherein the nuclear reprogramming factor comprises one or more gene products of each of: Klf4, c-Myc, Oct3/4, Sox2, Nanog, and Lin28. The present invention also provides pluripotent stem cells prepared by such a method.

The present invention also provides a method of inducing a somatic cell to become a pluripotent stem cell comprising contacting the somatic cell with a nuclear reprogramming factor under conditions to obtain a pluripotent stem cell free of rejection.

The present invention also provides a somatic cell derived by inducing differentiation of an induced pluripotent stem cell as disclosed herein.

The present invention also provides a pluripotent stem cell induced by reprogramming a somatic cell, wherein the reprogramming is performed in the absence of eggs, embryos, or embryonic stem (ES) cells and comprises contacting the somatic cell with a nuclear reprogramming factor comprising one or more gene products of a Sall1 gene and/or a Sall4 gene.

The present invention also provides a pluripotent stem cell induced by reprogramming a somatic cell, wherein the reprogramming is performed in the absence of eggs, embryos, or embryonic stem (ES) cells and comprises contacting the somatic cell with a nuclear reprogramming factor comprising one or more gene products of: an L-Myc gene, an N-Myc gene, and/or a c-Myc gene.

The present invention also provides a pluripotent stem cell induced by reprogramming a somatic cell, wherein the reprogramming is performed in the absence of eggs, embryos, or embryonic stem (ES) cells and comprises contacting the somatic cell with a nuclear reprogramming factor comprising one or more gene products of a Lin28 gene.

The present invention also provides a method for preparing an induced pluripotent stem cell by nuclear reprogramming of a somatic cell, which comprises contacting a nuclear reprogramming factor comprising one or more gene products of a Sall1 gene and/or a Sall4 gene with the somatic cell to obtain an induced pluipotent stem cell.

The present invention also provides a method for preparing an induced pluripotent stem cell by nuclear reprogramming of a somatic cell, which comprises contacting a nuclear reprogramming factor comprising one or more gene products of: an L-Myc gene, an N-Myc gene, and/or a c-Myc gene with the somatic cell to obtain an induced pluipotent stem cell.

The present invention is also directed to a method for preparing an induced pluripotent stem cell by nuclear reprogramming of a somatic cell, which comprises contacting a nuclear reprogramming factor comprising one or more gene products of a Lin28 gene with the somatic cell to obtain an induced pluipotent stem cell.

The present invention also provides induced pluripotent stem cells obtained by methods disclosed herein.

The somatic cell can be a human cell or a mouse cell.

The nuclear reprogramming factor can comprise one or more gene products of a human or mouse Sall4 gene.

The nuclear reprogramming factor can comprise one or more gene products of each of: an Oct family gene, a Klf family gene, and a Sox family gene.

The nuclear reprogramming factor can comprise one or more gene products of a Myc family gene.

The nuclear reprogramming factor can comprise Oct3/4, Klf4, and Sox2, and at least one of Sall1 and Sall4.

The nuclear reprogramming factor can comprise Oct3/4, Klf4, Sox2, and c-Myc, and at least one of Sall1 and Sall4.

The nuclear reprogramming factor can comprise one or more gene products of each of: an Oct family gene, a Sox family gene, and a Nanog family gene.

The nuclear reprogramming factor can comprise one or more gene products of a Lin28 family gene.

The nuclear reprogramming factor can comprise Oct3/4, Sox2, and Nanog, and at least one of Sall1 and Sall4.

The nuclear reprogramming factor can comprise Oct3/4, Sox2, Nanog, and Lin28, and at least one of Sall1 and Sall4.

The L-Myc gene, the N-Myc gene, and/or the c-Myc gene can be a chimera or mutant gene.

The nuclear reprogramming factor can comprise Ms-cL-Myc or its human homolog.

The nuclear reprogramming factor can comprise amino acids 1-144 of c-Myc or its human homolog.

The nuclear reprogramming factor can comprise amino acids 105-277 of L-Myc or its human homolog.

The nuclear reprogramming factor can comprise amino acids 347-439 of c-Myc, amino acids 278-368 of L-Myc, or their human homolog.

The nuclear reprogramming factor can comprise one or more gene products of: an Oct family gene, and a Klf family gene.

The nuclear reprogramming factor can comprise one or more gene products of a Sox family gene.

The nuclear reprogramming factor can comprise one or more gene products of a Lin28 family gene.

The nuclear reprogramming factor can comprise Oct3/4, Klf4, and L-Myc.

The nuclear reprogramming factor can comprise Oct3/4, Klf4, Sox2, and L-Myc.

The nuclear reprogramming factor can comprise Oct3/4, Klf4, Sox2, L-Myc, and Lin28.

The present invention also provides a method for stem cell therapy comprising: (I) isolating and collecting a somatic cell from a patient; (2) inducing said somatic cell from the patient into an iPS cell (3) inducing differentiation of said iPS cell, and (4) transplanting the differentiated cell from (3) into the patient.

The present invention also provides a method for evaluating a physiological function of a compound comprising treating cells obtained by inducing differentiation of an induced pluripotent stem cell as disclosed herein with the compound.

The present invention also provides a method for evaluating the toxicity of a compound comprising treating cells obtained by inducing differentiation of an induced pluripotent stem cell as disclosed herein with the compound.

Other features and advantages of the present invention will be set forth in the description of the invention that follows, and will be apparent, in part, from the description or may be learned by practice of the invention. The invention will be realized and attained by the compositions, products, and methods particularly pointed out in the written description and claims hereof.

### BRIEF DESCRIPTION OF THE DRAWINGS

FIG. 1 shows a screening method for reprogramming factors using embryonic fibroblasts (MEFs) of a mouse having βgeo knock-in Fbx15 gene.

FIG. 2 depicts photographs showing morphology of iPS cells obtained by introducing the 24 genes shown in TABLE 4. Morphologies of differentiated cells (MEF) and of normal embryonic stem cells (ES) are also shown as a reference.

FIG. 3 shows expression profiles of marker genes in iPS cells. The results of RT-PCR using total RNAs extracted from iPS cells, ES cells and MEF cells as templates are shown.

FIG. 4 shows methylation status of DNA in iPS cells. Genomic DNAs extracted from iPS cells, ES cells, and MEF cells were treated with bisulfite. The target DNAs were amplified by PCR and then inserted into plasmid. Ten clones of plasmid were isolated for each of the genes, and sequenced. Methylated CpGs are indicated with closed circles, and unmethylated CpGs with open circles.

FIG. 5 shows colony numbers of G418-resistant cells obtained by transduction of 24-gene group and 23-gene groups wherein each individual gene was withdrawn from the 24-gene group. The lower parts of the graph show colony numbers obtained in one week after the G418 selection, and the upper parts of the graph show numbers of clones obtained in three weeks. When each boxed gene (the reference number for each gene is the same as that indicated in TABLE 4) was withdrawn, no colonies were obtained at all, or only a few colonies were observed after 3 weeks.

FIG. 6 shows colony numbers of G418-resistant cells obtained by transduction 1 of 10-gene group and 9-gene groups wherein each individual gene was withdrawn from the 10-gene group. When each of genes #14, #15 or #20 was withdrawn, no colony was obtained. When gene #22 was withdrawn, a few G418-resistant colonies were obtained. However, the cells gave differentiated morphology which was apparently different from that of iPS cells.

FIG. 7 shows numbers of G418-resistant emerged colonies (reprogrammed colony) with 10-gene group, 4-gene group, 3-gene groups, or 2-gene groups. Typical morphology and sizes of the colonies are shown.

FIG. 8 depicts photographs showing results of hematoxylin-eosin (H & E) staining of tumors formed after subcutaneous transplantation of iPS cells derived from MEFs into nude mice. Differentiation into a variety of tissues in a triploblastic system was observed.

FIG. 9 depicts photographs of embryos prepared by transplanting iPS cells derived from adult dermal fibroblasts into mouse blastocysts and transplanting the cells into the uteri of pseudo-pregnant mice. It can be observed that, in the upper left embryo, cells derived from the iPS cells (emitting green fluorescence) were systemically distributed. In the lower photographs, it can be observed that almost all cells of the heart, liver, and spinal cord of the embryo were GFP-positive and were derived from the iPS cells.

FIG. 10 depicts photographs showing results of RT-PCR confirming the expression of the ES cell marker genes. In the photographs, Sox2 minus indicates iPS cells established by the transduction of 3 genes into MEFs, 4ECATs indicates iPS cells established by the transduction of 4 genes into MEFs, 10ECATs indicates iPS cells established by the transduction of 10 genes into MEFs, 10ECATs Skin fibroblast indicates iPS cells established by the transduction of 10 genes into dermal fibroblasts, ES indicates mouse ES cells, and MEF indicates MEF cells without gene transduction. The numerical values under the symbols indicate clones numbers.

FIG. 11 shows an effect of bFGF on the establishment of iPS cells from MEFs. Four factors (upper row) or three factors except for c-Myc (lower row) were retrovirally transduced into MEFs derived from Fbx15^{βgeo/βgeo} mice, and cultured on ordinary feeder cells (STO cells) (left) and bFGF expression vector-introduced STO cells (right). G418 selection was performed for 2 weeks, and cells were stained with crystal blue and photographed. The numerical values indicate the number of colonies.

FIG. 12 depicts explanations of the experiments using Nanog-EGFP-IRES-Puro^{r} mice. (A) E. coli artificial chromosome (BAC) containing the mouse Nanog gene in the center was isolated, and the EGFP-IRES-Puro^{r} cassette was inserted upstream from the coding region of Nanog by recombineering. (B) Transgenic mice were prepared with the modified BAC. GFP expression was observed limitedly in inner cell masses of blastocysts and gonads.

FIG. 13 depicts explanations of the experiments using Nanog-EGFP-IRES-Puro^{r} mice. From embryos of Nanog-EGFP-IRES-Puro^{r} mice (13.5 days after fertilization), heads, viscera and gonads were removed to establish MEFs. As a result of analysis with a cell sorter, almost no GFP-positive cells existed in MEFs derived from the Nanog-EGFP-IRES-Puro mice (Nanog) in the same manner as the Fbx15^{βgeo/βgeo} mouse-derived MEFs (Fbx 5) or wild-type mouse-derived MEFs (Wild).

FIG. 14 depicts photographs of iPS cells established from the Nanog-EGFP-IRES-Puro mouse MEFs (left) and the Fbx15^{βgeo/βgeo} mouse MEFs (right). The cells were selected with puromycin and G418, respectively.

FIG. 15 shows results of growth of iPS cells. 100,000 cells of each of ES cells, iPS cells derived from the Nanog-EGFP-IRES-Puro mouse MEFs (Nanog iPS, left), and iPS cells derived from the Fbx15^{βgeo/βgeo} mouse MEFs (Fbx iPS) were seeded on 24-well plates, and passaged every 3 days. Cell count results are shown. The numerical values represent average doubling times.

FIG. 16 shows gene expression profiles of iPS cells. Expression of the marker genes in MEFs, ES cells, iPS cells derived from Nanog-EGFP-IRES-Puro mouse MEFs (Nanog iPS, left), and iPS cells derived from Fbx15^{βgeo/βgeo} mouse MEFs (Fbx iPS) Were analyzed by RT-PCR. The numerical values at the bottom indicate the numbers of passages.

FIG. 17 shows teratoma formation from the Nanog iPS cells. 1,000,000 cells of each of ES cells or Nanog iPS cells #24 (passage 8 times) were subcutaneously injected into the backs of nude mice, and the appearance of tumors formed after 3 weeks (left) and tissue images (right, H & E stained) are shown.

FIG. 18 shows preparation of chimeric mice with the Nanog iPS cells. The chimeric mice that were born after transplantation of the Nanog iPS cells (clone NPMF4EK-24, passaged 6 times) into the blastocysts. Four chimeric mice were born from 90 transplanted embryos.

FIG. 19 shows germ-line transmission from the Nanog iPS cells. PCR analysis of genomic DNA of mice, born by mating of the chimeric mice shown in FIGS. 18 and C57BL/6 mice, revealed the existence of transgenes of Oct3/4 and Klf4 in all of the mice, thereby confirming germ-line transmission.

FIG. 20 shows induction of differentiation into nerve cells from iPS cells. Nerve cells (top, βIII tubulin-positive), oligodendrocytes (left, O4-positive), and astrocytes (right, GFAP-positive) differentiated in vitro from dermal fibroblasts-derived iPS cells are shown.

FIG. 21 depicts explanations of establishment of the iPS cells without using drug selection. MEFs at 10,000 to 100,000 cells per 10 cm dish were seeded, and the 4 factors were retrovirally transduced. No colony appeared in the control (Mock, top left), whilst in the dish with the transduction by the 4 factors, swelling colonies similar to those of the iPS cells were obtained (bottom left and center), as well as flat transformant colonies. When the cells were passaged, cells similar to the iPS cells were obtained (right).

FIG. 22 shows gene expression profiles of cells established without using drug selection. RNA was extracted from the established cells shown in FIG. 21, and expression of the ES cell marker genes was analyzed by RT-PCR.

FIG. 23 shows iPS cell-like cells derived from human fibroblasts. The colonies obtained by retroviral transduction with human homologous genes of the 4 factors into fibroblasts derived from human embryos (A), and the cells after two passages (B) are shown.

FIG. 24 shows establishment of the iPS cells from human adult dermal fibroblasts. The factors mentioned in the left column were transduced retrovirally into human adult dermal fibroblasts infected with the mouse retroviral receptor with lentivirus. The photographs shows phase contrast images (object x 10) on day 8 after the viral infection.

FIG. 25 shows the results of alkaline phosphatase staining of iPS cells from two different experiments. (A) 8×10⁵ HDFs derived from adult skin expressing mouse Slc7a1 gene and introduced with pMXs encoding the genes indicated were plated on mitomycin C-treated STO cells. The infected cells were cultured in ES medium for 12 days. The cells were stained with alkaline phosphatase. (B) BJ fibroblasts expressing mouse Slc7a1 gene were plated at 8×10⁵ cells per 100 mm dish on mitomycin C treated STO cells. Next day, the cells were transduced with the genes indicated (left) by retroviral infection. After transduction, the cells were maintained in ES medium for 2 weeks. After picking up the colonies, the cells were stained with alkaline phosphatase.

FIG. 26 shows Cyanine 3(Cy-3) staining of iPS (-like) cells with ES cell markers. (A) iPS (-like) cells derived from adult human dermal fibroblasts (HDFs) were plated at 5×10⁴ cells per well of 6 well plates on mitomycin C-treated STO cells, and grown for 4 days. The cells were fixed with PBS containing 10% formalin, and blocked with blocking buffer (0.1% bovine serum albumin and 10 mg/ml normal donkey serum in PBS) for 45 minutes at room temperature. Primary antibodies indicated above were diluted 1:100 in blocking buffer. Overnight incubation with primary antibody, the cells were washed with PBS, and then incubated with secondary antibody. Cy-3-conjugated anti-mouse IgG (for ABCG-2 and SSEA-4) and anti-rat IgM (for SSEA-3) antibodies were used. (B) iPS (-like) cells derived from adult HDFs (clone 87E3, 87E4 and 87E12) were plated at 5×10⁴ cells per well of a 6 well plate on mitomycin C-treated STO cells, and grown for 5 days. Parental HDFs also plated on 6 well plate and maintained for 2 days. The cells were fixed with PBS containing 10% formalin, and blocked with blocking buffer (3% BSA in PBS) for 45 minutes at room temperature. Primary antibodies indicated above were diluted 1:100 in blocking buffer. Overnight incubation with primary antibody, the cells were washed with PBS, and then incubated with secondary antibody. Cy-3-conjugated anti-mouse lgG (for ABCG-2, E-cadherin, and SSEA-4) and anti-rat IgM (for SSEA-3) antibodies were used as secondary antibodies.

FIG. 27 shows human iPS (-like) cells express ECATs. Total RNA was isolated from human iPS (-like) cells (iPS-HDFaSlc-87E-1~8, 11 and 12), NTERA2 cloneD1 human embryonic carcinoma cells (passage 35) and adult HDFs expressing mouse Slc7a1 gene (passage 6). First-strand cDNA was synthesized by using oligo-dT20 primer and Rever Tra Ace-α-kit (Toyobo) according to manufacturer's protocol. PCR was performed with the primers as follows: hOct4 S1165 and hOct4-AS1283 for endogenous OCT4, hSox2-S1430 and hSox2-AS1555 for endogenous SOX2, ECAT4-macaca-968S and ECAT4-macaca-1334AS for NANOG, hREX1-RT-U and hREX1-RT-L for REX1, hFGF4-RT-U and hFGF4-RT-L for FGF4, hGDF3-S243 and hGDF3-AS850 for GDF3, hECAT15-S532 and hECAT15-AS916 for ECAT15-1, hECAT15-2-S85 and hECAT15-2-AS667 for ECAT15-2, hpH34-S40 and hpH34-AS259 for ESG1, hTERT-S3556 and hTERT-AS3713 for hTERT, and G3PDH-F and G3PDH-R for G3PDH.

FIG. 28 shows human iPS (-like) cells express ECATs. Total RNA was isolated from human iPS (-like) cells (iPS-BJSlc-97E-1, 2, 4, 5, 6, 7, 8, 10, 11, 12, -97G-3,5, -97H-3, 5), NTERA2 clone D1 human embryonic carcinoma cells (passage 35) and BJ fibroblasts expressing mouse Slc7a1 gene (passage 6). First-strand cDNA was synthesized by using oligo-dT20 primer and Rever Tra Ace-α-kit (Toyobo) according to manufacturer's protocol. PCR was performed with the primers as follows: hOct4 S1165 and hOct4-AS 1283 for endogenous OCT4, hSox2-S1430 and hSox2-AS1555 for endogenous SOX2, ECAT4-macaca-968S and ECAT4-macaca-1334AS for NANOG, hREX1-RT-U and hREX1-RT-L for REX1, hFGF4-RT-U and hFGF4-RT-L for FGF4, hGDF3-S243 and hGDF3-AS850 for GDF3, hECAT15-S532 and hECAT15-AS916 for ECAT15-1, hECAT15-2-S85 and hECAT15-2-AS667 for ECAT15-2, hpH34-S40 and hpH34-AS259 for ESG1, hTERT-S3556 and hTERT-AS3713 for hTERT, and G3PDH-F and G3PDH-R for G3PDH.

FIG. 29 shows teratoma formation. Five million of hiPS (-like) cells were subcutaneously injected into dorsal flanks of SCID mouse (female, 5 weeks old). Two months after injection, large tumors were observed. Tumors were dissected, weighed and photographed. Then these tumors were fixed with PBS containing I 0% formalin. Paraffin-embedded tumor was sliced and then stained with hematoxylin and eosin. (A) Mouse from clone iPS-HDFa/Slc-87E-12. (B)-(D) indicate mouse teratomas from clones iPS-HDFa/Slc-97E-3 (B); iPS-HDFa/Slc-87E-6 (C); and iPS-HDFa/Slc-87E-12 (D).

FIG. 30 shows in vitro differentiation of human iPS-like cells. The cells (iPS-HDFaSlc-127F2, E3) were suspended in hES medium (w/o bFGF). 2×10⁶ cells were transferred to HEMA (2-hydroxyethyl methacrylate)-coated 100 mm tissue culture dish. The medium was changed every other day. After seven days floating culture, the cells were collected, plated to six gelatinized 35 mm dishes and incubated another 7 days. The cells were fixed with PBS containing 10% formalin for 10 min at room temperature, permeabilized with PBS containing 0.5% TritonX-100 for 5 min at room temperature, and blocked with PBS containing 3% BSA for 30 min at room temperature. Primary antibodies used in this experiment were as follows; anti-a-smooth muscle actin (Ms mono, pre-diluted, DAKO), anti-βIII-tubulin (Ms mono, 1:100 in blocking buffer, Chemicon), anti-α-fetoprotein (Rb poly, pre-diluted, DAKO), normal mouse IgG (2 mg/ml, Chemicon), and normal rabbit IgG (2 mg/ml, Chemicon). After incubation with primary antibody (1 hour at room temperature), the cells were washed with PBS, and incubated with secondary antibody (1:300 in blocking buffer).

FIG. 31 shows improved transduction efficiency of retroviruses in human HDFs. HDFs or HDFs expressing mouse Slc7a1 gene (HDF-Slc7a1) were introduced with ecotropic (Eco) or amphotropic (Ampho) pMX retroviruses containing the GFP cDNA. Shown are results of fluorescent microscope (upper) and flow cytometry (lower). Bars=100 μm.

FIG. 32 shows induction of iPS cells from adult HDFs in primate ES cell media. (A) Time schedule of iPS cell generation. (B) Morphology of HDFs. (C) Typical image of non-ES cell-like colony. (D) Typical image of hES cell-like colony. (E) Morphology of established iPS cell line at passage number 6 (clone 20187). (F) Image of iPS cells with high magnification. (G) Spontaneously differentiated cells in the center part of human iPS cell colonies. (H-N) Immunocytochemistry for SSEA-1 (H), SSEA-3 (I), SSEA-4 (J), TRA-1-60 (K), TRA-1-81 (L), TRA-2-4916E (M), and Nanog (N). Nuclei were stained with Hoechst 33342 (blue). Bars=200 μm (B-E, G), 20 μm (F), and 100 μm (H-N).

FIG. 33 shows feeder dependency of human iPS cells. (A) Image of iPS cells plated on gelatin-coated plate. (B) Images of iPS cells cultured on Matrigel-coated plates in MEF-conditioned medium (MEF-CCM). (C) Images of iPS cells cultured in ES medium on Matrigel-coated plates with non-conditioned hES medium.

FIG. 34 shows expression of hES cell marker genes in human iPS cells. (A) RT-PCR analysis of ES cell marker genes. (B) Western blot analysis of ES cell marker genes. (C) Quantitative PCR for expression of retroviral transgenes. The graph shows the average of three assays. Bars indicate standard deviation. (D) Bisulfite genomic sequencing of the promoter regions of OCT3/4, REX1 and NANOG. Open and closed circles indicate unmethylated and methylated CpGs. (E) Luciferase assays. The graphs show the average of the results from four assays. Bars indicate standard deviation.

FIG. 35 shows high levels of telomerase activity and exponential proliferation of human iPS cells. (A) Detection of telomerase activities by the TRAP method. Heat-inactivated (+) samples were used as negative controls. IC=internal control. (B) Growth curve of iPS cells. Shown are averages and standard deviations in quadruplicate.

FIG. 36 shows genetic analyses of human iPS cells. (A) Genomic PCR revealed integration of all the four retroviruses in all clones. (B) Southern blot analyses with a c-MYC cDNA probe. Asterisk indicates the endogenous c-MYC alleles (2.7 kb). Arrowhead indicates mouse c-Myc alleles derived from SNL feeder cells (9.8 kb).

FIG. 37 shows embryoid body-mediated differentiation of human iPS cells. (A) Floating culture of iPS cells at day 8. (B-E) Images of differentiated cells at day 16 (B), neuron-like cells (C), epithelial cells (D), and cobblestone-like cells (E). (F-K) Immunocytochemistry of alpha-fetoprotein (F), vimentin (G), a-smooth muscle actin (H), desmin (I), βIII-tubulin (J), and OFAP (K). Bars=200 μm (A, B) and 100 μm (C-K). Nuclei were stained with Hoechst 33342 (blue). (L) RT-PCR analyses of various differentiation markers for the three germ layers.

FIG. 38 shows directed differentiations of human iPS cells. (A) Phase contrast image of differentiated iPS cells after 18 days cultivation on PA6. (B) Immunocytochemistry of the cells shown in A with βIII-tubulin (red) and tyrosine hydroxylase (green) antibodies. Nuclei were stained with Hoechst 33342 (blue). (C) RT-PCR analyses of dopaminergic neuron markers. (D) Phase contrast image of iPS cells differentiated into cardiomyocytes. (E) RT-PCR analyses of cardiomyocyte markers. Bars=200 μm (A, D) and 100 μm (B).

FIG. 39 shows hematoxylin and eosin staining of teratoma derived from human iPS cells (clone 201B7).

FIG. 40 shows human iPS cells (phase contrast images) derived from fibroblast-like synoviocytes (HFLS, clone 243H1) and BJ fibroblasts (clone 24601). Bars=200 μm.

FIG. 41 shows expression of ES cell marker genes in iPS cells derived from HFLS and BJ fibroblasts.

FIG. 42 shows embryoid body-mediated differentiation of iPS cells derived from HFLS and BJ fibroblasts.

FIG. 43 shows the effect of family factors and the omission of Myc on generation of iPS cells from Nanog-reporter MEFs. (A) Generation of iPS cells with family genes from MEF by Nanog selection. The number of GFP-positive colonies is shown. The results of three independent experiments were shown with different colors (white, gray, and black). The “4 factors” indicate the combination of Oct3/4, Sox2, Klf4, and c-Myc. (B) The effect of puromycin selection timing on iPS cell generation. Shown are GFP-positive colonies observed 28 days after the transduction of the four factors or the three factors devoid of Myc. (C) The effect of puromycin selection timing on the percentage of GFP-positive colonies per all colonies.

FIG. 44 shows teratomas derived from iPS cells, which were induced from Fbx15-reporter MEFs with family proteins.

FIQ. 45 shows characterization of iPS cells induced from Nanog-reporter MEFs without Myc retroviruses. RT-PCR showing expression levels of ES cell marker genes and the four factors. By using specific primer sets, total transcripts, transcripts from the endogenous genes (endo), and the transcripts from the retroviruses (Tg) were distinguished for the four factors.

FIG. 46 shows generation of iPS cells without Myc retroviruses from MEFs containing the Fbx15-reporter and the constitutively active GFP-transgene. (A) Morphology of iPS cells generated without Myc retroviruses. The bar indicates 500 μm. (B) RT-PCR analyses of ES marker genes in ES, MEF, and iPS cells induced without Myc. (C) Chimeras derived from iPS cells induced without Myc (clones 142B-6 and -12).

FIG. 47 shows the efficient isolation of iPS cells without drug selection. (A) Morphology of iPS cells induced from adult TTF containing the Nanog-GFP-IRES-Puro^{r} reporter. Cells were transduced with either the four factors or the three factors devoid of Myc, together with DsRed, and then were cultured for 30 days without drug selection. The expression of the Nanog reporter (Nanog-GFP) and the DsRed retrovirus (Tg-DsRed) was examined by fluorescent microscopy. The bar indicates 500 μm. (B) Morphology of iPS cells induced from adult TTF, which contained a DsRed transgene driven by a constitutively active promoter (ACTB, β-actin gene), but lacking the Nanog- or Fbx15-selection cassettes. The cells were transduced with either the four factors or the three factors devoid of Myc, together with GFP, and then cultured for 30 days without drug selection. The expression of the GFP retrovirus (Tg-GFP) was examined by fluorescent microscopy. The bar indicates 500 μm. (C) RT-PCR analyses of ES maker genes in iPS cells generated from TTF without drug selection and ES cells. (D) Chimeras derived from iPS cells, which were generated from adult TTF without drug selection or the Myc retroviruses.

FIG. 48 shows induction of human iPS cells without Myc retroviruses. (A) The retroviruses for Oct3/4, Sox2 and Klf4 were introduced into BJ fibroblasts (246G) or HDF (253G). After 30 days, a few hES cell-like colonies emerged. These cells were expandable and showed hES cell-like morphology. (B) The expression of ES cell marker genes in human iPS cells derived from HDF without Myc retroviruses (253G) or with Myc (253F). (C) Embryoid body-mediated differentiation of human iPS cells generated without Myc retroviruses.

FIG. 49 shows results from experiments using six factors and two different combinations of four factors. The vertical axis shows the number of colonies. The term “6F” refers to the six factors (klf4, c-myc, oct3/4, sox2, nanog and Lin-28), the term “Y4F” refers to the first combination of four factors (Klf4, c-myc, oct3/4 and sox2), and the term “T4F” refers to the second combination of four factors (oct3/4, sox2, nanog and Lin-28), respectively. The term “ES like” refers to ES-like cell colony morphologically, and the term “total” shows total number of ES-like cell colonies and non-ES like cell colonies. Exp #1, Exp #2, Exp #3, and Exp #4 show individual experimental results, respectively.

FIG. 50 shows a summary of data from experiments performed with mouse embryonic fibroblasts (MEFs). (A) 1.0×10⁵ MEF cells obtained from Nanog GFP^{tg/-}Fbx15^{-/-} mouse were seeded on gelatin coated 6 well plates. Next day, four factors (Oct3/4, Klf4, Sox2, c-Myc) or three factors (Oct3/4, Klf4, Sox2) were retrovirally transduced into the cells. After 4 days of the infection, cells were re-seeded 1 to 2 or 1 to 6-ratio on 6 well plates covered with mitomycin C-treated STO cells. Drug selection was started at 14 days or 21 days. At day 28, GFP positive cells were counted and cells were stained for alkaline phosphatase (AP) and crystal violet (CV). (B) Summary of the number of the GFP positive colonies from three independent experiments, Exp. #1, 2, and 3. (C) Percentage of GFP positive colonies from three independent iPS experiments, Exp. #1, 2, and 3.

FIG. 51 shows a summary of data from experiments performed with adult human dermal fibroblasts. 1.0×10⁵ adult HDF cells expressing slc7a were seeded on 6 well plates. Next day, four factors (Oct3/4, Klf4, Sox2, c-Myc) or three factors (Oct3/4, Klf4, Sox2) were retrovirally transduced into the cells. After 6 days of the infection, 5.0×10⁵ cells were re-seeded on 100 mm plates covered with 1.5×10⁶ of mitomycin C-treated STO cells. At day 7 the medium was replaced with Primate ES cell medium supplemented with 4 ng/ml bFGF. This figure shows colony numbers at 30 days after infections.

FIG. 52 shows the number of colonies confirmed on the 32^{nd} day (d32; left bar) and the 40^{th} day (d40; right bar) after the infection in which 4 factors (Y4F, i.e., OCT3/4, SOX2, KLF4, c-MYC) or 3 factors (Y3F, i.e., OCT3/4, SOX2, KLF4) derived from human, and either mouse Sall4 (mSall4), mousy Sall1 (mSall1) or both mSall4 and Sall1 were transduced with retroviruses into adult human dermal fibroblasts (HDFs) expressing Slc7a. “+Mock” shows the group in the case where a retrovirus derived from a vector containing neither mSall4 nor Sall1 together with Y3F or Y4F were transduced; “+mSall4” shows the group in the case where mSall4 together with Y3F or Y4F were transduced; “+Sall1” shows the group in the case where Sall1 together with Y3F or Y4F were transduced; and “+mSall4+mSall1” shows the group in the case where both of mSall4 and mSall1 together with Y3F or Y4F were transduced. The vertical axis shows the number of human ES-like colonies confirmed on a 10 cm culture plate. The same experiment was repeated three times. The total number of colonies of each experiment is shown above each bar of the graph.

FIG. 53 shows the number of colonies confirmed on the 32^{nd} day (d32; left bar) and the 40^{th} day (d40; right bar) after the infection in which (A) 3 factors (T3F, i.e., OCT3/4, SOX2, NANOG) or (8) 4 factors (T4F, i.e., OCT3/4, SOX2, NANOG, LIN-28) derived from human, and either mouse Sall4 (mSall4), mouse Sall1 (mSall1) or both mSall4 and mSall1 were transduced with retroviruses into adult human dermal fibroblasts (HDFs) expressing Slc7a. “+Mock” shows the group in the case where a retrovirus derived from a vector containing neither mSall4 nor Sall1 together with T3F or T4F were transduced; “+mSall4” shows the group in the case where mSall4 together with T3F or T4F were transduced; “+mSall1” shows the group in the case where mSall1 together with T3F or T4F were transduced; and “+mSall4+Sall1” shows the group in the case where both mSall4 and mSall1 together with T3F or T4F were transduced. Vertical axes show the number of human ES-like colonies confirmed on a 10 cm culture plate. The same experiment was repeated twice. The total number of colonies of each experiment is shown above each bar of the graph.

FIG. 54 shows the number of colonies confirmed on the 32^{nd} day (d32; left bar) and the 40^{th} day (d40; right bar) after the infection in which (A) 3 factors (Y3F, i.e., OCT3/4, SOX2, KLF4) or (B) 4 factors (Y4F, i.e., OCT3/4, SOX2, KLF4, c-MYC) derived from human, and human SALL4 (hSALL4) were transduced with retroviruses into adult human dermal fibroblasts (HDFs) expressing Slc7a. “+Mock” shows the group in the case where a retrovirus derived from a vector containing no hSALL4 together with Y3F or Y4F were transduced; “+hSALL4” shows the group in the case where hSALL4 together with Y3F or Y4F were transduced. Vertical axes show the number of human ES like colonies confirmed on a 10 cm culture plate. The same experiment was repeated twice or three times. The total number of colonies of each experiment is shown above each bar of the graph.

FIG. 55 is a graph which shows the result of counting of the colony number of human iPS cells which were established by transducing 4 genes (OCT3/4, KLF4, and SOX2 and any one of c-MYC, L-MYC or N-MYC) into aHDF-Slc7a1 by retrovirus. The values on each graph show the ratio of the iPS colony number to total colony number.

FIG. 56 is a photograph which shows the result of staining of cells differentiated from human iPS cells using an anti-smooth muscle actin antibody, anti-a-fetoprotein antibody or anti-βIII-tubulin antibody. It was confirmed that the human iPS cells (32R2 and 32R6) which were established by transducing 4 genes (OCT3/4, SOX2, KLF4, and L-MYC) into aHDF-Slc7a1 by retrovirus have an ability to differentiate into three germ layers.

FIG. 57 shows tissue-stained images (hematoxylin-eosin staining) of teratomas which were obtained by injecting the human iPS cell (32R6) which was established by transduction of 4 genes (OCT3/4, SOX2, KLF4, and L-MYC) into a testis of SCID mouse. The upper row of images shows the tissue image of nerve tissue (A), bowel-like tissue (B), and cartilage tissue (C), respectively, from the left. The lower row of images shows the tissue image of hair tissue (D), adipose tissue (E), and pigment tissue (F), respectively, from the left.

FIG. 58 is a photograph which shows the morphology of sixteen GFP-positive colonies (iPS-MEF-Ng-443-3) which were obtained by transducing 3 genes (Oct3/4, Klf4, and L-Myc) into MEF-Ng by retrovirus. The upper row of images shows GFP-positive colonies, and the lower row of images shows phase-contrast images.

FIG. 59 is a photograph which shows the result of RT-PCR and genomic PCR using a clone derived from a GFP-positive colony (iPS-MEF-Ng-443-3). “Total” shows the result of detection of expression of endogenous gene and retrovirus-derived gene using specific primers for each factor. “Tg” shows the result of detection of expression of retrovirus-derived gene using specific primers for each factor. “Genome” shows the result of detection of presence of retrovirus-derived gene integrated into the genome DNA using specific primers for each factor. The numbers on the right side of the photograph indicate the cycle number of PCR. In the figure, “RF8” shows the result of the similar experiment using ES cell as a control, “20D17” shows the result of the similar experiment using Nanog-iPS (Okita et al. Nature 448:313-17, 2007), and “142E-9” shows the result of the similar experiment using iPS cell (Fbx-iPS) which was obtained by transducing 4 genes (Oct3/4, Klf4, L-Myc, and Sox2) into MEF-Fbx. In the figure, “Plasmid” shows the result obtained by using plasmid of each factor (pMXs-Sox2, pMXs-Oct3/4, pMXs-Klf4, pMXs-c-Myc, and pMXs-L-Myc).

FIG. 60 is a photograph which shows the result of staining of cells differentiated from mouse iPS cells using an anti-smooth muscle actin antibody, anti-α-fetoprotein antibody or anti-βIII-tubulin antibody. It was confirmed that the mouse iPS cells (443-3-3, 443-3-6, 443-3-12, and 443-3-13) which were established by transduction of 3 genes (Oct3/4, Klf4 and L-Myc) have an ability to differentiate into three germ layers. RF8 is a control ES cell.

FIG. 61 is a tissue-stained image (hematoxylin-eosin staining) of teratoma which was obtained by subcutaneously injecting the mouse iPS cells (443-3-3, 443-3-6, 443-3-12, and 443-3-13) which were established by transduction of 3 genes (Oct3/4, Klf4, and L-Myc) into a nude mouse. The figure shows the tissue image of nerve tissue, bowel-like tissue, muscle tissue, epidermal tissue and cartilage tissue, respectively, from the top.

FIG. 62 is a graph which shows the results of counting the number of colonies of human iPS cells which were established by transducing 3 genes (OCT3/4, KLF4, and SOX2), and the c-MYC, L-MYC and/or LIN28 gene into aHDF-Slc7a1 cells by retrovirus. The black bar shows the total number of colonies, and the white bar shows the total number of iPS colonies.

FIG. 63 schematically shows the structure of various Myc chimera genes and mutated genes.

FIG. 64 shows the results of counting the number of colonies of human iPS cells which were established by transducing various Myc chimera genes and mutated genes into aHDF-Slc7a1 cells by retrovirus. FIG. 64A shows the number of hiPS cell colonies; FIG. 64B shows the total number of colonies; and FIG. 64C shows the ratio (%) of the iPS cell colony number to the total colony number.

### DETAILED DESCRIPTION OF THE PREFERRED EMBODIMENT

Various investigations were conducted to address the aforementioned need for pluripotent stem cells which can be derived from a patient's own somatic cells, and for nuclear reprogramming factors capable of generating pluripotent stem cells from somatic cells. Investigations were also conducted to identify nuclear reprogramming factors by using the screening method for a nuclear reprogramming factor disclosed in International Publication WO2005/80598. While International Publication WO 2005/80598 discloses a screening method, this document fails to disclose any nuclear reprogramming factor. Furthermore, this document fails to specify any nuclear reprogramming factor or candidate nuclear reprogramming factor which would be capable of generating an induced pluripotent stem cell.

Ultimately, 24 kinds of candidate genes were found as genes relating to nuclear reprogramming, and among them, three kinds of the genes were found as particularly preferred for nuclear reprogramming: sometimes these genes are referred to as essential in certain embodiments. As further discussed throughout the specification, the nuclear reprogramming factor of the present invention may contain one or more factors relating to differentiation, development, proliferation or the like and factors having other physiological activities, as well as other gene products which can function as a nuclear reprogramming factor. The present invention was achieved on the basis of these findings.

The present invention provides at least the following advantages and features: induced pluripotent stem (iPS) cells derived by nuclear reprogramming of a somatic cell, including methods for reprogramming of a differentiated cell without using eggs, embryos, or embryonic stem (ES) cells.

As further discussed herein with respect to the general guidance for the reprogramming of differentiated cells and the examples, the present invention also provides various nuclear reprogramming factors capable of generating pluripotent stem cells from somatic cells. The nuclear reprogramming factor may comprise one or more gene products. The nuclear reprogramming factor may also comprise a combination of gene products. Each nuclear reprogramming factor may be used alone or in combination with other nuclear reprogramming factors as disclosed herein. In addition, nuclear reprogramming may be performed with small molecules, compounds, or other agents such that iPS cells are obtained.

In a preferred embodiment, the nuclear reprogramming factor comprises a gene product of each of the following three kinds of genes: an Oct family gene, a Klf family gene, and a Sox family gene. According to a more preferred embodiment of the invention, there is provided the aforementioned factor comprising a gene product of each of the following three kinds of genes: Oct3/4, Klf4, and Sox2.

In another embodiment of the invention, there is provided a nuclear reprogramming factor comprising a gene product of each of the following three kinds of genes: an Oct family gene, a Klf family gene, and a Myc family gene. According to a preferred embodiment of the invention, there is provided the aforementioned factor comprising a gene product of each of the following three kinds of genes: Oct3/4, Klf4 and c-Myc.

According to another preferred embodiment, there is provided the aforementioned factor, which further comprises a gene product of the following gene: a Sox family gene, and as a more preferred embodiment, there is provided the aforementioned factor, which comprises a gene product of Sox2.

According to still another preferred embodiment, there is provided the aforementioned factor, which comprises a cytokine together with the gene product of the Myc family gene, or alternatively, instead of the gene product of the Myc family gene. As a more preferred embodiment, there is provided the aforementioned factor, wherein the cytokine is basic fibroblast growth factor (bFGF) and/or stem cell factor (SCF). Accordingly, it is understood that the nuclear reprogramming factor can be with or without the Myc family gene.

According to particularly preferred embodiments, there is provided a nuclear reprogramming factor for a somatic cell, which comprises a gene product of the TERT gene in addition to a gene product of each of an Oct family gene, a Klf family gene, a Myc family gene, and a Sox family gene; and the aforementioned factor, which comprises a gene product or gene products of one or more kinds of genes selected from the group consisting of the following genes: SV40 Large T antigen (SEQ ID NO: 23), HPV16 E6 (SEQ ID NO: 24), HPV16 E7 (SEQ ID NO: 25), and Bmil, in addition to a gene product of each of the Oct family gene, the Klf family gene, the Myc family gene, the Sox family gene, and the TERT gene.

In addition to these factors, there is provided the aforementioned factor, which further comprises a gene product or gene products of one or more kinds of genes selected from the group consisting of the following: Fbx15, Nanog, ERas, ECAT15-2, Tell, and β-catenin.

There is also provided the aforementioned factor, which comprises a gene product or gene products of one or more kinds of genes selected from the group consisting of the following: ECAT1, Esg1, Dnmt3L, ECAT8, Gdf3, Sox15, ECAT15-1, Fth117, Sall4, Rex1, UTF1, Stella, Stat3, and Grb2.

The present invention also provides a nuclear reprogramming factor comprising a gene product or gene products of one or more kinds of the following genes: Oct3/4, Sox2, Klf4, Nanog, Lin-28, and c-Myc.

The present invention also provides a nuclear reprogramming factor comprising any combination of gene products, small molecules and/or substances as described herein, further comprising one or more factors improving the efficiency of iPS cell induction, such as one or more gene products of a Sall1 and/or Sall4 gene, a Myc gene (including c-Myc, N-Myc, and/or L-Myc, as well as mutants and chimeras thereof), and/or a Lin28 gene. The one or more gene products capable of improving iPS cell induction, such as a Sall1, Sall4, Myc, Lin28, and/or Nanog gene may be derived from a mammal, e.g., mouse, rat, or human.

In another aspect, the present invention provides a method for preparing an induced pluripotent stem cell by nuclear reprogramming of a somatic cell, which comprises the step of contacting the aforementioned nuclear reprogramming factor with the somatic cell.

There is also provided the aforementioned method, which comprises the step of adding the aforementioned nuclear reprogramming factor to a culture of the somatic cell; the aforementioned method, which comprises the step of introducing a gene encoding the aforementioned nuclear reprogramming factor into the somatic cell; the aforementioned method, which comprises the step of introducing said gene into the somatic cell by using a recombinant vector containing at least one kind of gene encoding the aforementioned nuclear reprogramming factor; and the aforementioned method, wherein a somatic cell isolated from a patient is used as the somatic cell.

In another aspect, the present invention provides an induced pluripotent stem cell obtained by the aforementioned method. The present invention also provides a somatic cell derived by inducing differentiation of the aforementioned induced pluripotent stem cell.

The present invention further provides a method for stem cell therapy, which comprises the step of transplanting a somatic cell, wherein said cell is obtained by inducing differentiation of an induced pluripotent stem cell obtained by the aforementioned method using a somatic cell isolated and collected from a patient, into said patient. Several kinds of, preferably approximately 200 kinds of iPS cells prepared from somatic cells derived from healthy humans can be stored in an iPS cell bank as a library of iPS cells, and one kind or more kinds of the iPS cells in the library can be used for preparation of somatic cells, tissues, or organs that are free of rejection by a patient to be subjected to stem cell therapy.

The present invention further provides a method for evaluating a physiological function or toxicity of a compound, a medicament, a poison or the like by using various cells obtained by inducing differentiation of an induced pluripotent stem cell obtained by the aforementioned method.

The present invention also provides a method for improving ability of differentiation and/or growth of a cell, which comprises the step of contacting the aforementioned nuclear reprogramming factor with the cell, and further provides a cell obtained by the aforementioned method, and a somatic cell derived by inducing differentiation of a cell obtained by the aforementioned method.

By using the nuclear reprogramming factor provided by the present invention, reprogramming of a differentiated cell nucleus can be conveniently and highly reproducibly induced without using embryos or ES cells, and an induced pluripotent stem cell, as an undifferentiated cell having differentiation ability, pluripotency, and growth ability similar to those of ES cells, can be established. For example, an induced pluripotent stem cell having high growth ability and differentiation pluripotency can be prepared from a patient's own somatic cell by using the nuclear reprogramming factor of the present invention. Cells obtainable by differentiating said cell (for example, cardiac muscle cells, insulin producing cells, nerve cells and the like) are extremely useful, because they can be utilized for stem cell transplantation therapies for a variety of diseases such as cardiac insufficiency, insulin dependent diabetes mellitus, Parkinson's disease and spinal cord injury, thereby the ethical problem concerning the use of human embryo and rejection after transplantation can be avoided. Further, various cells obtainable by differentiating the induced pluripotent stem cell (for example, cardiac muscle cells, hepatic cells and the like) are highly useful as systems for evaluating efficacy or toxicity of compounds, medicaments, poisons and the like.

As noted above, transplantation of ES cells has a problem of causing rejection in the same manner as organ transplantation. Moreover, from an ethical viewpoint, there are many dissenting opinions against the use of ES cells, which are established by destroying human embryos.

The present invention provides at least the following advantages and features:

### Identification of Nuclear Reprogramming Factors

As will be further disclosed below, the nuclear reprogramming factor of the present invention may contain one or more factors relating to differentiation, development, proliferation or the like and factors having other physiological activities, as well as other gene products which can function as a nuclear reprogramming factor. It is understood that such embodiments fall within the scope of the present invention, and the present invention is, in other words, directed to factors inducing pluripotent stem cells and various methods of obtaining induced pluripotent stem cells, including various manners of reprogramming differentiated cells as well as various manners of culturing, maintaining, and differentiating the induced pluripotent stem cells.

Furthermore, by using somatic cells in which only one or two genes among the three kinds of genes Oct3/4, Klf4, and c-Myc are expressed, other gene products which can function as a nuclear reprogramming factor can be identified by, for example, performing screening for a gene product which can induce nuclear reprogramming of said cells. For example, depending on the kinds of genes expressed in a differentiated cell, one or more genes useful as a reprogramming factor can be determined using the guidance herein provided. According to the present invention, the aforementioned screening method is also provided as a novel method for screening for a nuclear reprogramming factor. In other words, the present invention is not limited to any particular combination of nuclear reprogramming factors and the nuclear reprogramming factors of the present invention can be identified by screening methods, for example, the aforementioned screening method.

In one embodiment, the nuclear reprogramming factor of the present invention is characterized in that it comprises one or more gene products. As a means for confirming the nuclear reprogramming factor of the present invention, for example, the screening method for nuclear reprogramming factors disclosed in International Publication WO 2005/80598 can be used. The entire disclosure of the aforementioned publication is incorporated into the disclosure of the specification by reference. By referring to the aforementioned publication, those skilled in the art can perform screening of nuclear reprogramming factors to confirm the existence and the action of the reprogramming factor of the present invention.

For example, as an experimental system allowing for observation of the reprogramming phenomenon, a mouse can be used in which the βgeo (a fusion gene of the β galactosidase gene and the neomycin resistance gene) is knocked into the Fbx15 locus (Tokuzawa et al. Mal. Cell Biol. 23:2699-708, 2003). The details are described in the examples of the specification. The mouse Fbx15 gene is a gene specifically expressed in differentiation pluripotent cells such as ES cells and early embryos. In a homomutant mouse in which βgeo is knocked into the mouse Fbx15 gene so as to be deficient in the Fbx15 function, abnormal phenotypes including those relating to differentiation pluripotency or generation are not generally observed. In this mouse, the expression of the βgeo is controlled by the enhancer and promoter of the Fbx15 gene, and differentiated somatic cells in which βgeo is not expressed have sensitivity to G418. In contrast, βgeo knockin homomutant ES cells have resistance against G418 at an extremely high concentration (higher than 12 mg/ml). By utilizing this phenomenon, an experimental system can be constructed to visualize reprogramming of somatic cells.

By applying the aforementioned experimental system, fibroblasts (Fbx15^{βgeo/βgeo} MEFs) can be first isolated from an embryo of the βgeo knockin homomutant mouse (13.5 days after fertilization). The MEFs do not express the Fbx15 gene, and accordingly also do not express geo to give sensitivity to G418. However, when the MEFs are fused with genetic manipulation-free ES cells (also have sensitivity to G418), βgeo is expressed and the cells become G418-resistant as a result of reprogramming of nuclei of MEFs. Therefore, by utilizing this experimental system, the reprogramming phenomenon can be visualized as G418 resistance.

Nuclear reprogramming factors can be selected by using the aforementioned experimental system. As candidates of genes relevant to nuclear reprogramming factors, a plurality of genes can be selected which show specific expression in ES cells or of which important roles in the maintenance of pluripotency of ES cells are suggested, and it can be confirmed whether or not each candidate gene can induce nuclear reprogramming alone or in an appropriate combination thereof. For example, a combination of all of the selected primary candidate genes is confirmed to be capable of inducing the reprogramming of differentiated cells into a state close to that of ES cells. Combinations are then prepared by withdrawing each individual gene from the aforementioned combination, and the same actions of the combinations are confirmed in order to select each secondary candidate gene whose absence causes a reduction of the reprogramming induction ability or loss of the reprogramming induction ability. By repeating similar steps for the secondary candidate genes selected as described above, an essential combination of nuclear reprogramming genes can be selected, and it can be confirmed that a combination of gene products of each of the three kinds of genes, e.g., an Oct family gene, a Klf family gene, and a Myc family gene, acts as a nuclear reprogramming factor. It can be further confirmed that a combination of a gene product of a Sox family gene additionally with the gene products of the aforementioned three kinds of genes has extremely superior characteristics as a nuclear reprogramming factor. Specific examples of the selection method for the nuclear reprogramming factors are demonstrated in the examples of the specification.

Therefore, by referring to the above general explanations and specific explanations of the examples, those skilled in the art can readily confirm that the combination of these three kinds of genes induces the reprogramming of somatic cells, and that the combination of these three kinds of gene products is essential for nuclear reprogramming in certain embodiments. Thus, the embodiments herein illustrate various combinations of gene products and/or nuclear reprogramming factors which can provide iPS cells. In other words, based on the disclosure provided herein, one of ordinary skill in the art would know from the disclosed examples and/or readily determine which combination and/or combinations of nuclear reprogramming factors, including gene products, can generate pluripotent stem cells.

### Nuclear Reprogramming Factor (NRF)

In a preferred embodiment, the NRF comprises a gene product. The nuclear reprogramming factor can be used to induce reprogramming of a differentiated cell without using eggs, embryos, or ES cells, to conveniently and highly reproducibly establish an induced pluripotent stem cell having pluripotency and growth ability similar to those of ES cells. For example, the nuclear reprogramming factor can be introduced into a cell by transducing the cell with a recombinant vector comprising a gene encoding the nuclear reprogramming factor. Accordingly, the cell can express the nuclear reprogramming factor expressed as a product of a gene contained in the recombinant vector, thereby inducing reprogramming of a differentiated cell.

The nuclear reprogramming factor may comprise a protein or peptide. The protein may be produced from the aforementioned gene, or alternatively, in the form of a fusion gene product of said protein with another protein, peptide or the like. The protein or peptide may be a fluorescent protein and/or a fusion protein. For example, a fusion protein with green fluorescence protein (GFP) or a fusion gene product with a peptide such as a histidine tag can also be used. Further, by preparing and using a fusion protein with the TAT peptide derived form the virus HIV, intracellular uptake of the nuclear reprogramming factor through cell membranes can be promoted, thereby enabling induction of reprogramming only by adding the fusion protein to a medium thus avoiding complicated operations such as gene transduction. Since preparation methods of such fusion gene products are well known to those skilled in the art, skilled artisans can easily design and prepare an appropriate fusion gene product depending on the purpose.

Nuclear reprogramming may also be accomplished with one or more small molecules, compounds, including inorganic and organic compounds, or mixtures thereof, extracts, epigenetic factors, and/or other components of the cytoplasm of a pluripotent cell.

In a particularly preferred embodiment, the nuclear reprogramming factor may comprise one or more gene products of each of the following three kinds of genes: an Oct family gene, a Klf family gene, and a Sox family gene.

In another preferred embodiment, the nuclear reprogramming factor may comprise one or more gene products of each of: an Oct family gene, a Klf family gene, and a Myc family gene.

The nuclear reprogramming factor may also comprise one or more gene products of each of: an Oct family gene, a Klf family gene, a Myc family gene, and a Sox family gene.

The nuclear reprogramming factor may also comprise one or more gene products of each of: an Oct family gene, a Klf family gene, and a cytokine. [none exemplary embodiment, the above-referenced nuclear reprogramming factor may further comprise one or more gene products of a Myc family gene. In another exemplary embodiment, the above referenced nuclear reprogramming factor may further comprise one or more gene products of a Sox family gene.

The cytokines of the present invention are not particularly limited. For example, the cytokine may comprise basic fibroblast growth factor (bFGF/FGF2) or stem cell factor (SCF).

With regard to gene family members, the nuclear reprogramming factor may comprise any combination of members from one or more gene families. For example, a combination of one or more gene products of Oct3/4, Klf4, and c-Myc. Examples of the Oct family gene include, for example, Oct3/4, Oct IA, Oct6, and the like. Oct3/4 is a transcription factor belonging to the POU family, and is reported as a marker of undifferentiated cells (Okamoto et al. Cell 60:461-72, 1990). Oct3/4 is also reported to participate in the maintenance of pluripotency (Nichols et al. Cell 95:379-91, 1998). Examples of the Klf family gene include Klf1, Klf2, Klf4, Klf5 and the like. Klf4 (Kruppel like factor-4) is reported as a tumor repressing factor (Ghaleb et al. Cell Res. 15:92-96, 2005). Examples of the Myc family gene include c-Myc, N-Myc, L-Myc and the like. c-Myc is a transcription control factor involved in differentiation and proliferation of cells (Adhikary & Eilers, Nat. Rev. Mol. Cell Biol. 6:635-45, 2005), and is also reported to be involved in the maintenance of pluripotency (Cartwright et al. Development 132:885-96, 2005). L-Myc, which as noted is a Myc family member, was revealed to have much lower transformation activity than c-Myc, i.e., 1% to 10% of the transformation activity of c-Myc (Birrer et al. Molecular and Cellular Biology 8 (6): 2668-2673, 1988); Barrett et al. Molecular and Cellular Biology 12 (7): 3130-3137, 1992, both of which are hereby incorporated by reference in their entireties). The NCBI accession numbers of the genes of the families other than Oct3/4, Klf4 and c-Myc are set in TABLE 1 as follows:

TABLE 1

| | | Mouse | Human |
|---|---|---|---|
| Klf1 | Kruppel-like factor 1 (erythroid) | NM_010635 | NM_006563 |
| Klf2 | Kruppel-like factor 2 (lung) | NM_008452 | NM_016270 |
| Klf5 | Kruppel-like factor 5 | NM_009769 | NM_001730 |
| c-Myc | myelocytomatosis oncogene | NM_010849 | NM_002467 |
| N-Myc | v-Myc myelocytomatosis viral | NM_008709 | NM_005378 |
| | related oncogene, neuroblastoma | | |
| | derived (avian) | | |
| L-Myc | v-Myc myelocytomatosis viral | NM_008506 | NM_005376 |
| | oncogene homolog 1, lung | | |
| | carcinoma derived (avian) | | |
| Oct1A | POU domain, class 2, transcription | NM_198934 | NM_002697 |
| | factor 1 | | |
| Oct6 | POU domain, class 3, transcription | NM_011141 | NM_002699 |
| | factor 1 | | |

All of these genes are those commonly existing in mammals including human, and for use of the aforementioned gene products in the present invention, genes derived from arbitrary mammals (those derived from mammals such as mouse, rat, bovine, ovine, horse, and ape) can be used. In addition to wild-type gene products, mutant gene products including substitution, insertion, and/or deletion of several (for example, 1 to 10, preferably 1 to 6, more preferably 1 to 4, still more preferably 1 to 3, and most preferably 1 or 2) amino acids and having similar function to that of the wild-type gene products can also be used. For example, as a gene product of c-Myc, a stable type product (T58A) may be used as well as the wild-type product. The above explanation may be applied similarly to the other gene products.

The nuclear reprogramming factor of the present invention may comprise a gene product other than the aforementioned three kinds of gene products. An example of such gene product includes a gene product of a Sox family gene. Examples of the Sox family genes include, for example, Sox1, Sox3, Sox7, Sox15, Sox17 and Sox18, and a preferred example includes Sox2. A nuclear reprogramming factor comprising at least a combination of the gene products of four kinds of genes, an Oct family gene (for example, Oct3/4), a Klf family gene (for example, Klf4), a Myc family gene (for example, c-Myc), and a Sox family gene (for example, Sox2) is a preferred embodiment of the present invention from a viewpoint of reprogramming efficiency, and in particular, a combination of a gene product of a Sox family gene is sometimes preferred to obtain pluripotency. Sox2, expressed in an early development process, is a gene encoding a transcription factor (Avilion et al. Genes Dev. 17:126-40, 2003). The NCBI accession numbers of Sox family genes other than Sox2 are in TABLE 2 as follows.

TABLE 2

| | | Mouse | Human |
|---|---|---|---|
| Sox1 | SRY-box containing gene 1 | NM_009233 | NM_005986 |
| Sox3 | SRY-box containing gene 3 | NM_009237 | NM_005634 |
| Sox7 | SRY-box containing gene 7 | NM_011446 | NM_031439 |
| Sox15 | SRY-box containing gene 15 | NM_009235 | NM_006942 |
| Sox17 | SRY-box containing gene 17 | NM_011441 | NM_022454 |
| Sox18 | SRY-box containing gene 18 | NM_009236 | NM_018419 |

Further, a gene product of a Myc family gene may be replaced with a cytokine. As the cytokine, for example, SCF, bFGF or the like is preferred. However, cytokines are not limited to these examples.

The nuclear reprogramming factor of the present invention may comprise a gene product of a Lin family gene. Examples of the Lin family genes include, for example, Lin28 and Lin28b, and a preferred example includes Lin28. The NCBI accession numbers of Lin family genes are as follows:
Lin28: NM_145833 (mouse), NM_024674 (human); and
Lin28b: NM_001031772 (mouse), NM_001004317 (human).

As a more preferred embodiment, an example includes a factor which induces immortalization of cells, in addition to the aforementioned three kinds of gene products, preferably, the four kinds of gene products. For example, an example includes a combination of a factor comprising a gene product of the TERT gene. In another exemplary embodiment, the nuclear reprogramming factor comprises any of the aforementioned gene products in combination with a factor comprising a gene product or gene products of one or more kinds of the following genes: SV40 Large T antigen, HPV16 E6, HPV16 E7, and Bmil. TERT is essential for the maintenance of the telomere structure at the end of chromosome at the time of DNA replication, and the gene is expressed in stem cells or tumor cells in humans, whilst it is not expressed in many somatic cells (Horikawa et al. P.N.A.S. USA 102:18437-442, 2005). SV40 Large T antigen, HPV16 E6, HPV16 E7, or Bmil was reported to induce immortalization of human somatic cells in combination with Large T antigen (Akimov et al. Stem Cells 23:1423-33, 2005; Salmon et al. Mol. Ther. 2:404-14, 2000). These factors are extremely useful particularly when iPS cells are induced from human cells, The NCBI accession numbers of TERT and Bmi1 genes are listed in TABLE 3 as follows.

TABLE 3

| | | Mouse | Human |
|---|---|---|---|
| TERT | telomerase reverse | NM_009354 | NM_198253 |
| | transcriptase | | |
| Bmi1 | B lymphoma Mo-MLV | NM_007552 | NM_005180 |
| | insertion region 1 | | |

Furthermore, a gene product or gene products of one or more kinds of genes selected from the group consisting of the following: Fbx15, Nanog, ERas, ECAT15-2, Tel1, and B-catenin may be combined. As a particularly preferred embodiment from a viewpoint of reprogramming efficiency, an example includes a nuclear reprogramming factor comprising a total of ten kinds of gene products, wherein gene products of Fbx15, Nanog, ERas, ECAT15-2, Tel1, and β-catenin are combined with the aforementioned four kinds of gene products. Fbx15 (Tokuzawa et al. Mol. Cell Biol. 23:2699-708, 2003), Nanog (Mitsui et al. Cell 113:631-42, 2003), ERas (Takahashi et al. Nature 423:541-45, 2003), and ECAT15-2 (Bortvin et al. Development 130:1673-80, 2003) are genes specifically expressed in ES cells. Tcl1 is involved in the activation of Akt (Bortvin et al. Development 130:1673-80, 2003), and B-catenin is an important factor constituting the Wnt signal transmission pathway, and also reported to be involved in the maintenance of pluripotency (Sato et al. Nat. Med. 10:55-63, 2004).

Further, the nuclear reprogramming factor of the present invention may comprise, for example, a gene product or gene products of one or more kinds of genes selected from the group consisting of the following: ECAT1, Esg1, Dnmt3L, ECAT8, Gdf3, Sox15, ECAT15-1, Fth117, Sall4, Rex1, UTF1, Stella, Stat3, and Grb2. ECAT1, Esg1, ECAT8, Gdf3, and ECAT15-1 are genes specifically expressed in ES cells (Mitsui et al. Cell 113:631-42, 2003). Dnmt3L is a DNA methylating enzyme-related factor, and Sox15 is a class of genes expressed in an early development process and encoding transcription factors (Maruyama et al. J Biol. Chem. 280:24371-79, 2005). Fth117 encodes ferritin heavy polypeptide-like 17 (colLoriot et al. Int. J Cancer 105:371-76, 2003), Sall4 encodes a Zn finger protein abundantly expressed in embryonic stem cells (Kohlhase et al. Cytogenet. Genome Res. 98:274-77, 2002), and Rex1 encodes a transcription factor locating downstream from Oct3/4 (Ben-Shushan et al. Mol. Cell Biol. 18:1866-78, 1998). UTF1 is a transcription cofactor locating downstream from Oct3/4, and it is reported that the suppression of the proliferation of ES cells is induced when this factor is suppressed (Okuda et al. EMBO J. 17:2019-32, 1998). Stat3 is a signal factor for proliferation and differentiation of cells. The activation of Stat3 triggers the operation of UF, and thereby the factor plays an important role for the maintenance of pluripotency (Niwa et al. Genes Dev. 12:2048-60, 1998). Grb2 encodes a protein mediating between various growth factor receptors existing in cell membranes and the Ras/MAPK cascade (Cheng et al. Cell 95:793-803, 1998).

However, as noted above, the gene products which may be included in the nuclear reprogramming factor of the present invention are not limited to the gene products of the genes specifically explained above. The nuclear reprogramming factor of the present invention may contain one or more factors relating to differentiation, development, proliferation or the like and factors having other physiological activities, as well as other gene products which can function as a nuclear reprogramming factor. It is understood that such embodiments fall within the scope of the present invention. By using somatic cells in which only one or two genes among the three kinds of the gene Oct3/4, Klf4, and c-Myc are expressed, the other gene products which can function as a nuclear reprogramming factor can be identified by, for example, performing screening for a gene product which can induce nuclear reprogramming of said cells. According to the present invention, the aforementioned screening method is also provided as a novel method for screening for a nuclear reprogramming factor.

Examples of nuclear reprogramming factors containing an Oct family factor, a Klf family factor, and a Myc family factor include:
(1) a combination comprising Oct3/4, Klf4, and L-Myc;
(2) a combination comprising Oct 3/4, Klf4, L-Myc, and Sox2; and
(3) a combination comprising Oct 3/4, Klf4, L-Myc, Sox2, and Lin28.

While any number of combinations of gene products or factors can be used to establish iPS cells, some combinations of factors have been found to establish iPS cells with higher efficiency than others. For example, a higher efficiency of iPS cell establishment can be achieved by using L-Myc instead of c-Myc. In particular, human iPS cell can be established with higher efficiency when using L-Myc instead of c-Myc. Accordingly, it is preferred to use L-Myc for the preparation of iPS cells, especially human iPS cells.

Furthermore, the combinations of factors are not limited to the use of wild-type genes or gene products. For example, Myc chimeras or other Myc variants can be used instead of wild-type Myc. In particular, examples of Myc chimeras or variants include Ms-cL-Myc, Ms-cLc-Myc as shown in Example 29, and human homologs thereof. Other examples of Myc variants include mutants of c-Myc comprising nucleic acid and/or amino acid deletions, substitutions, or additions.

Human homologs of mouse chimeric Myc or mouse Myc variants can be readily prepared by one of ordinary skill in the art on the basis of the information of the nucleotide and amino acid sequences of human c-Myc (GenBank Acc. No. NM_002467) and human L-Myc (GenBank Acc. No. NM_005376), as well as that of the nucleotide and amino acid sequences of mouse Myc chimeras and/or mouse Myc variants disclosed in the instant specification.

### Cells of the Invention and Methods of Generating the Same

By using the nuclear reprogramming factor of the present invention, the nucleus of a somatic cell can be reprogrammed to obtain an induced pluripotent stem cell. In the specification, the term “induced pluripotent stem cells” means cells having properties similar to those of ES cells, and more specifically, the term encompasses undifferentiated cells having pluripotency and growth ability. However, the term should not be construed narrowly in any sense, and should be construed in the broadest sense. The method for preparing induced pluripotent stem cells by using a nuclear reprogramming factor is explained in International Publication WO2005/80598 (the term “ES-like cells” is used in the publication), and a means for isolating induced pluripotent stem cells is also specifically explained. Therefore, by referring to the aforementioned publication, those skilled in the art can easily prepare induced pluripotent stem cells by using the nuclear reprogramming factor of the present invention. Methods for preparing induced pluripotent stem cells from somatic cells by using the nuclear reprogramming factor of the present invention are not particularly limited. Any method may be employed as long as the nuclear reprogramming factor can contact with somatic cells under an environment in which the somatic cells and induced pluripotent stem cells can proliferate. An advantage of the present invention is that an induced pluripotent stem cell can be prepared by contacting a nuclear reprogramming factor with a somatic cell in the absence of eggs, embryos, or embryonic stem (ES) cells.

For example, a gene product contained in the nuclear reprogramming factor of the present invention may be added to a medium. Alternatively, by using a vector containing a gene that is capable of expressing the nuclear reprogramming factor of the present invention, a means of transducing said gene into a somatic cell may be employed. When such vector is used, two or more kinds of genes may be incorporated into the vector, and each of the gene products may be simultaneously expressed in a somatic cell. When one or more of the gene products contained in the nuclear reprogramming factor of the present invention are already expressed in a somatic cell to be reprogrammed, said gene products may be excluded from the nuclear reprogramming factor of the present invention. It is understood that such embodiments fall within the scope of the present invention.

Specific means for using a retrovirus as a vector is disclosed in WO 2007/69666; Takahashi et al. Cel/126:663-676, 2006; and Takahashi et al. Cell 131:861-872, 2007, which are herein incorporated by reference in their entireties. Specific means for using a lentivirus as a vector is disclosed in Yu et al. Science 318:1917-1920, 2007, which is herein incorporated by reference in its entirety. Specific means for using adenovirus as a vector is disclosed in Stadtfeld et al. (Science published online: Sep. 25, 2008, 10.1126/science.1162494), which is herein incorporated by reference in its entirety. Specific means for using a plasmid as a non-viral vector is disclosed in Okita et al. (Science, published online: Oct. 9, 2008, 10.1126/science. 1164270), which is herein incorporated by reference in its entirety. One of ordinary skill in the art could choose and use an appropriate means from among the above known means, or from any of the other known means available in the prior art.

As indicated above, the nuclear reprogramming factor of the present invention can be used to generate iPS cells from differentiated adult somatic cells. In the preparation of induced pluripotent stem cells by using the nuclear reprogramming factor of the present invention, types of somatic cells to be reprogrammed are not particularly limited, and any kind of somatic cells may be used. For example, matured somatic cells may be used, as well as somatic cells of an embryonic period. Other examples of cells capable of being generated into iPS cells and/or encompassed by the present invention include mammalian cells such as fibroblasts, B cells, T cells, dendritic cells, ketatinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, pancreatic cells, hepatocytes, macrophages, monocytes, mononuclear cells, and gastric cells, including gastric epithelial cells. The cells can be embryonic, or adult somatic cells, differentiated cells, cells with an intact nuclear membrane, non-dividing cells, quiescent cells, terminally differentiated primary cells, and the like.

Induced pluripotent stem cells may express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECATS, E-cadherin; f.3111-tubulin; a-smooth muscle actin (a-SMA); fibroblast growth factor 4 (Fgf4), Cripto, Daxl; zinc finger protein 296 (Zfp296); N-acetyltransferase-1 (Natl); (ES cell associated transcript 1 (ECAT1); ESG1/DPPAS/ECAT2; ECAT3; ECAT6; ECAT7; ECAT8; ECAT9; ECAT10; ECAT15-1; ECAT15-2; Fth117; Sall4; undifferentiated embryonic cell transcription factor (UTF1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, Fox03; GDF3; CYP25A1; developmental pluripotency-associated 2 (DPPA2); T-cell lymphoma breakpoint 1 (Tel1); OPPA3/Stella; OPPA4; other general markers for pluripotency, etc. Other markers can include Dnmt3L; Sox15; Stat3; Grb2; SV40 Large T Antigen; HPV16 E6; HPV 16 E7, β-catenin, and Bmi1. Such cells can also be characterized by the down-regulation of markers characteristic of the differentiated cell from which the iPS cell is induced. For example, iPS cells derived from fibroblasts may be characterized by down-regulation of the fibroblast cell marker Thy1 and/or up-regulation of SSEA-1. It is understood that the present invention is not limited to those markers listed herein, and encompasses markers such as cell surface markers, antigens, and other gene products including ESTs, RNA (including microRNAs and antisense RNA), DNA (including genes and cDNAs), and portions thereof.

When induced pluripotent stem cells are used for therapeutic treatment of diseases, it is desirable to use somatic cells isolated from patients. For example, somatic cells involved in diseases, somatic cells participating in therapeutic treatment of diseases and the like can be used. A method for selecting induced pluripotent stem cells that appear in a medium according to the method of the present invention is not particularly limited, and a well-known means may be suitably employed, for example, a drug resistance gene or the like can be used as a marker gene to isolate induced pluripotent stem cells using drug resistance as an index. Various media that can maintain undifferentiated state and pluripotency of ES cells and various media which cannot maintain such properties are known in this field, and induced pluripotent stem cells can be efficiently isolated by using a combination of appropriate media. Differentiation and proliferation abilities of isolated induced pluripotent stem cells can be easily confirmed by those skilled in the art by using confirmation means widely applied to ES cells.

Thus, another preferred embodiment of the invention comprises a pluripotent stem cell induced by reprogramming a somatic cell in the absence of eggs, embryos, or embryonic stem-(ES) cells. The pluripotent stem cell can be a mammalian cell, for example a mouse, human, rat, bovine, ovine, horse, hamster, dog, guinea pig, or ape cell. For example, direct reprogramming of somatic cells provides an opportunity to generate patient- or disease-specific pluripotent stem cells. Mouse iPS cells are indistinguishable from ES cells in morphology, proliferation, gene expression, and teratoma formation. Furthermore, when transplanted into blastocysts, mouse iPS cells can give rise to adult chimeras, which are competent for germline transmission (Maherali et al. Cell Stem Cell 1:55-70, 2007; Okita et al. Nature 448:313-17, 2007; Wemig et al. Nature 448:318-324, 2007). Human iPS cells are also expandable and indistinguishable from human embryonic stem (ES) cells in morphology and proliferation. Furthermore, these cells can differentiate into cell types of the three germ layers in vitro and in teratomas.

The present invention also provides for the generation of somatic cells derived by inducing differentiation of the aforementioned pluripotent stem cells. The present invention thus provides a somatic cell derived by inducing differentiation of the aforementioned induced pluripotent stem cell.

In another embodiment, there is disclosed a method for improving differentiation ability and/or growth ability of a cell, which comprises contacting a nuclear reprogramming factor with a cell.

In a particularly preferred embodiment, the present invention comprises a method for stem cell therapy comprising: (1) isolating and collecting a somatic cell from a patient; (2) inducing said somatic cell from the patient into an iPS cell; (3) inducing differentiation of said iPS cell, and (4) transplanting the differentiated cell from step (3) into the patient.

In another preferred embodiment, the present invention includes a method for evaluating a physiological function of a compound comprising treating cells obtained by inducing differentiation of an induced pluripotent stem cell with the compound.

A method for evaluating the toxicity of a compound comprising treating cells obtained by inducing differentiation of an induced pluripotent stem cell in the presence of the compound.

### Examples

Various terms, abbreviations and designations for the raw materials and tests used in the following Examples are explained as follows:

### Abbreviations

iPS cell (induced pluripotent stem cell)
NRF (nuclear reprogramming factor)
ES cell (embryonic stem cell)
TTF (tail tip fibroblast)
MEF (mouse embryonic fibroblast)
HOF (human dermal fibroblast)
bFGF (basic fibroblast growth factor)
SCF (stem cell factor)
GFP (green fluorescent protein)

The present invention will be more specifically explained with reference to examples. However, the scope of the present invention is not limited to the following examples.

### Example 1: Selection of a Nuclear Reprogramming Factor

In order to identify reprogramming factors, an experimental system for easy observation of the reprogramming phenomenon is required. As an experimental system, a mouse in which βgeo (a fusion gene of P-galactosidase gene and neomycin resistance gene) was knocked into the Fbx15 locus (Tokuzawa et al. Mot. Cell Biol. 23:2699-708, 2003) was used. The mouse Fbx15 gene is a gene specifically expressed in differentiation pluripotent cells such as ES cells and early embryos. However, in a homomutant mouse in which βgeo was knocked into the mouse Fbx15 gene so as to delete the function of Fbx15, no abnormal phenotypes including those concerning differentiation pluripotency or development were observed. In this mouse, expression control of βgeo is attained by the enhancer and promoter of the Fbx15 gene. Specifically, βgeo is not expressed in differentiated somatic cells, and they have sensitivity to G418. In contrast, the βgeo knockin homomutant ES cells have resistance against G418 at an extremely high concentration (higher than 12 mg/ml). By utilizing the above phenomenon, an experimental system for visualizing the reprogramming of somatic cells was constructed.

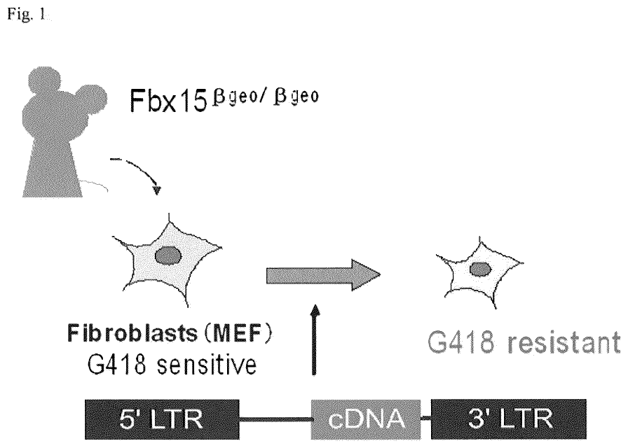

In the aforementioned experimental system, fibroblasts (Fbx15^{βgeo/βgeo} MEFs) were first isolated from an embryo of the βgeo knockin homomutant mouse (13.5 days after fertilization). Since MEFs do not express the Fbx15 gene, the cells also do not express βgeo and thus have sensitivity to G418. Whilst when the MEFs are fused with ES cells that have not been gene-manipulated (also having sensitivity to G418), the nuclei of MEFs are reprogrammed, and as a result, βgeo is expressed to give G418-resistance. The reprogramming phenomenon can thus be visualized as G418 resistance by using this experimental system (International Publication W02005/80598). Searches for reprogramming factors were performed by using the aforementioned experimental system (FIG. 1), and total 24 kinds of genes were selected as candidate reprogramming factors, including genes showing specific expression in ES cells and genes suggested to have important roles in the maintenance of differentiation pluripotency of ES cells. These genes are shown in TABLES 4 and 5 below. For β-catenin (#21) and c-Myc (#22), active type mutants (catenin: S33Y, c-Myc: T58A) were used.

TABLE 4

| Number | Name of Gene | Explanation of Gene |
|---|---|---|
| 1 | ECAT1 | ES cell associated transcript 1 (ECAT1) |
| 2 | ECAT2 | developmental pluripotency associated 5 |
| | | (DPPA5), ES cell specific gene 1 (ESG1) |
| 3 | ECAT3 | F-box protein 15 (Fbx15), |
| 4 | ECAT4 | homeobox transcription factor Nanog |
| 5 | ECAT5 | ES cell expressed Ras (ERas) |
| 6 | ECAT7 | DNA (cytosine-5-)-methyltransferase |
| | | 3-like (Dnmt3l), valiant 1 |
| 7 | ECAT8 | ES cell associated transcript 8 (ECAT8) |
| 8 | ECAT9 | growth differentiation factor 3 (Gdf3) |
| 9 | ECAT10 | SRY-box containing gene 15 (Sox15) |
| 10 | ECAT15-1 | developmental pluripotency associated 4 |
| | | (Dppa4), variant 1 |
| 11 | ECAT15-2 | developmental pluripotency associated 2 |
| | | (Dppa2) |
| 12 | Fthl17 | ferritin, heavy polypeptide-like 17 (Fthl17) |
| 13 | Sall4 | sal-like 4 (Drosophila) (Sall4), transcript |
| | | variant a |
| 14 | Oct3/4 | POU domain, class 5, transcription factor 1 |
| | | (Pou5f1) |
| 15 | Sox2 | SRY-box containing gene 2 (Sox2) |
| 16 | Rex1 | zinc finger protein 42 (Zfp42) |
| 17 | Utf1 | undifferentiated embryonic cell transcription |
| | | factor 1 (Utf1) |
| 18 | Tcl1 | T-cell lymphoma breakpoint 1 (Tcl1) |
| 19 | Stella | developmental pluripotency-associated 3 |
| | | (Dppa3) |
| 20 | Klf4 | Kruppel-like factor 4 (gut) (Klf4) |
| 21 | β-catenin | catenin (cadherin associated protein), beta 1, |
| | | 88kDa (Ctnnb1) |
| 22 | c-Myc | myelocytomatosis oncogene (Myc) |
| 23 | Stat3 | signal transducer and activator of |
| | | transcription 3 (Stat3), transcript |
| | | variant 1 |
| 24 | Grb2 | growth factor receptor bound protein 2 (Grb2) |

TABLE 5
NCBI accession number

| | Name | | | |
|---|---|---|---|---|
| Number | of Gene | Characteristic Feature | Mouse | Human |
| 1 | ECAT1 | Gene specifically expressed in ES | AB211060 | AB211062 |
| | | cell | | |
| 2 | ECAT2 | Gene specifically expressed in ES | NM_025274 | NM_001025290 |
| | | cell | | |
| 3 | ECAT3 | Gene specifically expressed in ES | NM_015798 | NM_152676 |
| | | cell | | |
| 4 | ECAT4 | Transcription factor having | AB093574 | NM_024865 |
| | | homeodomain, essential factor for | | |
| | | differentiation pluripotency | | |
| | | maintenance | | |
| 5 | ECAT5 | Ras family protein, ES cell growth | NM_181548 | NM_181532 |
| | | promoting factor | | |
| 6 | ECAT7 | DNA methylation enzyme-related | NM_019448 | NM_013369 |
| | | factor, essential for imprinting | | |
| 7 | ECAT8 | Gene specifically expressed in ES | AB211061 | AB211063 |
| | | cell, having Tudor domain | | |
| 8 | ECAT9 | Gene specifically expressed in ES | NM_008108 | NM_020634 |
| | | cell, belonging to TGFβ family | | |
| 9 | ECAT10 | Gene specifically expressed in ES | NM_009235 | NM_006942 |
| | | cell, SRY family transcription factor | | |
| 10 | ECAT15-1 | Gene specifically expressed in ES | NM_028610 | NM_018189 |
| | | cell | | |
| 11 | ECAT15-2 | Gene specifically expressed in ES | NM_028615 | NM_138815 |
| | | cell | | |
| 12 | Fthl17 | Gene specifically expressed in ES | NM_031261 | NM_031894 |
| | | cell, similar to ferritin heavy chain | | |
| 13 | Sall4 | Gene specifically expressed in ES | NM_175303 | NM_020436 |
| | | cell, Zn finger protein | | |
| 14 | Oct3/4 | POU family transcription factor, | NM_013633 | NM_002701 |
| | | essential for pluripotency | | |
| | | maintenance | | |
| 15 | Sox2 | SRY family transcription factor, | NM_011443 | NM_003106 |
| | | essential for pluripotency | | |
| | | maintenance | | |
| 16 | Rex1 | Gene specifically expressed in ES | NM_009556 | NM_174900 |
| | | cell, Zn finger protein | | |
| 17 | Utf1 | Transcription regulation factor | NM_009482 | NM_003577 |
| | | highly expressed in ES cell, | | |
| | | promoting growth of ES | | |
| 18 | Tcl1 | Oncogene activating AKT, | NM_009337 | NM_021966 |
| | | abundantly expressed in ES cell | | |
| 19 | Stella | Gene specifically expressed in ES | NM_139218 | NM_199286 |
| | | cell | | |
| 20 | Klf4 | Abundantly expressed in ES cell, | NM_010637 | NM_004235 |
| | | both actions as antioncogene and | | |
| | | oncogene are reported | | |
| 21 | β-catenin | Transcription factor activated by | NM_007614 | NM_001904 |
| | | Wnt signal, involvement in | | |
| | | pluripotency maintenance is reported | | |
| 22 | c-Myc | Transcription control factor | NM_010849 | NM_002467 |
| | | participating in cell proliferation and | | |
| | | differentiation and oncogene, | | |
| | | involvement in pluripotency | | |
| | | maintenance is reported | | |
| 23 | Stat3 | Transcription factor activated by LIF | NM_213659 | NM_139276 |
| | | signal, considered essential for | | |
| | | pluripotency maintenance of mouse | | |
| | | ES cells | | |
| 24 | Grb2 | Adapter protein mediating growth | NM_008163 | NM_002086 |
| | | factor receptors and Ras/MAPK | | |
| | | cascade | | |

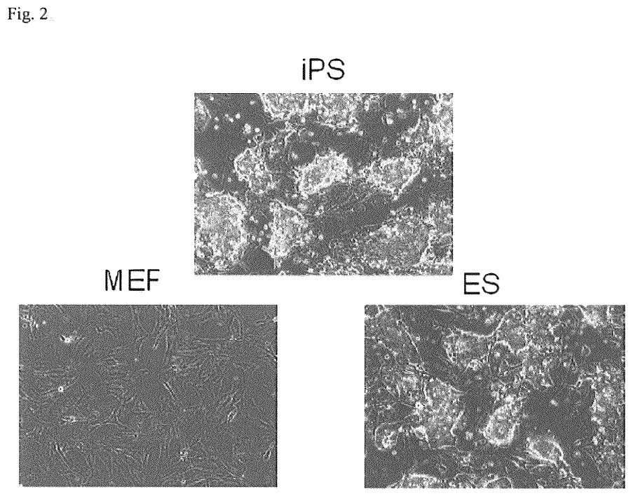

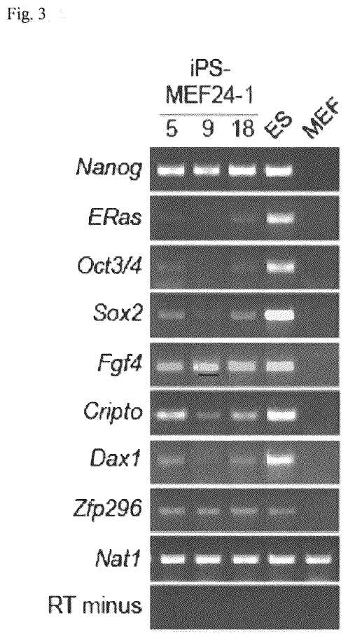

cDNAs of these genes were inserted into the retroviral vector pMX-gw by the Gateway technology. First, each of the 24 genes was infected into Fbx15^{βgeo/βgeo} MEFs, and then G418 selection was performed under ES cell culture conditions. However, no G418-resistant colony was obtained. Next, the retroviral vectors of all of the 24 genes were simultaneously infected into Fbx15^{βgeo/βgeo} MEFs. When G418 selection was performed under ES cell culture conditions, a plurality of drug resistant colonies were obtained. These colonies were isolated, and cultivation was continued. It was found that cultivation of these cells over a long period of time could be performed, and that these cells had morphology similar to that of ES cells (FIG. 2). In the figure, iPS cells represent induced pluripotent stem cells (also called “ES like cells”, “ES-like cells”, or “ESL cells”), ES represents embryonic stem cells, and MEF represents differentiated cells (embryonic fibroblasts).

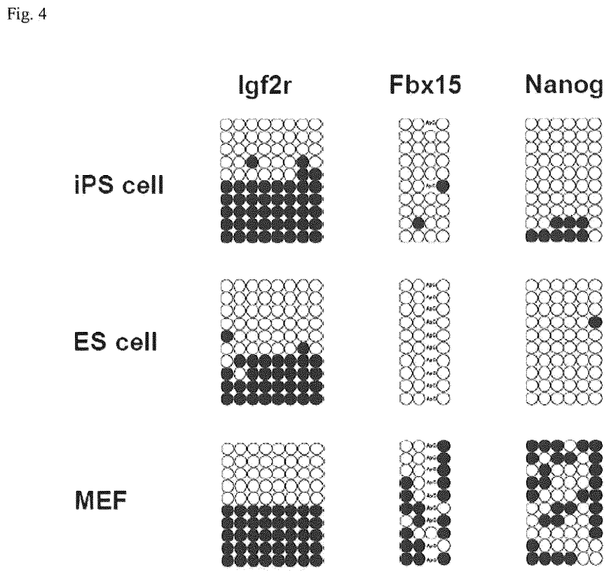

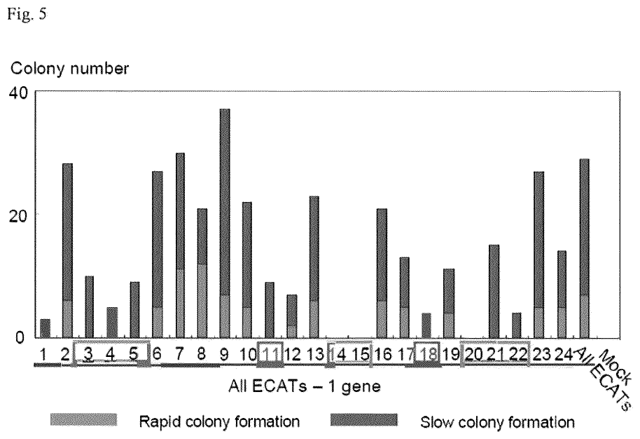

When expression profiles of the marker genes were examined by RT-PCR, undifferentiation markers such as Nanog and Oct3/4 were found to be expressed (FIG. 3). It was found that the expression of Nanog was close to that of ES cells, whereas the expression of Oct3/4 was lower than that of ES cells. When DNA methylation status was examined by the bisulfite genomic sequencing, it was found that the Nanog gene and Fbx15 gene were highly methylated in MEFs, whereas they were demethylated in the iPS cells (FIG. 4). About 50% of IGF2 gene, an imprinting gene, was methylated both in the MEF and iPS cells. Since it was known that the imprinting memory was deleted and the IGF2 gene was almost completely demethylated in the primordial germ cells at 13.5 days after fertilization, from which the Fbx15^{βgeo/βgeo} MEFs were isolated, it was concluded that iPS cells were not derived from primordial germ cells contaminated in the Fbx15^{βgeo/βgeo} MEFs. The above results demonstrated that reprogramming of the differentiated cells (MEFs) into a state close to that of ES cells was able to be induced with the combination of the 24 kinds of factors.

Then, studies were made as to whether or not all of the 24 kinds of genes were required for the reprogramming. With withdrawal of each individual gene, 23 genes were transfected into the Fbx15^{βgeo/βgeo} MEFs. As a result, for 10 genes, colony formation was found to be inhibited with each withdrawal thereof (FIG. 5, the gene numbers correspond to the gene numbers shown in TABLE 4, and the genes are the following 10 kinds of genes: #3, #4, #5, #11, #14, #15, #18, #20, #21, and #22). When these ten genes were simultaneously transfected into the Fbx15^{βgeo/βgeo} MEFs, G418-resistant colonies were significantly more efficiently obtained as compared to simultaneous transfection with the 24 genes.

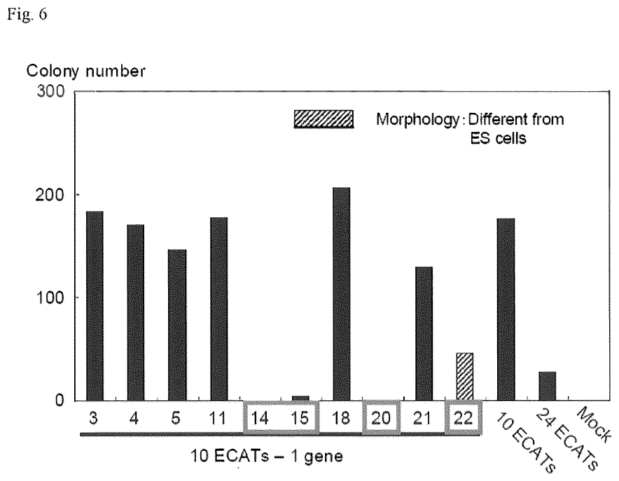

Furthermore, 9 genes, with withdrawal of each individual gene from the 10 genes, were transfected into Fbx15^{βgeo/βgeo} MEFs. As a result, it was found that G418-resistant iPS cell colonies were not formed when each of 4 kinds of genes (#14, #15, #20, or #22) was withdrawn (FIG. 6). Therefore, it was suggested that these four kinds of genes, among the ten genes, had particularly important roles in the induction of reprogramming.

Example 2: Induction of Reprogramming with a Combination of 4 Kinds of Genes

It was examined whether or not induction of reprogramming of somatic cells was achievable with the four kinds of genes of which particular importance was suggested among the 10 genes. By using the combination of the aforementioned 10 kinds of genes, the combination of the aforementioned 4 kinds of genes, combinations of only 3 kinds of genes among the 4 kinds of genes, and combinations of only 2 kinds of genes among the 4 kinds of genes, these sets of genes were retrovirally transduced into the MEF cells as somatic cells in which βgeo was knocked into the Fbx15 gene. As a result, when the 4 kinds of genes were transduced, 160 G418-resistant colonies were obtained. Although this result was almost the same as that obtained by the transduction with the 10 kinds of genes (179 colonies), the colonies obtained by the 4-gene transduction were smaller than those by the 10-gene transduction. When these colonies were passaged, the numbers of colonies having iPS cell morphology was 9 clones among 12 clones in the case of the 10-gene transduction, whereas there was a somewhat lower tendency of 7 clones among 12 clones in the case of the 4-gene transduction. As for the 4 genes, almost the same numbers of iPS cells were obtained with either of those derived from mouse or those derived from human.

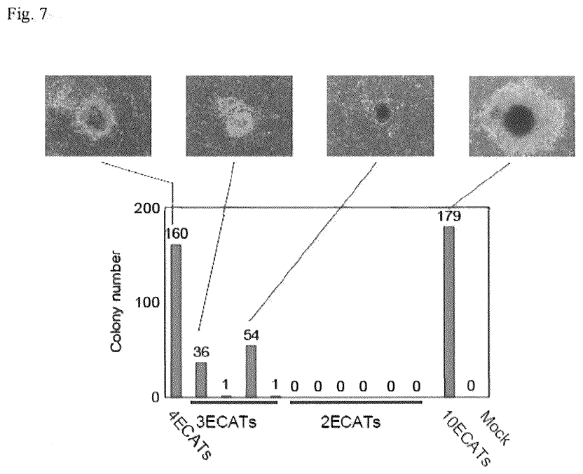

When 3 genes selected from the aforementioned 4 genes were transduced, 36 flat colonies were obtained with one combination (#14 (Oct3/4), #15 (Sox2), and #20 (Klf4)). However, iPS cells were not observed when they were passaged. With another combination (#14 (Oct3/4), #20 (Klf4), and #22 (c-Myc)), 54 small colonies were obtained. When 6 of the relatively large colonies from among those colonies were passaged, cells similar to ES cells were obtained for all these 6 clones. However, it seemed that adhesion of the cells between themselves and to the culture dish was weaker than that of ES cells. The proliferation rate of the cells was also slower than that observed in the case of the transduction with the 4 genes. Further, one colony each was formed with each of the other two kinds of combinations of 3 genes among the 4 genes. However, proliferation of the cells was not observed when the cells were passaged. With any of combinations of 2 genes selected from the 4 genes (6 combinations), no G418-resistant colonies were formed. The above results are shown in FIG. 7.

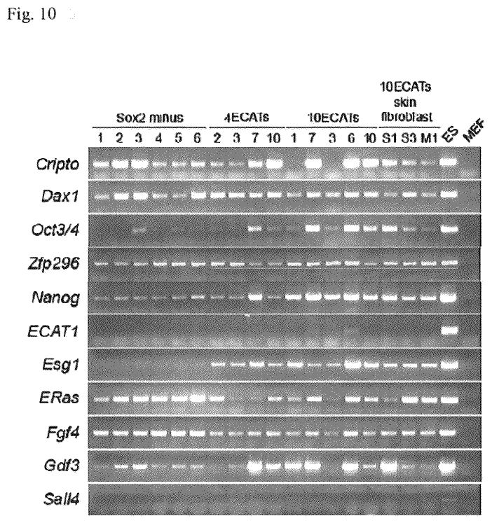

Further, the results of observation of expression profiles of the ES cell marker genes by RT-PCR are shown in FIG. 10. The details of the method are as follows. From iPS cells established by transducing 3 genes (Oct3/4, Klf4, and c-Myc: represented as “Sox2 minus”), 4 genes (Sox2 was added to the three genes: represented as “4ECAT”), and 10 genes (#3, #4, #5, #11, #18, and #21 in TABLE 4 were added to the four genes: represented as “10ECAT”) into Fbx15^{βgeo/βgeo} MEFs; iPS cells established by transducing 10 genes into fibroblasts established from tail tip of an adult mouse in which βgeo was knocked into the Fbx15 gene (represented as “10ECAT Skin fibroblast”), mouse ES cells, and MEF cells with no gene transduction, total RNAs were purified, and treated with DNaseI to remove contamination of genomic DNA. First strand cDNAs were prepared by a reverse transcription reaction, and expression profiles of the ES cell marker genes were examined by PCR. For Oct3/4, Nanog, and ERas, PCR was performed by using primers which only amplified a transcript product from an endogenous gene, not from the transduced retrovirus. The primer sequences are shown in TABLE 6.

TABLE 6

| | | |
|---|---|---|
| ECAT1 | ECAT1-RT-S | TGT GGG GCC CTG AAA GGC GAG CTG AGA T (SEQ ID NO: 1) |
| | ECAT1-RT-AS | ATG GGC CGC CAT ACG ACG ACG CTC AAC T (SEQ ID NO: 2) |
| Esg1 | pH34-U38 | GAA GTC TGG TTC CTT GGC AGG ATG (SEQ ID NO: 3) |
| | pH34-L394 | ACT CGA TAC ACT GGC CTA GC (SEQ ID NO: 4) |
| Nanog | 6047-S1 | CAG GTG TTT GAG GGT AGC TC (SEQ ID NO: 5) |
| | 6047-AS1 | CGG TTC ATC ATG GTA CAG TC (SEQ ID NO: 6) |
| ERas | 45328-S118 | ACT GCC CCT CAT CAG ACT GCT ACT (SEQ ID NO: 7) |
| | ERas-AS304 | CAC TGC CTT GTA CTC GGG TAG CTG (SEQ ID NO: 8) |
| Gdf3 | Gdf3-U253 | GTT CCA ACC TGT GCC TCG CGT CTT (SEQ ID NO: 9) |
| | GDF3 L16914 | AGC GAG GCA TGG AGA GAG CGG AGC AG (SEQ ID NO: 10) |
| Fgf4 | Fgf4-RT-S | CGT GGT GAG CAT CTT CGG AGT GG (SEQ ID NO: 11) |
| | Fgf4-RT-AS | CCT TCT TGG TCC GCC CGT TCT TA (SEQ ID NO: 12) |
| Cripto | Cripto-S | ATG GAC GCA ACT GTG AAC ATG ATG TTC GCA (SEQ ID NO: 13) |
| | Cripto-AS | CTT TGA GGT CCT GGT CCA TCA CGT GAC CAT (SEQ ID NO: 14) |
| Zfp296 | Zfp296-S67 | CCA TTA GGG GCC ATC ATC GCT TTC (SEQ ID NO: 15) |
| | Zfp296-AS350 | CAC TGC TCA CTG GAG GGG GCT TGC (SEQ ID NO: 16) |
| Dax1 | Dax1-S1096 | TGC TGC GGT CCA GGC CAT CAA GAG (SEQ ID NO: 17) |
| | Dax1-AS1305 | GGG CAC TGT TCA GTT CAG CGG ATC (SEQ ID NO: 18) |
| Oct3/4 | Oct3/4-S9 | TCT TTC CAC CAG GCC CCC GGC TC (SEQ ID NO: 19) |
| | Oct3/4-AS210 | TGC GGG CGG ACA TGG GGA GAT CC (SEQ ID NO: 20) |
| NAT1 | NAT1 U283 | ATT CTT CGT TGT CAA GCC GCC AAA GTG GAG (SEQ ID NO: 21) |
| | NAT1 L476 | AGT TGT TTG CTG CGG AGT TGT CAT CTC GTC (SEQ ID NO: 22) |

The results shown in this figure revealed that, by transduction of the 3 genes, expression of each of ERas and Fgf4 was efficiently induced, but expression of each of Oct3/4 and Nanog, essential factors for the maintenance of pluripotency, was not induced, or was very weak even when induced. However, when the 4 genes were transduced, there was one clone (#7) in which Oct3/4 and Nanog were relatively strongly induced among 4 clones examined. Further, when the 10 genes were transduced, strong induction of each of Oct3/4 and Nanog was observed in 3 clones among 5 clones examined.

These results revealed that a combination of at least 3 genes (#14 (Oct3/4), #20 (Klf4), and #22 (c-Myc)) was essential for reprogramming under these conditions, and in the cases of the 4-gene group and 10-gene group including the 3 kinds of genes, the reprogramming efficiency was increased in proportion to the increasing number of genes. In other words, in accordance with the guidance disclosed herein, the minimum combination of nuclear reprogramming factors required for iPS cell induction under a given set of experimental conditions could be further optimized as evidenced below.

### Example 3: Analysis of Pluripotency of Reprogrammed Cells

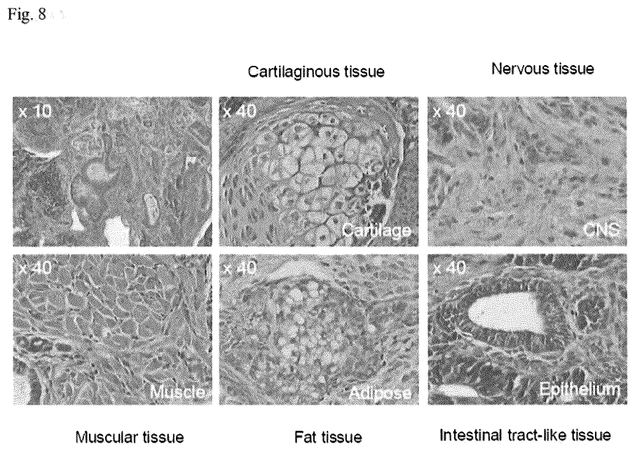

In order to evaluate the differentiation pluripotency of the established iPS cells, the iPS cells established with 24 factors, 10 factors, and 4 factors were subcutaneously transplanted into nude mice. As a result, tumors having a size similar to that observed with ES cells were formed in all animals. Histologically, the tumors consisted of a plurality of kinds of cells, and cartilaginous tissues, nervous tissues, muscular tissues, fat tissues, and intestinal tract-like tissues were observed (FIG. 8), which verified pluripotency of the iPS cells. In contrast, although tumors were formed when the cells established with the 3 factors were transplanted into nude mice, they were formed histologically only from undifferentiated cells. These results suggested that a Sox family gene was essential for the induction of differentiation pluripotency.

Example 4: Reprogramming of Fibroblasts Derived from Tails of Adult Mice

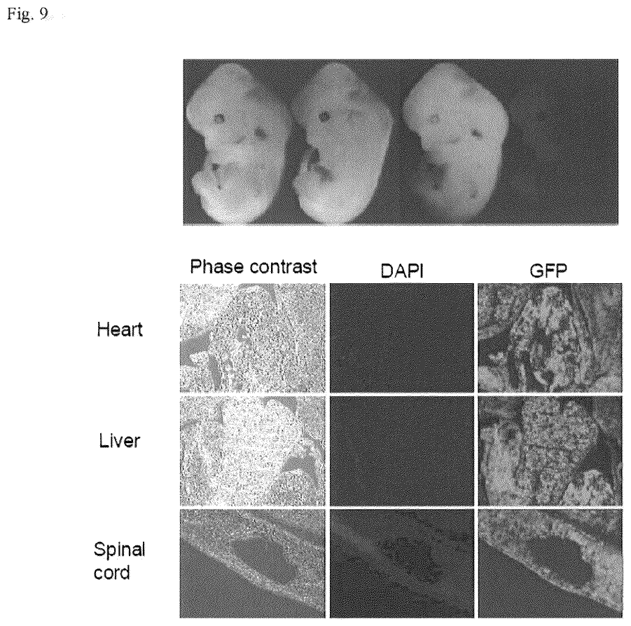

The 4 factors identified in the mouse embryonic fibroblasts (MEFs) were transduced into fibroblasts derived from tails of βgeo knockin Fbx15 adult mice systemically expressing green fluorescence protein (GFP). Then, the cells were cultured on feeder cells under the same conditions as ES cell culture conditions, and G418 selection was performed. In about two weeks after the start of the drug selection, a plurality of colonies of iPS cells were obtained. When these cells were subcutaneously transplanted to nude mice, teratomas consisting of a variety of all three germ layer tissues were formed. Further, when the iPS cells derived from adult dermal fibroblasts were transplanted to the blastocysts, and then transplanted into the uteri of pseudo-pregnant mice, embryos in which the GFP-positive cells were systemically distributed were observed among those at 13.5 days after fertilization (FIG. 9), demonstrating that the iPS cells had pluripotency and were able to contribute to mouse embryogenesis. These results indicate that the identified class of factors had an ability to induce reprogramming of not only somatic cells in an embryonic period, but also somatic cells of mature mice. Practically, it is extremely important that the reprogramming can be induced in cells derived from adult skin.

### Example 5: Effect of Cytokine on iPS Cell Establishment

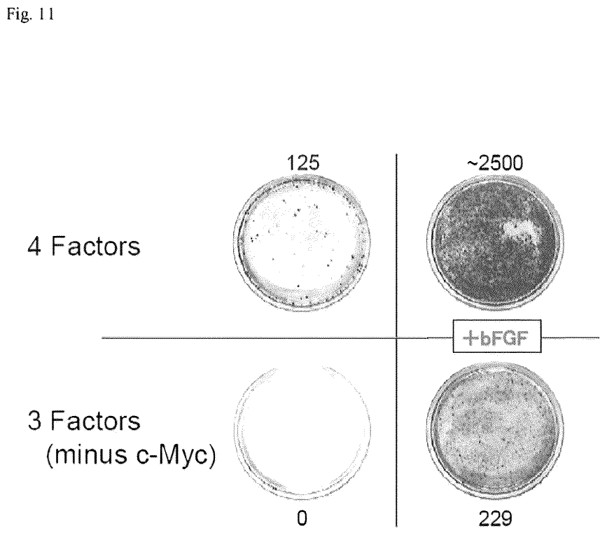

An effect of cytokine on iPS cell establishment was investigated. Expression vector (pMX retroviral vector) for basic fibroblast growth factor (bFGF) or stem cell factor (SCF) was transduced into feeder cells (STO cells) to establish cells permanently expressing the cytokines. MEFs derived from the Fbx15^{βgeo/βgeo} mouse (500,000 cells/100 mm dish) were cultured on these STO cells and transduced with the 4 factors, and then subjected to G418 selection. As a result, the number of formed colonies increased 20 times or higher on the STO cells producing bFGF (FIG. 11) or SCF (data not shown), as compared with the culture on normal STO cells. Further, although no iPS cell colony was formed on the normal STO cells when the 3 factors other than c-Myc were transduced, colony formation was observed on the STO cells producing bFGF (FIG. 11) or SCF (data not shown). These results revealed that stimulation with the cytokine increased the efficiency of the establishment of iPS cells from MEFs, and the nuclear reprogramming was achievable by using a cytokine instead of c-Myc.

Example 6: IPS Cell Generation with Other Oct, Klf, Myc, and Sox Family Members

Family genes exist for all of the Oct3/4, Klf4, c-Myc, and Sox2 genes (TABLES 1 and 2). Accordingly, studies were made as to whether iPS cells could be established with the family genes instead of the 4 genes. In TABLE 7, combined experimental results in duplicate are shown. With regard to the Sox family, Sox1 gave almost the same number of G418-resistant colonies formed and iPS cell establishment efficiency as those with Sox2. As for Sox3, the number of G418-resistant colonies formed was about 1/10 of that with Sox2, however, iPS cell establishment efficiency of the colonies picked up was in fact higher than that with Sox2. As for Sox15, both the number of G418-resistant colonies formed and iPS cell establishment efficiency were lower than those with Sox2. As for Sox17, the number of G418-resistant colonies formed was almost the same as that with Sox2, however, iPS cell establishment efficiency was low. With regard to the Klf family, Klf2 gave a smaller number of G418-resistant colonies than Klf4, however, they gave almost the same iPS cell establishment efficiency. With regard to the Myc family, it was found that wild-type c-Myc was almost the same as a T58A mutant both in the number of G418-resistant colonies formed and iPS cell establishment efficiency. Further, each of N-Myc and L-Myc (each wild type) was almost the same as c-Myc in both of the number of G418-resistant colonies formed and iPS cell establishment efficiency.

TABLE 7

| | Number | Number | Number of | |
|---|---|---|---|---|
| | of | of | established | iPS cell |
| Transduced | formed | picked | iPS | establishment |
| gene | colonies | colonies | cell strain | efficiency (%) |
| 4 Factors | 85 | 12 | 5 | 42 |
| (cMycT58A) | | | | |
| Sox1 | 84 | 12 | 7 | 58 |
| Sox3 | 8 | 8 | 7 | 92 |
| Sox15 | 11 | 11 | 1 | 8 |
| Sox17 | 78 | 12 | 2 | 17 |
| Klf2 | 11 | 10 | 5 | 50 |
| c-MycWT | 53 | 11 | 8 | 72 |
| N-MycWT | 40 | 12 | 7 | 58 |
| L-MycWT | 50 | 12 | 11 | 92 |
| 3 Factors | 6 | 6 | 2 | 17 |
| (-Sox2) | | | | |

### Example 7: Use of a Nanog-GFP-Puro^{r} Reporter to Establish iPS Cells

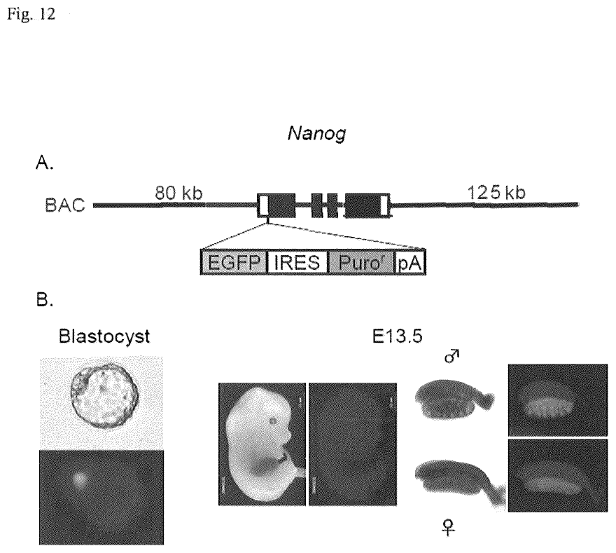

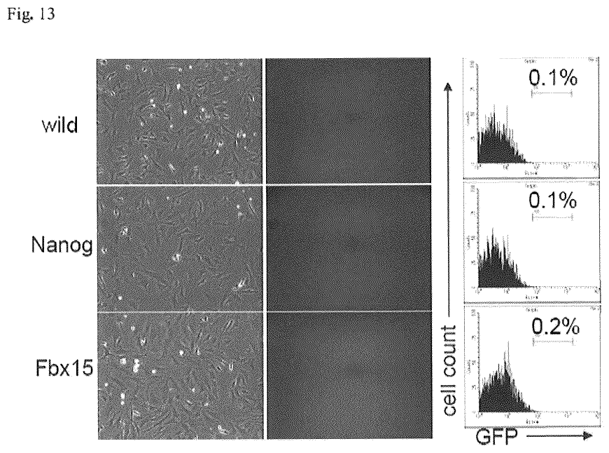

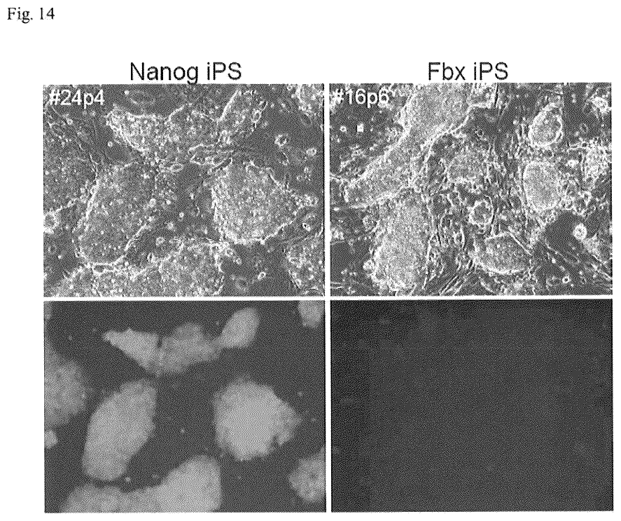

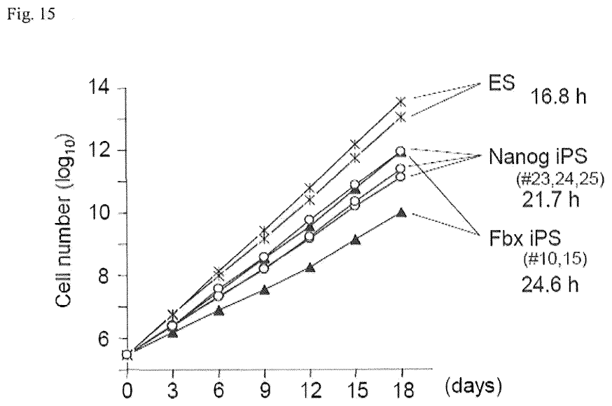

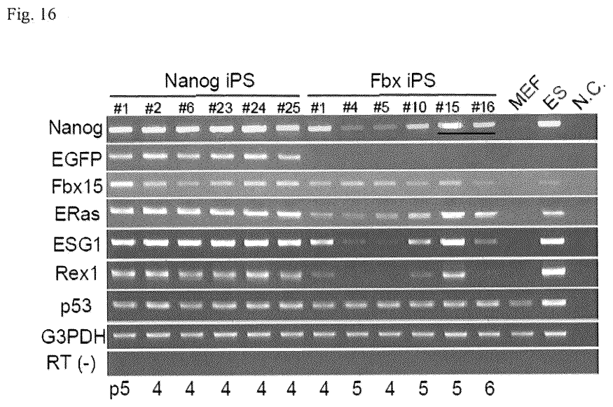

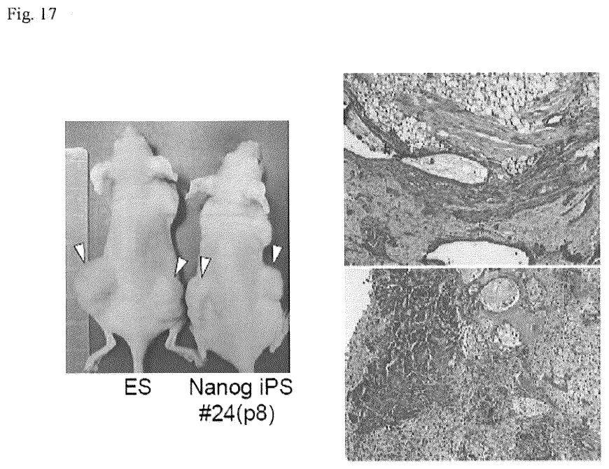

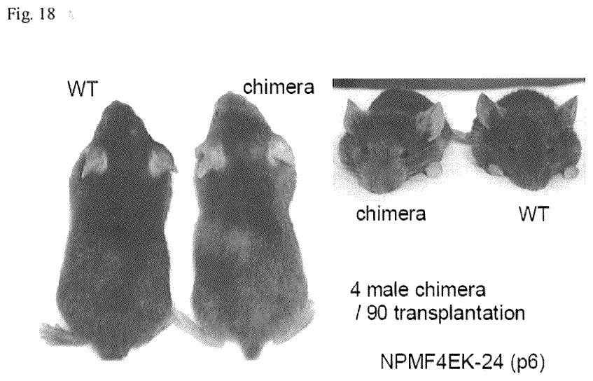

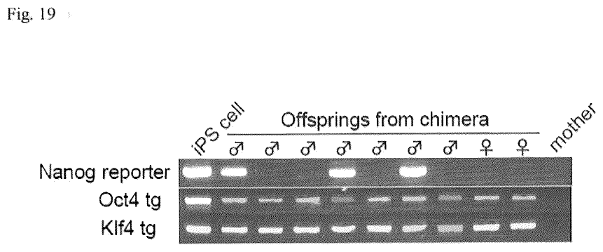

Studies were made as to whether iPS cells could be established with a reporter other than Fbx15-βgeo. Escherichia. coli artificial chromosome (BAC) containing the Nanog gene in the center was isolated, and then the GFP gene and the puromycin resistance gene were knocked in by recombination in E. coli (FIG. 12A). Subsequently, the above modified BAC was introduced into ES cells to confirm that the cells became GFP-positive in an undifferentiated state specific manner (data not shown). Then, these ES cells were transplanted in mouse blastocysts to create transgenic mice via chimeric mice. In these mice, GFP-positive cells were specifically observed in inner cell masses of the blastocysts or gonads of embryos at 13.5 days after fertilization (FIG. 12B). The gonads were removed from the embryos at 13.5 days after fertilization (hybrid of OBA, 129, and C57BL/6 mice), and MEFs were isolated. The isolated MEFs were confirmed to be GFP-negative (FIG. 13) by flow cytometry. These MEFs were retrovirally transduced with the 4 factors and subjected to puromycin selection, and as a result, a plural, number of resistant colonies were obtained. Only about 10 to 20% of the colonies were GFP-positive. When the GFP-positive colonies were passaged, they gave morphology (FIG. 14) and proliferation (FIG. 15) similar to those of ES cells. Examination of the gene expression pattern revealed that the expression pattern was closer to that of ES cells as compared to the iPS cells isolated from Fbx15^{βgeo/βgeo} MEFs by G418 selection (FIG. 16). When these cells were transplanted to nude mice, teratoma formation was induced, thereby the cells were confirmed to be iPS cells (FIG. 17). Further, chimeric mice were born by transplanting the iPS cells obtained by Nanog-GFP selection to the blastocysts of C57BL/6 mice (FIG. 18). When these chimeric mice were mated, germ-line transmission was observed (FIG. 19). In these iPS cells established by Nanog-GFP selection, which were closer to ES cells, the expressions of the 4 factors from the retroviruses were almost completely silenced, suggesting that self-replication was maintained by endogenous Oct3/4 and Sox2.

### Example 8: In Vitro Differentiation Induction

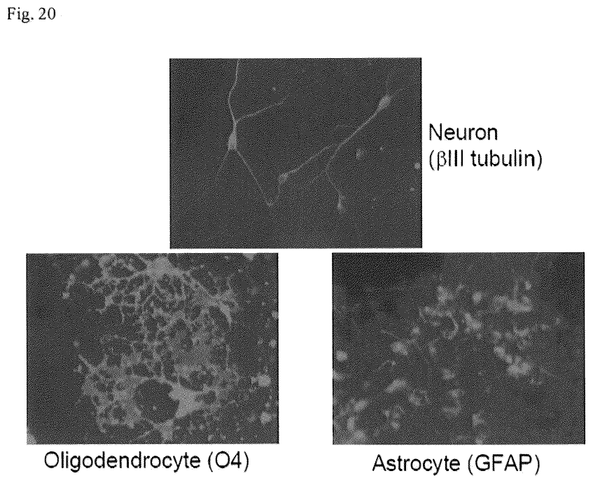

Confluent iPS cells in 10 cm dishes were trypsinized and suspended in ES cell medium (the STO cells were removed by adhesion to a gelatin-coated dish for 10 to 20 minutes after the suspension). 2×10⁶ cells were cultured for four days in a HEMA (2-hydroxyethyl methacrylate) coated E. coli culture dish as a suspension culture to form embryoid bodies (EBs) (day 1 to 4). On the 4^{th} day of EB formation (day 4), all of the EBs were transferred to a 10-cm tissue culture dish, and cultured in ES cell medium for 24 hours to allow adhesion. After 24 hours (day 5), the medium was changed to an ITS/fibronectin-containing medium. The culture was performed for 7 days (medium was exchanged every 2 days), and nestin-positive cells were selected (cells of other pedigrees were dying to some extent in a culture under serum-free condition) (day 5 to 12). A2B5-positive cells were then induced. After 7 days (day 12), the cells were separated by trypsinization, and the remaining EBs were removed. 1×10⁵ cells were seeded on a poly-L-omithine/fibronectin-coated 24-well plate, and cultured for 4 days in an N2/bFGF-containing medium (medium was exchanged every 2 days)(day p to 16). After 4 days (day 16), the medium was changed to an N2/bFGF/EGF-containing medium, and the culture was continued for 4 days (medium was exchanged every 2 days)(day 16 to 20). After 4 days (day 20), the medium was changed to an N2/bFGF/PDGF-containing medium, and the culture was continued for 4 days (medium was exchanged every 2 days)(day 20 to 24). During this period (day 12 to 24), when the cells had increased excessively and reached confluent, they were passaged at appropriate times, and 1 to 2×10⁵ cells were seeded (the number of the cells varied depending on the timing of the passage). After 4 days (day 24), the medium was changed to an N2/T3 medium, and the culture was continued for 7 days (day 24 to 31) with medium exchange every 2 days. On day 31, the cells were fixed and subjected to immunostaining. As a result, differentiation of the iPS cells into βIII tubulin-positive nerve cells, 04-positive oligodendrocytes, and GFAP-positive astrocytes was observed (FIG. 20).

Example 9: Establishment of iPS Cells without Drug Selection

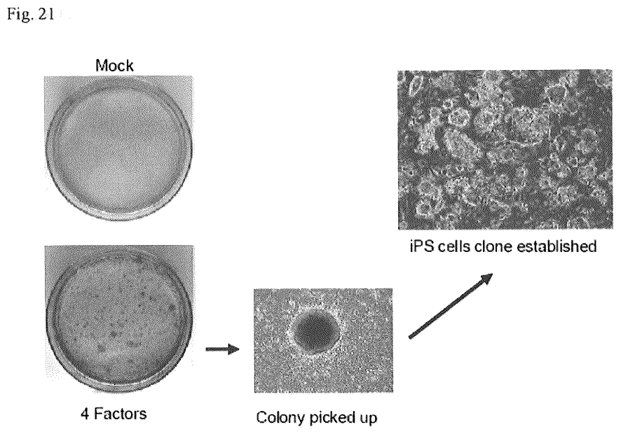

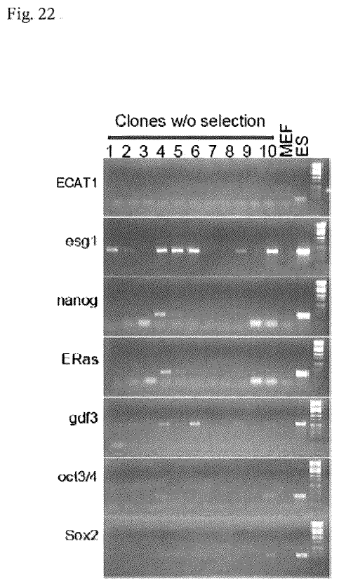

In order to establish iPS cells from arbitrary mouse somatic cells other than those derived from the Fbx15-βgeo knockin mouse, a method for the establishment without using drug selection was developed. 10,000, 50,000, or 100,000 cells mouse embryo fibroblasts (MEFs) were cultured on a 10 cm dish (on STO feeder cells). This is less than the number of cells used above, Control DNA or the 4 factors were retrovirally transduced. When culture was performed for 2 weeks in the ES cell medium (without G418 selection), no colony formation was observed in the dish in which the control DNA was transduced, whilst in the dish in which the 4 factors were transduced, a plurality of compact colonies were formed as well as flat colonies considered to be transformed (FIG. 21). When 24 colonies were picked up from these colonies and culture was continued, ES cell-like morphology was observed. Gene expression profiles thereof were examined by RT-PCR, and as a result, the expression of Esg1, an ES cell marker, was observed in 7 clones. Induction of many ES cell markers such as Nanog, ERas, GDF3, Oct3/4, and Sox2 was observed in clone 4, and therefore the cells were considered to be iPs cells (FIG. 22). The above results demonstrated that drug selection using Fbx15-βgeo knockin or the like was not indispensable for iPS cell establishment, and iPS cells could be established from arbitrary mouse-derived somatic cells. This also suggested the possibility that iPS cells could be established from somatic cells of a disease model mouse by the aforementioned technique.

Example 10: IPS Cell Generation from Hepatocytes and Gastric Mucous Cells

As cells from which iPS cells were induced, hepatocytes and gastric mucous cells being cells other than fibroblasts were examined. Hepatocytes were isolated from the liver of the Fbx15^{βgeo/βgeo} mice by perfusion. These hepatocytes were retrovirally introduced with the 4 factors, and then subjected to G418 selection to obtain plural iPS cell colonies. As a result of gene expression pattern analysis using a DNA microarray, the iPS cells derived from the liver were found to be more similar to ES cells than the iPS cells derived from dermal fibroblasts or embryonic fibroblasts. iPS cells were obtained also from gastric mucous cells in the same manner as those from hepatocytes.

### Example 11: Effect of MAP Kinase Inhibitor on iPS Cell Establishment

PD98059 is an inhibitor of MAP kinase which suppresses proliferation of various differentiated cells. However, it is known to promote maintenance of undifferentiated status and proliferation of ES cells. Effects of PD98059 on iPS cell establishment were thus examined. MEFs established from a mouse having the selective markers of Nanog-EGFP-IRES-Puro were retrovirally introduced with the 4 factors and subjected to puromycin selection. When PD98059 was not added, the percentage of GFP-positive colonies was 8% of the iPS cell colonies obtained. However, in the group to which PD98059 (final concentration: 25 μM) was continuously added from the next day of the retroviral transfection, 45% of the colonies obtained were GFP-positive. The results were interpreted to be due to PD98059 promoting the proliferation of the GFP-positive iPS cells, which are closer to ES cells, whilst PD98059 suppressing the proliferation of the GFP-negative iPS cells or differentiated cells. From these results, PD98059 was demonstrated to be able to be used for establishment of the iPS cells closer. to ES cells or establishment of iPS cells without using drug selection.

Example 12: Establishment of iPS Cells from Embryonic HDFs in Mouse ES Cell Medium

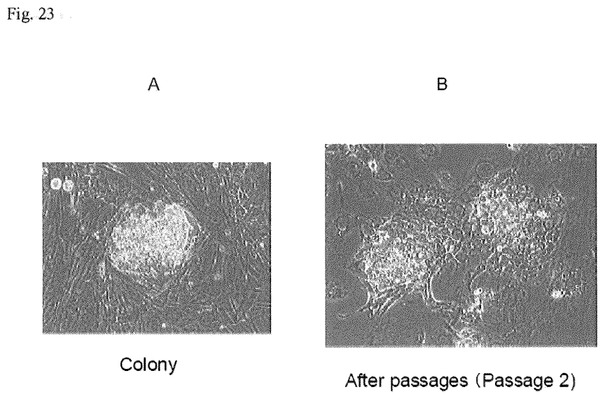

A plasmid, containing the red fluorescence protein gene downstream from the mouse Oct3/4 gene promoter and the hygromycin resistance gene downstream from the PGK promoter, was introduced by nucleofection into embryonic human dermal fibroblasts (HDFs) in which solute carrier family 7 (Slc7a1, NCBI accession number NM_007513) as a mouse ecotropic virus receptor was expressed by lentiviral transduction. Hygromycin selection was performed to establish strains with stable expression. 800,000 cells were seeded on the STO cells treated with mitomycin, and on the next day, Oct3/4, Sox2, Klf4, and c-Myc (each derived from human) were retrovirally transduced into the cells. 24 colonies were picked up from those obtained after 3 weeks (FIG. 23, left), and transferred on a 24-well plate on which the STO cells were seeded and then cultured. After 2 weeks, one grown clone was passaged on a 6-well plate on which the STO cells were seeded and cultured. As a result, cells morphologically similar to ES cells were obtained (FIG. 23, right), suggesting that the cells were iPS cells. The mouse ES cell medium was used as every medium.

Example 13: Establishment of iPS Cells from Adult HDFs in Mouse ES Cell Medium

Human adult dermal fibroblasts (adult HDFs) or human neonatal foreskin cells (BJ) were transduced with Slc7a1 (mouse retroviral receptor) by using lentivirus, and the resulting cells were seeded on 800,000 feeder cells (mitomycin-treated STO cells). The genes were retrovirally transduced as the following combinations.
1. Oct3/4, Sox2, Klf4, c-Myc, TERT, and SV40 Large T antigen
2. Oct3/4, Sox2, Klf4, c-Myc, TERT, HPV16 E6
3. Oct3/4, Sox2, Klf4, c-Myc, TERT, HPV16 E7
4. Oct3/4, Sox2, Klf4, c-Myc, TERT, HPV16 E6, HPV16 E7
5. Oct3/4, Sox2, Klf4, c-Myc, TERT, Bmi1
6. Oct3/4, Sox2, Klf4, c-Myc
7. Oct3/4, Sox2, Klf4, c-Myc and TERT were derived from human, and Bmi1 was derived from mouse)

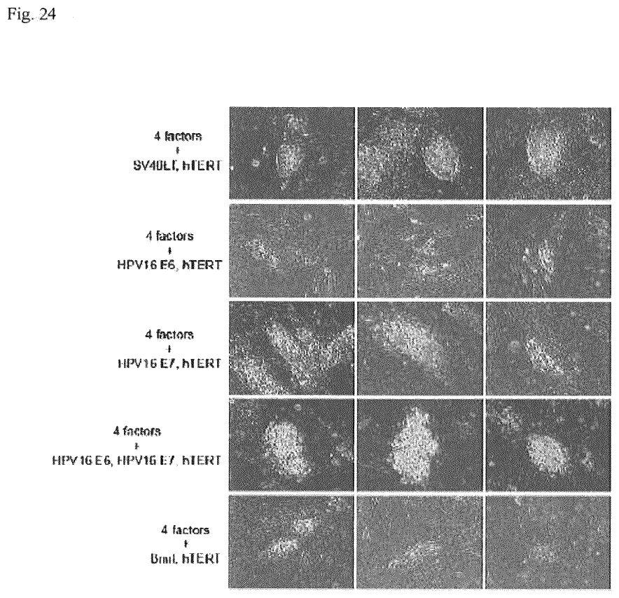

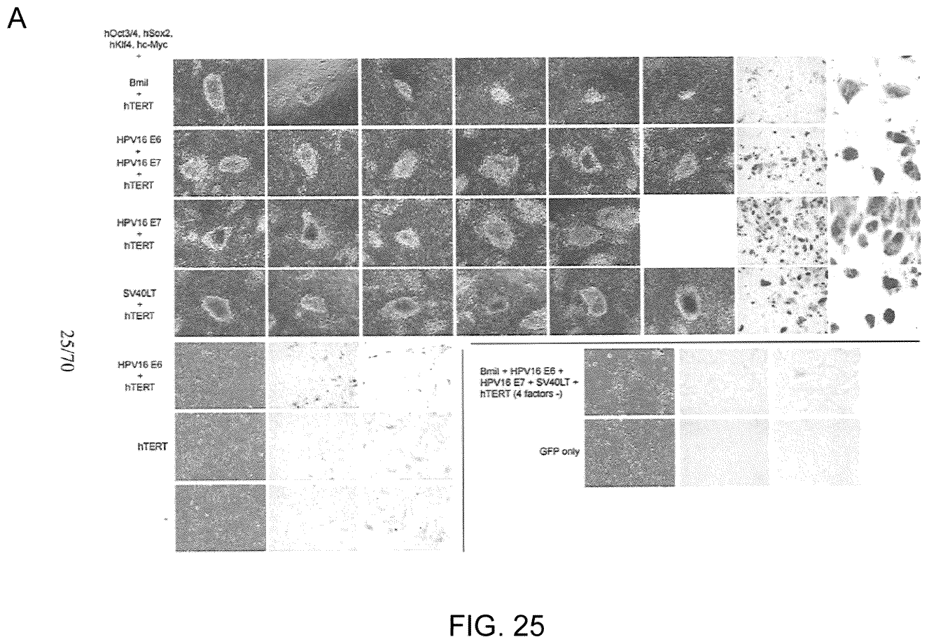

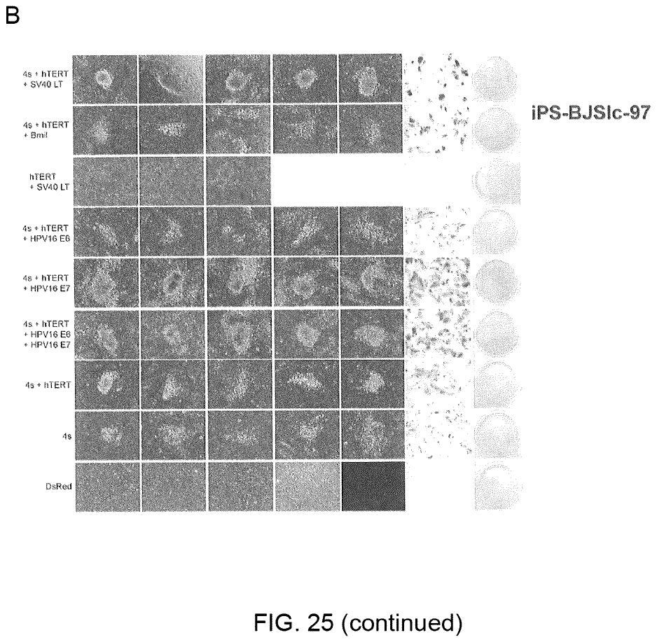

The culture was continued under the culture conditions for mouse ES cells without drug selection. As a result, colonies considered to be those of iPS cells emerged on the 8^{th} day after the virus transfection on the dish in which the factors were introduced according to Combination 1 (FIG. 24). iPS cell-like colonies also emerged with the other combinations (2 to 5), although they were not as apparent when compared to Combination 1. When only the 4 factors were transduced (6), no colonies emerged. However, cells transduced with the four factors under the experimental conditions used in this Example did show staining for alkaline phosphatase (FIG. 25(A)-(B)).

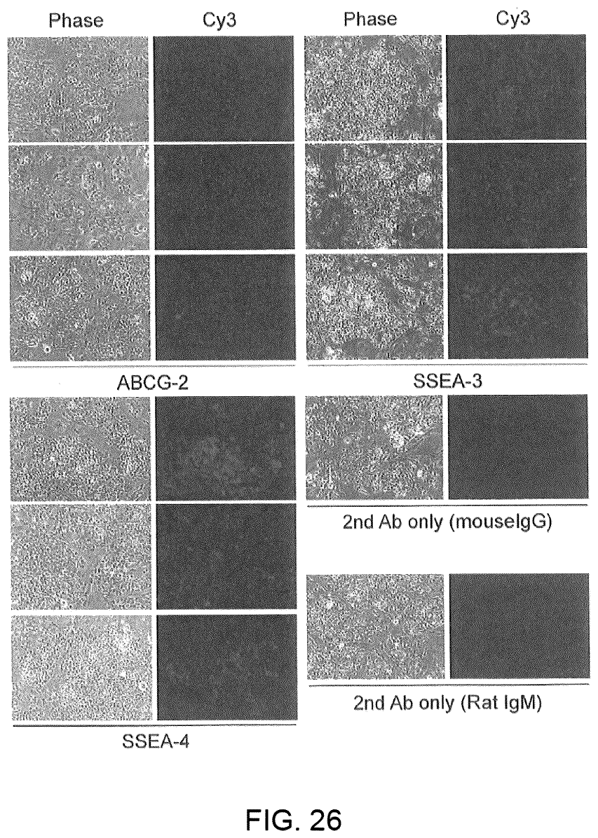

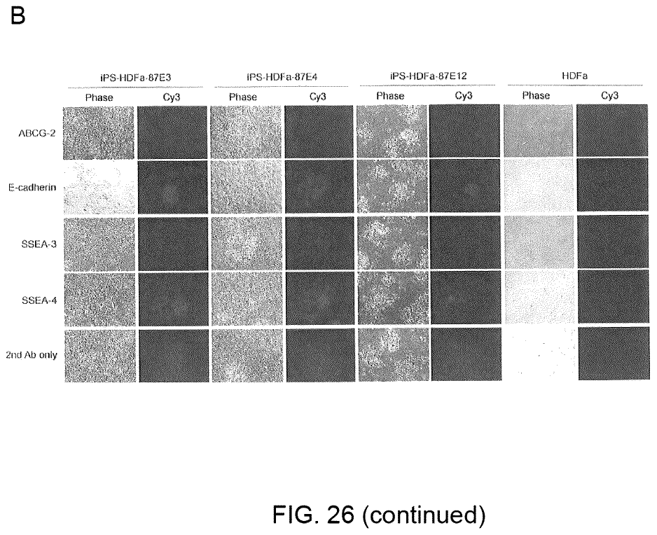

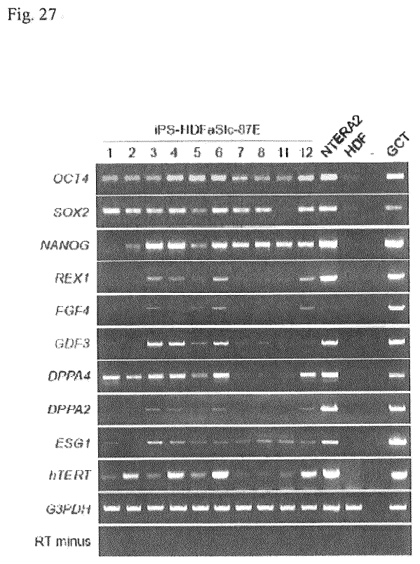

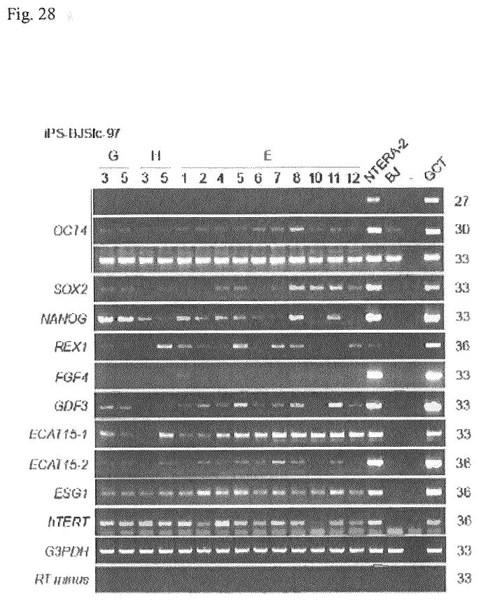

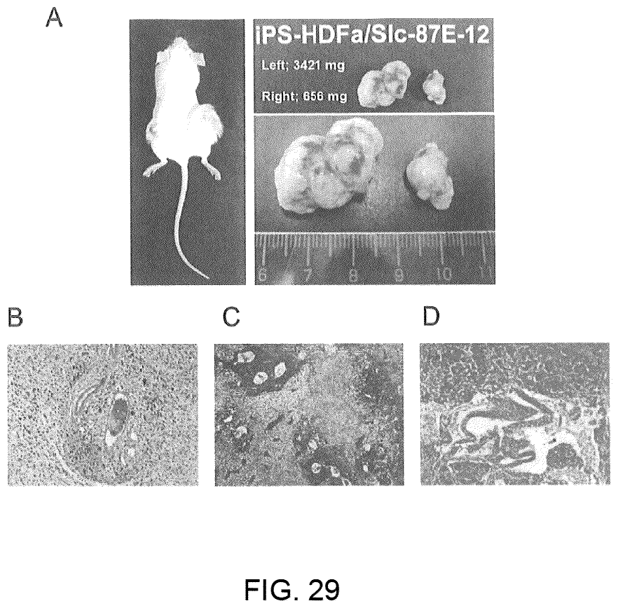

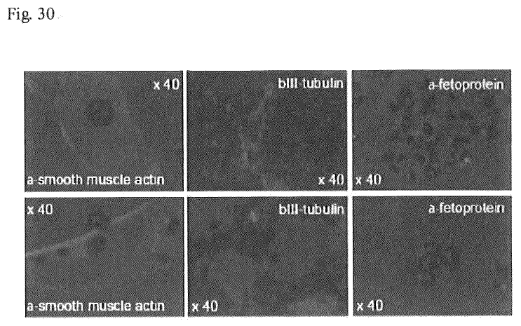

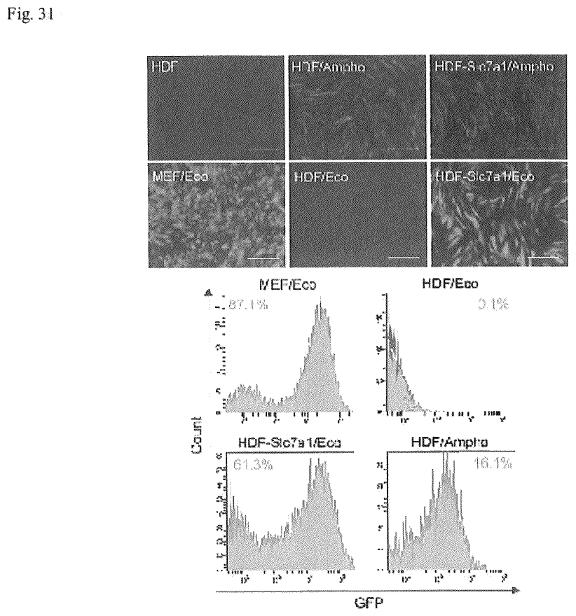

Optimization of the methods heretofore described revealed successful induction of iPS cell colonies through staining of any number of pluripotent markers, including alkaline phosphatase, ABCG-2, E-cadherin, SSEA-3, and SSEA-4 when adult human dermal fibroblasts expressing mouse Slc7a1 gene were generated into iPS cells by reprogramming with the four factors plus TERT and SV40 Large T antigen (i.e. six factors total: c-Myc, Klf4, Sox2, Oct3/4, TERT, and SV40 Large T antigen) (FIG. 26(A)-(B)). These cells were assessed for pluripotent cell markers (FIG. 27). These cells were found to express ECATS, including Nanog and ESG1. Similarly, BJ fibroblasts expressing mouse Slc7a1 gene were generated into iPS cells by reprogramming with the following same factors. These cells were also tested for pluripotent cell markers (FIG. 28). In addition, iPS cells generated from adult HDFs were selected for subcutaneous injection into the dorsal flanks of SCID mice. Teratoma formation was observed (FIGS. 29(A)-(D)). Human dermal fibroblasts were also shown to differentiate in vitro upon culturing in HEMA-coated plates (7 days) and gelatinized dishes (7 days) (FIG. 30).

### Example 14: Optimization of Retroviral Transduction for Generating Human iPS Cells

Next, iPS cell generation from adult human somatic cells was further evaluated by optimizing retroviral transduction in human fibroblasts and subsequent culture conditions. Induction of iPS cells from mouse fibroblasts requires retroviruses with high transduction efficiencies (Takahashi et al. Cell 126:663-676, 2006). Therefore, transduction methods in adult human dermal fibroblasts (HDFs) were optimized. First, green fluorescent protein (GFP) was introduced into adult HDF with amphotropic retrovirus produced in PLAT-A packaging cells. As a control, GFP was introduced into mouse embryonic fibroblasts (MEF) with ecotropic retrovirus produced in PLAT-E packaging cells (Morita et al. Gene Ther. 7:1063-66, 2000). In MEF, more than 80% of cells expressed GFP (FIG. 31). In contrast, less than 20% of HDF expressed GFP with significantly lower intensity than in MEF. To improve the transduction efficiency, the mouse receptor for retroviruses, Slc7a1 (Verrey et al. Pflugers Arch. 447:532-542, 2004) (also known as mCAT1), was introduced into HDF with lentivirus. Then GFP was introduced into HDF-Slc7a1 with ecotropic retrovirus. This strategy yielded a transduction efficiency of 60%, with a similar intensity to that in MEF.

Example 15: Generation of iPS Cells from Adult HDFs in Primate ES Cell Culture Medium

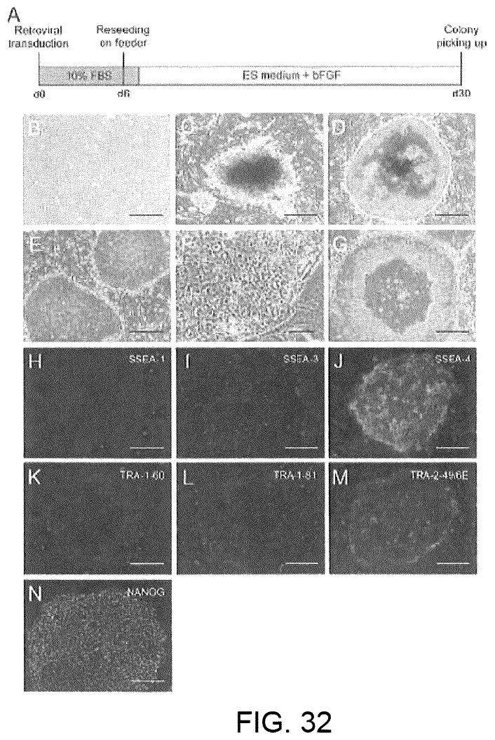

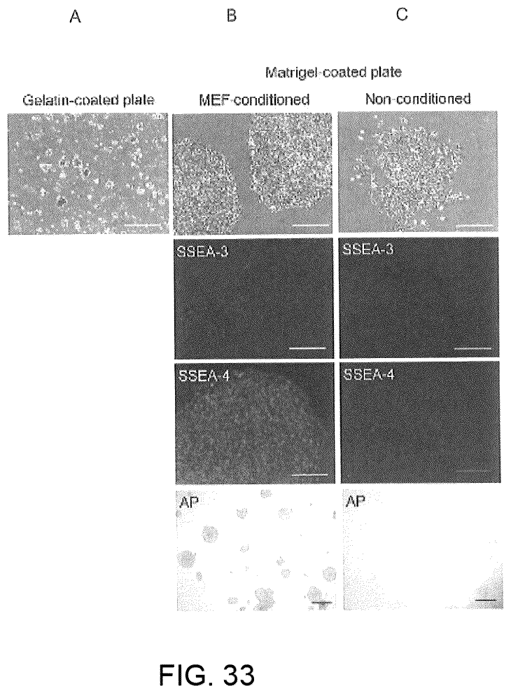

The protocol for human iPS cell induction is summarized in FIG. 32A. pMXs encoding human Oct3/4, Sox2, Klf4, and c-Myc were introduced into HDF-Slc7a1 cells (FIG. 32B; 8×10⁵ cells per 100 mm dish). The HDFs were derived from facial dermis of 36-year-old Caucasian female.

Six days after transduction, the cells were harvested by trypsinization and plated onto mitomycin C-treated SNL feeder cells (McMahon et al. Cell 62:1073-85, 1990) at 5×10⁴ or 5×10⁵ cells per 100 mm dish. The next day, the medium (DMEM containing 10% FBS) was replaced with a medium for primate ES cell culture supplemented with 4 ng/ml basic fibroblast growth factor (bFGF). Approximately two weeks later, some granulated colonies appeared that were not similar to hES cells in morphology (FIG. 32C). Around day 25, distinct types of colonies that were flat and resembled hES cell colonies were observed (FIG. 32D). From 5×10⁴ fibroblasts, ~10 hES cell-like colonies and ~100 granulated colonies (7/122, 8/84, 8/171, 5/73, 6/122, and 11/213 in six independent experiments, summarized in TABLE 8) were observed.

TABLE 8
Summary of the iPS cell induction experiments

| | | Cell No. | No. of | No. of | No. of | No. of |
|---|---|---|---|---|---|---|
| | Parental | seeded at | ES-like | total | picked up | established |
| Exp. ID | cells | d6 | colony | colony | colony | clone |
| 201B | HDF | 50000 | 7 | 129 | 7 | 5 |
| 243H | HFLS | 500000 | 0 | >1000 | | |
| | | 50000 | 17 | 679 | 6 | 2 |
| 246B | HDF | 500000 | 0 | 420 | | |
| | | 500000 | 2 | 508 | | |
| | | 50000 | 8 | 92 | 6 | 6 |
| 246G | BJ | 50000 | 7 | 10 | 6 | 5 |
| | | 500000 | 86 | 98 | | |
| | | 500000 | 106 | 108 | | |
| 249D | HDF | 500000 | 0 | 320 | | |
| | | 500000 | 0 | 467 | | |
| | | 50000 | 8 | 179 | 6 | 4 |
| 253F | HDF | 50000 | 5 | 78 | 3 | 2 |
| | | 50000 | 6 | 128 | 3 | 3 |
| | | 500000 | 10 | 531 | | |
| | | 500000 | 3 | 738 | | |
| 282C | HDF | 50000 | 11 | 224 | 3 | 1 |
| 282H | BJ | 50000 | 13 | 15 | 3 | 2 |
| 282R | HFLS | 5000 | 31 | 98 | 6 | 2 |

At day 30, hES cell-like colonies were picked up and mechanically disaggregated into small clumps without enzymatic digestion. When starting with 5×10⁵ fibroblasts, the dish was nearly covered with more than 300 granulated colonies. Occasionally some hES cell-like colonies in between the granulated cells were observed, but it was difficult to isolate hES cell-like colonies because of the high density of granulated cells.

The hES-like cells expanded on SNL feeder cells under the human ES cell culture condition. They formed tightly packed and flat colonies (FIG. 32E). Each cell exhibited morphology similar to that of human ES cells, characterized by large nucleoli and scant cytoplasm (FIG. 32F). As is the case with hES cells, occasionally spontaneous differentiation was observed in the center of the colony (FIG. 320).

These cells also showed similarity to hES cells in feeder dependency. They did not attach to gelatin-coated tissue-culture plates. By contrast, they maintained an undifferentiated state on Matrigel-coated plates in MEF-conditioned medium (MEF-CM), but not in ES medium (FIG. 33).

Since these cells were indistinguishable from hES cells in morphology and other aspects noted above, the selected cells after transduction of HDFs are referred to as human iPS cells. The molecular and functional evidence for this claim is further described below. Human iPS cells clones established in this study are summarized in TABLE 9.

TABLE 9
Characterization of established clones

| | | Marker | | Pluripotency | | | |
|---|---|---|---|---|---|---|---|
| | | expression | | | | Cardio- | |
| Clone | Source | RT-PCR | IC | EB | PA6 | myocyte | Teratoma |
| 201B1 | HDF | √ | | | | | |
| 201B2 | | √ | √ | √ | √ | √ | |
| 201B3 | | √ | | | | | |
| 201B6 | | √ | √ | √ | √ | √ | |
| 201B7 | | √ | √ | √ | √ | √ | √ |
| 243H1 | HFLS | √ | | √ | | | |
| 243H7 | | √ | | √ | | | |
| 246B1 | HDF | √ | | | | | |
| 246B2 | | √ | | | | | |
| 246B3 | | √ | | | | | |
| 246B4 | | √ | | | | | |
| 246B5 | | √ | | | | | |
| 246B6 | | √ | | | | | |
| 246G1 | BJ | √ | | √ | | | |
| 246G3 | | √ | | √ | | | |
| 246G4 | | √ | | | | | |
| 246G5 | | √ | | | | | |
| 246G6 | | √ | | | | | |
| 253F1 | HDF | √ | | | | | |
| 253F2 | | √ | | | | | |
| 253F3 | | √ | | | | | |
| 253F4 | | √ | | | | | |
| 253F5 | | √ | | | | | |

IC; immunocytochemistry,
EB; embryoid body

### Human iPS Cells Express hES Markers

In general, except for a few cells at the edge of the colonies, human iPS cells did not express stage-specific embryonic antigen (SSEA)-1 (FIG. 32H). In contrast, they expressed hES cell-specific surface antigens (Adewumi et al. Nat. Biotechnol. 25:803-816, 2007), including SSEA-3, SSEA-4, tumor-related antigen (TRA)-1-60, TRA-1-81, and TRA-2-49/6E (alkaline phosphatase), and NANOG protein (FIG. 321-N).

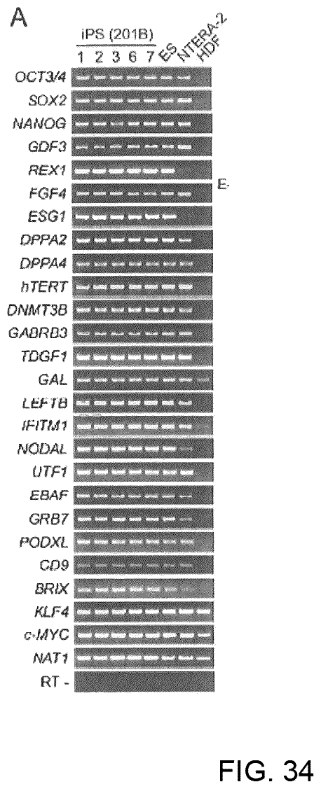

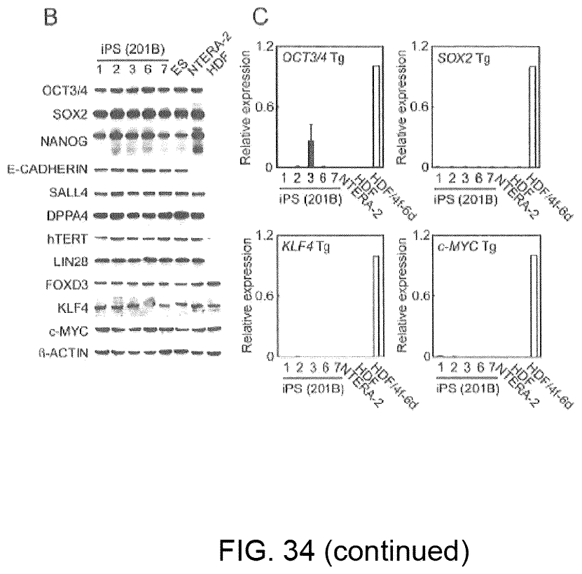

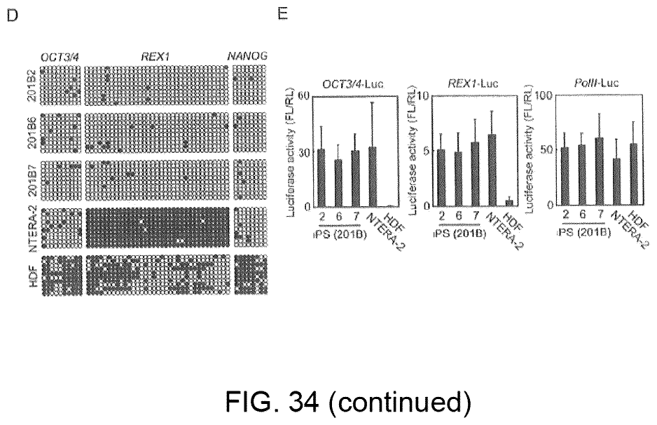

RT-PCR showed human iPS cells expressed many undifferentiated ES cell gene markers (Adewumi et al. Nat. Biotechnol. 25:803-816, 2007), such as OCT3/4, SOX2, NANOG, growth and differentiation factor 3 (GDF3), reduced expression 1 (REX1), fibroblast growth factor 4 (FGF4), embryonic cell-specific gene 1 (ESG1), developmental pluripotency-associated 2 (DPPA2), DPPA4, and telomerase reverse transcriptase (hTER1) at levels equivalent to or higher than those in the human embryonic carcinoma cell line, NTERA-2 (FIG. 34A). By western blotting, proteins levels of OCT3/4, SOX2, NANOG, SALL4, E-CADHERIN, and hTERT were similar in human iPS cells and hES cells (FIG. 34B). In human iPS cells, the expression of transgenes from integrated retroviruses was efficiently silenced, indicating that they depend on the endogenous expression of these genes (FIG. 34C).

### Promoters of ES Cell-Specific Genes are Active in Human iPS Cells

Bisulfite genomic sequencing analyses evaluating the methylation statuses of cytosine guanine dinucleotides (CpG) in the promoter regions of pluripotent-associated genes, such as OCT3/4, REX1, and NANOG, revealed that they were highly unmethylated, whereas the CpG dinucleotides of the regions were highly methylated in parental HDFs (FIG. 34D). These findings indicate that these promoters are active in human iPS cells.

Luciferase reporter assays also showed that human OCT3/4 and REX1 promoters had high levels of transcriptional activity in human iPS cells, but not in HDF. The promoter activities of ubiquitously expressed genes, such as human RNA polymerase II (PolII), showed similar activities in both human iPS cells and HDF (FIG. 34E).

### High Telomerase Activity and Exponential Growth of Human iPS Cells

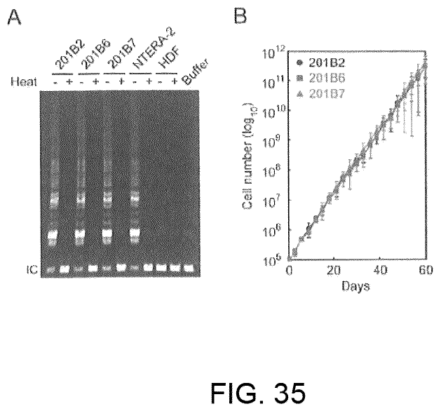

As predicted from the high expression levels of hTERT, human iPS cells showed high telomerase activity (FIG. 35A). They proliferated exponentially for at least 4 months (FIG. 35B). The calculated doubling time of human iPS cells were 46.9±12.4 (clone 201B2), 47.8±6.6 (201B6) and 43.2±11.5 (201B7) hours (FIG. 35B). These times are equivalent to the reported doubling time of hES cells (Cowan et al. N Engl. J Med. 350:1353-56, 2004).

Human iPS Cells are Derived from HDF, not Cross-Contamination

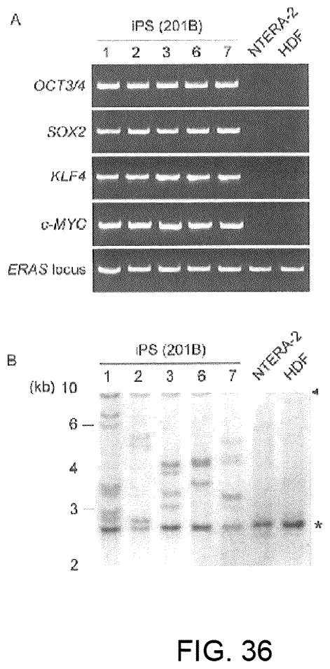

PCR of genomic DNA of human iPS cells showed that all clones have integration of all the four retroviruses (FIG. 36A). Southern blot analysis with a c-Myc cDNA probe revealed that each clone had a unique pattern of retroviral integration sites (FIG. 36B). In addition, the patterns of 16 short tandem repeats were completely matched between human iPS clones and parental HDF (TABLE 10).

TABLE 10
STR analyses of HDF-derived iPS cells

| Locus/ | | | | | | | | | | | | | | |
|---|---|---|---|---|---|---|---|---|---|---|---|---|---|---|
| Clone | 201B1 | | 201B2 | | 201B3 | | 201B6 | | 201B7 | | NTERA-2 | | HDF | |
| D3S1358 | 15 | 17 | 15 | 17 | 15 | 17 | 15 | 17 | 15 | 17 | 15 | | 15 | 17 |
| TH01 | 5 | | 5 | | 5 | | 5 | | 5 | | 9 | | 5 | |
| D21S11 | 28 | | 28 | | 28 | | 28 | | 28 | | 29 | 30 | 28 | |
| D18S51 | 14 | | 14 | | 14 | | 14 | | 14 | | 13 | | 14 | |
| Penta_E | 7 | 19 | 7 | 19 | 7 | 19 | 7 | 19 | 7 | 19 | 5 | 14 | 7 | 19 |
| D5S818 | 11 | | 11 | | 11 | | 11 | | 11 | | 8 | 11 | 11 | |
| D13S317 | 10 | 14 | 10 | 14 | 10 | 14 | 10 | 14 | 10 | 14 | 14 | | 10 | 14 |
| D7S820 | 9 | 10 | 9 | 10 | 9 | 10 | 9 | 10 | 9 | 10 | 12 | | 9 | 10 |
| D16S539 | 11 | 13 | 11 | 13 | 11 | 13 | 11 | 13 | 11 | 13 | 11 | 16 | 11 | 13 |
| CSF1PO | 10 | | 10 | | 10 | | 10 | | 10 | | 9 | 11 | 10 | |
| Penta_D | 8 | 10 | 8 | 10 | 8 | 10 | 8 | 10 | 8 | 10 | 11 | 12 | 8 | 10 |
| AMEL | X | | X | | X | | X | | X | | X | Y | X | |
| vWA | 15 | 18 | 15 | 18 | 15 | 18 | 15 | 18 | 15 | 18 | 19 | | 15 | 18 |
| D8S1179 | 8 | 10 | 8 | 10 | 8 | 10 | 8 | 10 | 8 | 10 | 13 | 15 | 8 | 10 |
| TPOX | 8 | 9 | 8 | 9 | 8 | 9 | 8 | 9 | 8 | 9 | 8 | 9 | 8 | 9 |
| FGA | 20 | 22 | 20 | 22 | 20 | 22 | 20 | 22 | 20 | 22 | 23 | | 20 | 22 |

These patterns differed from any established hES cell lines reported on National Institutes of Health website: (stemcells.nih.gov/research/nihresearch/scunit/genotyping). In addition, chromosomal G-band analyses showed that human iPS cells had a normal karyotype of 46XX (not shown). Thus, human iPS clones were derived from HDF and were not a result of cross-contamination.

### Example 16: Embryoid Body-Mediated Differentiation of Human iPS Cells

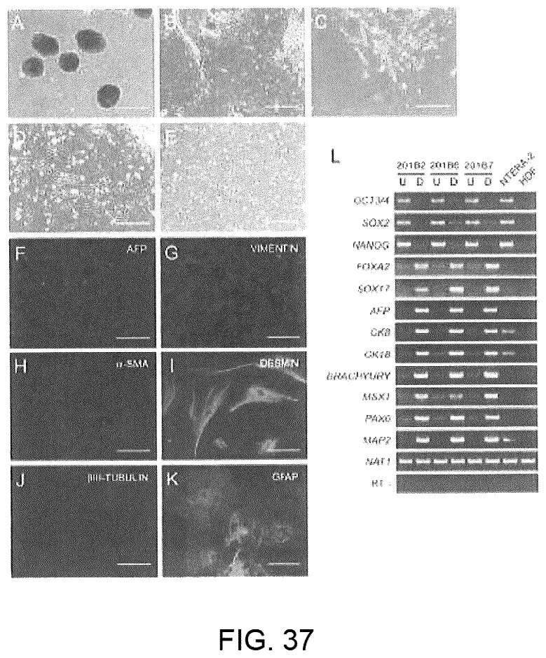

To determine the differentiation ability of human iPS cells in vitro, floating cultivation was used to form embryoid bodies (EBs) (Itskovitz-Eldor et al. Mol. Med. 6:88-95, 2000). After 8 days in suspension culture, iPS cells formed ball-shaped structures (FIG. 37A). These embryoid body-like structures were transferred to gelatin-coated plates and continued cultivation for another 8 days. Attached cells showed various types of morphologies, such as those resembling neuronal cells, cobblestone-like cells, and epithelial cells (FIG. 37B-E). Immunocytochemistry detected cells positive for βIII-tubulin (a marker of ectoderm), glial fibrillary acidic protein (GFAP, ectoderm), a-smooth muscle actin (α-SMA, mesoderm), desmin (mesoderm), α-fetoprotein (AFP, endoderm), and vimentin (mesoderm and parietal endoderm) (FIG. 37F-K). RT-PCR confirmed that these differentiated cells expressed forkhead box A2 (FOXA2, a marker of endoderm), AFP (endoderm), cytokeratin 8 and 18 (endoderm), SRY-box containing gene 17 (SOX17, endoderm), BRACHYURY (mesoderm), Msh homeobox 1 (MSX1, mesoderm), microtubule-associated protein 2 (MAP2, ectoderm), and paired box 6 (PAX6, ectoderm) (FIG. 37L). In contrast, expression of OCT3/4, SOX2, and NANOG was significantly decreased. These data demonstrated that iPS cells could differentiate into three germ layers in vitro.

Example 17: Directed Differentiation of Human iPS Cells into Neural Cells

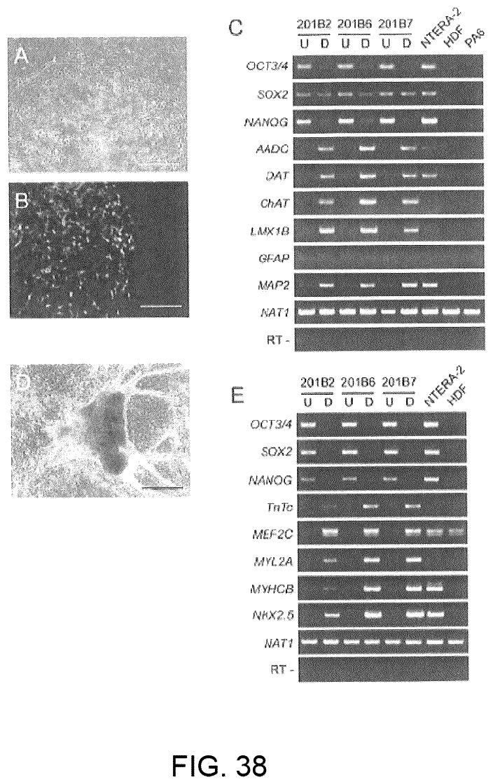

Next, it was examined whether lineage-directed differentiation of human iPS cells could be induced by reported methods for hES cells. Human iPS cells were seeded on PA6 feeder layer and maintained under differentiation conditions for 2 weeks (Kawasaki et al. Neuron 28:31-40, 2000). Cells spread drastically, and some neuronal structures were observed (FIG. 38A). Immunocytochemistry detected cells positive for, tyrosine hydroxylase and βIII tubulin in the culture (FIG. 38B). PCR analysis revealed expression of dopaminergic neuron markers, such as aromatic-L-amino acid decarboxylase (AADC), choline acetyltransferase (ChAT), solute carrier family 6 (neurotransmitter transporter, dopamine), member 3 (DAD, and LIM homeobox transcription factor 1 beta (LMX1B), as well as another neuron marker, MAP2 (FIG. 38C). In contrast, GFAP expression was not induced with this system. On the other hand, expression of OCT3/4, SOX2, and NANOG decreased (FIG. 38C). These data demonstrated that iPS cells could differentiate into neuronal cells, including dopaminergic neurons, by co-culture with PA6 cells.

Example 18: Directed Differentiation of Human iPS Cells into Cardiac Cells

Next directed cardiac differentiation of human iPS cells was examined with the recently reported protocol, which utilizes activin A and bone morphogenetic protein (BMP) 4 (Laflamme et al. Nat. Biotechnol. 25:1015-24, 2007). Twelve days after the induction of differentiation, clumps of cells started beating (FIG. 38D). RT-PCR showed that these cells expressed cardiomyocyte markers, such as troponin T type 2 cardiac (TnTc); myocyte enhancer factor 2C (MEF2C); NK2 transcription factor related, locus 5 (NKX2.5) myosin, light polypeptide 7, regulatory (MYL2A), and myosin, heavy polypeptide 7, cardiac muscle, beta (MYHCB) (FIG. 38E). In contrast, the expression of Oct3/4, Sox2, and Nanog markedly decreased. Thus, human iPS cells can differentiate into cardiac myocytes in vitro.

Example 19: Teratoma Formation from Human iPS Cells

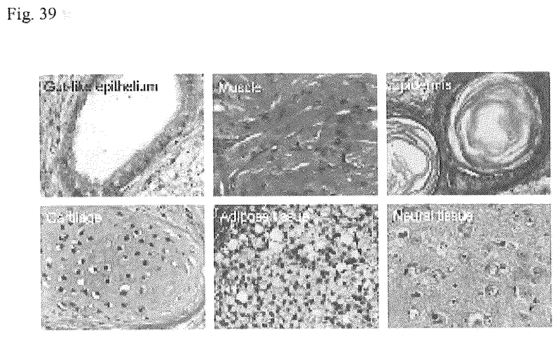

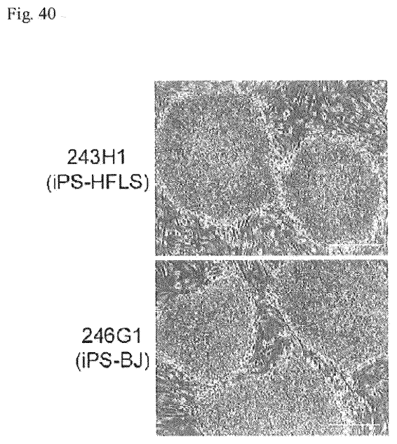

To test pluripotency in vivo, human iPS cells (clone 20187) were transplanted subcutaneously into dorsal flanks of immunodeficient (SCID) mice. Nine weeks after injection, tumor formation was observed. Histological examination showed that the tumor contained various tissues (FIG. 39), including gut-like epithelial tissues (endoderm), striated muscle (mesoderm), cartilage (muscle), neural tissues (ectoderm), and keratin-containing squamous tissues (ectoderm).

Example 20: Generation of iPS Cells from Other Human Somatic Cells

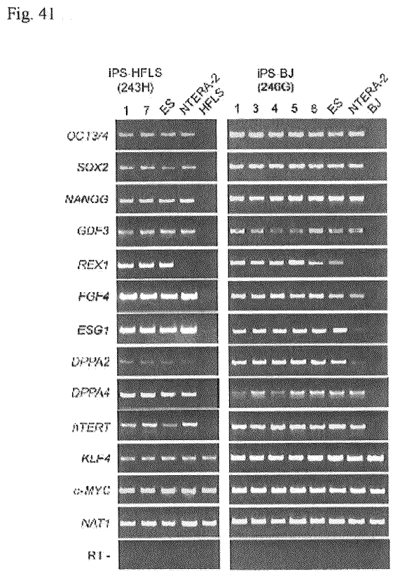

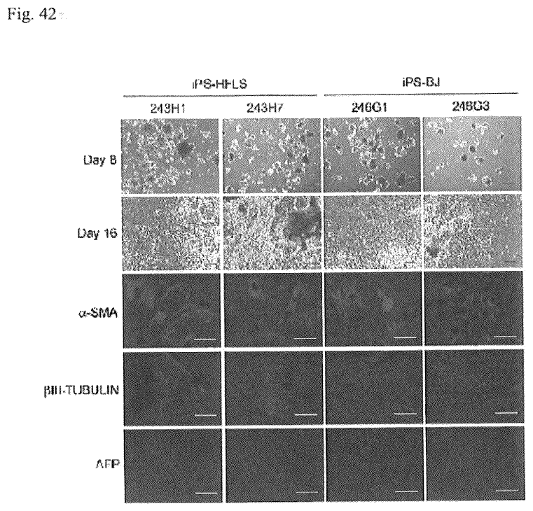

In addition to HDF, primary human fibroblast-like synoviocytes (HFLS) from synovial tissue of 69-year-old Caucasian male and BJ cells, a cell line established from neonate fibroblasts, were used (TABLE 8). From 5×10⁴ HFLS cells per 100 mm dish, more than 600 hundred granulated colonies and 17 hES cell-like colonies were obtained. Six colonies were picked, of which only two were expandable as iPS cells (FIG. 40). Dishes seeded with 5×10⁵ HFLS were covered with granulated cells, and no hES cell-like colonies were distinguishable. In contrast, 7 to 8 and ~100 hES cell-like colonies were obtained from 5×10⁴ and 5×10⁵ BJ cells, respectively, with only a few granulated colonies (TABLE 8). Six hES cell-like colonies were picked and iPS cells were generated from five colonies (FIG. 40). Human iPS cells derived from HFLS and BJ expressed hES cell-marker genes at levels similar to or higher than those in hES cells (FIG. 41). They differentiated into all three germ layers through EBs (FIG. 42). STR analyses confirmed that iPS-HFLS cells and iPS-BJ cells were derived from HFLS and BJ fibroblasts, respectively (TABLE 11 and TABLE 12).

TABLE 11
STR analyses of HFLS-derived iPS cells

| Locus/Clone | 243H1 | | 243H7 | | HFLS | |
|---|---|---|---|---|---|---|
| D3S1358 | 16 | 17 | 16 | 17 | 16 | 17 |
| TH01 | 5 | 9 | 5 | 9 | 5 | 9 |
| D21S11 | 28 | 30 | 28 | 30 | 28 | 30 |
| D18S51 | 14 | 17 | 14 | 17 | 14 | 17 |
| Penta_E | 5 | 12 | 5 | 12 | 5 | 12 |
| D5S818 | 10 | 12 | 10 | 12 | 10 | 12 |
| D13S317 | 13 | | 13 | | 13 | |
| D7S820 | 9 | 12 | 9 | 12 | 8 | 12 |
| D16S539 | 11 | 13 | 11 | 13 | 11 | 13 |
| CSF1PO | 10 | 11 | 10 | 11 | 10 | 11 |
| Penta_D | 9 | 11 | 9 | 11 | 9 | 11 |
| AMEL | X | | X | Y | X | Y |
| vWA | 17 | 19 | 17 | 19 | 17 | 19 |
| D8S1179 | 13 | | 13 | | 13 | |
| TPOX | 8 | 11 | 8 | 11 | 8 | 11 |
| FGA | 21 | 22 | 21 | 22 | 21 | 22 |

TABLE 12
STR analyses of BJ-derived iPS cells

| Locus/ | | | | | | | | | | | | |
|---|---|---|---|---|---|---|---|---|---|---|---|---|
| Clone | 246G1 | | 246G3 | | 246G4 | | 246G5 | | 246G6 | | BJ | |
| D3S1358 | 13 | 15 | 13 | 15 | 13 | 15 | 13 | 15 | 13 | 15 | 13 | 15 |
| TH01 | 6 | 7 | 6 | 7 | 6 | 7 | 6 | 7 | 6 | 7 | 6 | 7 |
| D21S11 | 28 | | 28 | | 28 | | 28 | | 28 | | 28 | |
| D18S51 | 16 | 18 | 16 | 18 | 16 | 18 | 16 | 18 | 16 | 18 | 16 | 18 |
| Penta_E | 7 | 17 | 7 | 17 | 7 | 17 | 7 | 17 | 7 | 17 | 7 | 17 |
| D5S818 | 11 | | 11 | | 11 | | 11 | | 11 | | 11 | |
| D13S317 | 9 | 10 | 9 | 10 | 9 | 10 | 9 | 10 | 9 | 10 | 9 | 10 |
| D7S820 | 11 | 12 | 11 | 12 | 11 | 12 | 11 | 12 | 11 | 12 | 11 | 12 |
| D16S539 | 9 | 13 | 9 | 13 | 9 | 13 | 9 | 13 | 9 | 13 | 9 | 13 |
| CSF1PO | 9 | 11 | 9 | 11 | 9 | 11 | 9 | 11 | 9 | 11 | 9 | 11 |
| Penta_D | 11 | 12 | 11 | 12 | 11 | 12 | 11 | 12 | 11 | 12 | 11 | 12 |
| AMEL | X | Y | X | Y | X | Y | X | Y | X | Y | X | Y |
| vWA | 16 | 18 | 16 | 18 | 16 | 18 | 16 | 18 | 16 | 18 | 16 | 18 |
| D8S1179 | 9 | 11 | 9 | 11 | 9 | 11 | 9 | 11 | 9 | 11 | 9 | 11 |
| TPOX | 10 | 11 | 10 | 11 | 10 | 11 | 10 | 11 | 10 | 11 | 10 | 11 |
| FGA | 22 | 23 | 22 | 23 | 22 | 23 | 22 | 23 | 22 | 23 | 22 | 23 |

Thus, with Examples 13-20 it was shown that iPS cells can be generated from adult HDF and other somatic cells by retroviral transduction of the same four transcription factors, namely Oct3/4, Sox2, Klf4, and c-Myc. The established human iPS cells are indistinguishable from hES cells in many aspects, including morphology, proliferation, feeder dependence, surface markers, gene expression, promoter activities, telomerase activities, in vitro differentiation, and teratoma formation. The four retroviruses are nearly completely silenced in human iPS cells, indicating that these cells are fully reprogrammed and do not depend on continuous expression of the transgenes for self-renewal.

hES cells are different from mouse counterparts in many respects (Rao, M., Dev. Biol. 275:269-286, 2004). hES cell colonies are flatter and do not override each other. hES cells depend on bFGF for self renewal (Amit et al. Dev. Biol. 227:271-78, 2000), whereas mouse ES cells depend on the LIF/Stat3 pathway (Matsuda et al. EMBO J 18:4261-69, 1999; Niwa et al. Genes Dev. 12:2048-60, 1998). BMP induces differentiation in hES cells (Xu et al. Nat. Methods 2:185-90, 2005) but is involved in self renewal of mouse ES cells (Ying et al. Cell 115:281-92, 2003). Because of these differences, it has been speculated that factors required for reprogramming might differ between humans and mice. On the contrary, our data show that the same four transcription factors induce iPS cells in both humans and mouse. The four factors, however, could not induce human iPS cell colonies when fibroblasts were kept under the culture condition for mouse ES cells after retroviral transduction (See Example 13, above), even though these cells stained positive for alkaline phosphatase. These data suggest that the fundamental transcriptional network governing pluripotency is common in human and mice, but extrinsic factors and signals maintaining pluripotency are unique for each species.

Deciphering of the mechanism by which the four factors induce pluripotency in somatic cells remains elusive. The function of Oct3/4 and Sox2 as core transcription factors to determine pluripotency is well documented (Boyer et al. Cell 122:947-956, 2005; Loh et al. Nat Genet 38:431-440, 2006; Wang et al. Nature 444:364-368, 2006). They synergistically upregulate “sternness” genes, while suppressing differentiation-associated genes in both mouse and human ES cells. However, they cannot bind their targets genes in differentiated cells, because of other inhibitory mechanisms, including DNA methylation. It may be speculated that c-Myc and Klf4 modifies chromatin structure so that Oct3/4 and Sox2 can bind to their targets (Yamanaka, Cell Stem Cell 1:39-49, 2007). Notably, Klf4 interacts with p300 histone acetyltransferase and regulates gene transcription by modulating histone acetylation (Evans et al. J Biol Chem, 2007).

The negative role of c-Myc in the self renewal of hES cells has also been reported (Sumi et al. Oncogene 26:5564-5576, 2007). They showed that forced expression of c-Myc induced differentiation and apoptosis of human ES cells. During iPS cell generation, transgenes derived from retroviruses are silenced when the transduced fibroblasts acquire ES-like state. The role of c-Myc in establishing iPS cells may be as a booster of reprogramming, rather than a controller of maintenance of pluripotency.

It has been found that each iPS clone contained 3-6 retroviral integrations for each factor. Thus, each clone had more than 20 retroviral integration sites in total, which may increase the risk of tumorigenesis. In the case of mouse iPS cells, ~20% of chimera mice and their offspring derived from iPS cells developed tumors Okita et al. Nature 448:313-17, 2007). This issue must be overcome to use iPS cells in human therapies. Therefore, non-retroviral methods to introduce the four factors, such as adenoviruses or cell-permeable recombinant proteins, are also contemplated as part of the invention. Alternatively, small molecules may replace the four factors for the induction of iPS cells.

### Experimental Procedures for Examples 14-20.

### Cell Culture

HDFs from facial dermis of 36-year-old Caucasian female and HFLS from synovial tissue of 69-year-old Caucasian male were purchased from Cell Applications, Inc. BJ fibroblasts from neonatal foreskin and NTERA-2 clone D1 human embryonic carcinoma cells were obtained from American Type Culture Collection. Human fibroblasts, NTERA-2, PLAT-E, and PLAT-A cells were maintained in Dulbecco's modified eagle medium (DMEM, Nacalai Tesque, Japan) containing 10% fetal bovine serum (FBS, Japan Serum) and 0.5% penicillin and streptomycin (Invitrogen). 293 FT cells were maintained in DMEM containing 10% FBS, 2 mM L-glutamine (Invitrogen), 1×10⁻⁴ M nonessential amino acids (Invitrogen), 1 mM sodium pyruvate (Sigma) and 0.5% penicillin and streptomycin. PA6 stroma cells (RIKEN Bioresource Center, Japan) were maintained in a-MEM containing 10% FBS and 0.5% penicillin and streptomycin. iPS cells were generated and maintained in Primate ES medium (ReproCELL, Japan) supplemented with 4 ng/ml recombinant human basic fibroblast growth factor (bFGF, WAKO, Japan). For passaging, human iPS cells were washed once with PBS and then incubated with DMEM/F12 containing 1 mg/ml collagenase IV (Invitrogen) at 37° C. When colonies at the edge of the dish started dissociating from the bottom, DMEF/F12/collangenase was removed and washed with hES cell medium. Cells were scraped and collected into 15 ml conical tube. An appropriate volume of the medium was added, and the contents were transferred to a new dish on SNL feeder cells. The split ratio was routinely 1:3. For feeder-free culture of iPS cells, the plate was coated with 0.3 mg/ml Matrigel (growth-factor reduced, BD Biosciences) at 4° C. overnight. The plate was warmed to room temperature before use. Unbound Matrigel was aspirated off and washed out with DMEM/F12. iPS cells were seeded on Matrigel-coated plate in MEF-CM or ES medium, both supplemented with 4 ng/ml bFGF. The medium was changed daily. For preparation of MEF-CM, MEFs derived from embryonic day 13.5 embryo pool of ICR mice were plated at 1×10⁶ cells per 100 mm dish and incubated overnight. Next day, the cells were washed once with PBS and cultured in 10 ml of ES medium. Twenty-four h after incubation, the supernatant of MEF culture was collected, filtered through a 0.22 μm pore-size filter, and stored at −20° C. until use.

### Plasmid Construction

The open reading frame of human OCT3/4 was amplified by RT-PCR and cloned into pCR2.1-TOPO. An EcoRI fragment of pCR2.1-hOCT3/4 was introduced into the EcoRI site of pMXs retroviral vector. To discriminate each experiment, a 20-bp random sequence, designated N20 barcode, was introduced into the NotI/SalI site of Oct3/4 expression vector. A unique ‘barcode sequence was used in each experiment to avoid inter-experimental contamination. The open reading frames of human SOX2, KLF4, and c-MYC were also amplified by RT-PCR and subcloned into pENTR-D-TOPO (Invitrogen). All of the genes subcloned into pENTR-D-TOPO were transferred to pMXs by using the Gateway cloning system (Invitrogen), according to the manufacturer's instructions. Mouse Slc7a1 ORF was also amplified, subcloned into pENTR-D-TOPO, and transferred to pLenti6/UbCNS-DEST (Invitrogen) by the Gateway system. The regulatory regions of the human OCT3/4 gene and the REX1 gene were amplified by PCR and subcloned into pCRXL-TOPO (Invitrogen). For phOCT4-Luc and phREX1-Luc, the fragments were removed by KpnI/BglII digestion from pCRXL vector and subcloned into the KpnI/BglII site of pGV-BM2. For pPolII-Luc, an AatII (blunted)/NheI fragment of pQBI-polII was inserted into the KpnI (blunted)/NheI site of pGV-BM2. All of the fragments were verified by sequencing. Primer sequences are shown in TABLE 13.

TABLE 13
Primer Sequences

| Primer | SEQ ID NO: | Sequence (5′ to 3′) | Applications |
|---|---|---|---|
| hOCT3/4-S944 | 26 | CCC CAG GGC CCC ATT | OCT3/4 Tg PCR |
| | | TTG GTA CC | |
| hSOX2-S691 | 27 | GGC ACC CCT GGC ATG | SOX2 Tg PCR |
| | | GCT CTT GGC TC | |
| hKLF4-S1128 | 28 | ACG ATC GTG GCC CCG | KLF4 endo and Tg |
| | | GAA AAG GAC C | PCR |
| hMYC-S1011 | 29 | CAA CAA CCG AAA ATG | c-MYC Tg PCR |
| | | CAC CAG CCC CAG | |
| pMXs-AS3200 | 30 | TTA TCG TCG ACC ACT | Tg PCR |
| | | GTG CTG CTG | |
| pMXs-L3205 | 31 | CCC TTT TTC TGG AGA | Tg PCR |
| | | CTA AAT AAA | |
| hOCT3/4-S1165 | 32 | GAC AGG GGG AGG GGA | Endo OCT3/4 |
| | | GGA GCT AGG | RT-PCR |
| hOCT3/4-AS1283 | 33 | CTT CCC TCC AAC CAG | |
| | | TTG CCC CAA AC | |
| hSOX2-S1430 | 34 | GGG AAA TGG GAG GGG | Endo SOX2 |
| | | TGC AAA AGA GG | RT-PCR |
| hSOX2-AS1555 | 35 | TTG CGT GAG TGT GGA | |
| | | TGG GAT TGG TG | |
| ECAT4-macaca- | 36 | CAG CCC CGA TTC TTC | NANOG RT-PCR |
| 968S | | CAC CAG TCC C | |
| ECAT4-macaca- | 37 | CGG AAG ATT CCC AGT | |
| 1334AS | | CGG GTT CAC C | |
| hGDF3-S243 | 38 | CTT ATG CTA CGT AAA | GDF3 RT-PCR |
| | | GGA GCT GGG | |
| hGDF3-AS850 | 39 | GTG CCA ACC CAG GTC | |
| | | CCG GAA GTT | |
| hREXI-RT-U | 40 | CAG ATC CTA AAC AGC | REX1 RT-PCR |
| | | TCG CAG AAT | |
| hREXI-RT-L | 41 | GCG TAC GCA AAT TAA | |
| | | AGT CCA GA | |
| hFGF4-RT-U | 42 | CTA CAA CGC CTA CGA | FGF4 RT-PCR |
| | | GTC CTA CA | |
| hFGF4-RT-L | 43 | GTT GCA CCA GAA AAG | |
| | | TCA GAG TTG | |
| hpH34-S40 | 44 | ATA TCC CGC CGT GGG | ESG1 RT-PCR |
| | | TGA AAG TTC | |
| hpH34-AS259 | 45 | ACT CAG CCA TGG ACT | |
| | | GGA GCA TCC | |
| hECAT15-1-S532 | 46 | GGA GCC GCC TGC CCT | DPPA4 RT-PCR |
| | | GGA AAA TTC | |
| hECAT15-1-AS916 | 47 | TTT TTC CTG ATA TTC | |
| | | TAT TCC CAT | |
| hECAT15-2-S85 | 48 | CCG TCC CCG CAA TCT | DPPA2 RT-PCR |
| | | CCT TCC ATC | |
| hECAT15-2-AS667 | 49 | ATG ATG CCA ACA TGG | |
| | | CTC CCG GTG | |
| hTERT-S3234 | 50 | CCT GCT CAA GCT GAC | hTERT RT-PCR |
| | | TCG ACA CCG TG | |
| hTERT-AS3713 | 51 | GGA AAA GCT GGC CCT | |
| | | GGG GTG GAG C | |
| hKLF4-AS1826 | 52 | TGA TTG TAG TGC TTT | Endo KLF4 |
| | | CTG GCT GGG CTC C | RT-PCR |
| hMYC-S253 | 53 | GCG TCC TGG GAA GGG | Endo c-MYC |
| | | AGA TCC GGA GC | RT-PCR |
| hMYC-AS555 | 54 | TTG AGG GGC ATC GTC | |
| | | GCG GGA GGC TG | |
| hMSX1-S665 | 55 | CGA GAG GAC CCC GTG | MSX1 RT-PCR |
| | | GAT GCA GAG | |
| hMSX1-AS938 | 56 | GGC GGC CAT CTT CAG | |
| | | CTT CTC CAG | |
| hBRACHYURY- | 57 | GCC CTC TCC CTC CCC | BRACHYURY/T |
| S1292 | | TCC ACG CAC AG | RT-PCR |
| hBRACHYURY- | 58 | CGG CGC CGT TGC TCA | |
| AS1540 | | CAG ACC ACA GG | |
| hGFAP-S1040 | 59 | GGC CCG CCA CTT GCA | GFAP RT-PCR |
| | | GGA GTA CCA GG | |
| hGFAP-AS1342 | 60 | CTT CTG CTC GGG CCC | |
| | | CTC ATG AGA CG | |
| hPAX6-S1206 | 61 | ACC CAT TAT CCA GAT | PAX6 RT-PCR |
| | | GTG TTT GCC CGA G | |
| hPAX6-AS1497 | 62 | ATG GTG AAG CTG GGC | |
| | | ATA GGC GGC AG | |
| hFOXA2-S208 | 63 | TGG GAG CGG TGA AGA | FOXA2 RT-PCR |
| | | TGG AAG GGC AC | |
| hFOXA2-AS398 | 64 | TCA TGC CAG CGC CCA | |
| | | CGT ACG ACG AC | |
| hSOX17-S423 | 65 | CGC TTT CAT GGT GTG | SOX17 RT-PCR |
| | | GGC TAA GGA CG | |
| hSOX17-AS583 | 66 | TAG TTG GGG TGG TCC | |
| | | TGC ATG TGC TG | |
| hAADC-S1378 | 67 | CGC CAG GAT CCC CGC | AADC RT-PCR |
| | | TTT GAA ATC TG | |
| hAADC-AS1594 | 68 | TCG GCC GCC AGC TCT | |
| | | TTG ATG TGT TC | |
| hChAT-S1360 | 69 | GGA GGC GTG GAG CTC | ChAT RT-PCR |
| | | AGC GAC ACC | |
| hChAT-AS1592 | 70 | CGG GGA GCT CGC TGA | |
| | | CGG AGT CTG | |
| hMAP2-S5401 | 71 | CAG GTG GCG GAC GTG | MAP2 RT-PCR |
| | | TGA AAA TTG AGA GTG | |
| hMAP2-AS5587 | 72 | CAC GCT GGA TCT GCC | |
| | | TGG GGA CTG TG | |
| hDAT-S1935 | 73 | ACA GAG GGG AGG TGC | SLC6A3/DAT |
| | | GCC AGT TCA CG | RT-PCR |
| hDAT-AS2207 | 74 | ACG GGG TGG ACC TCG | |
| | | CTG CAC AGA TC | |
| hLMX1B-S770 | 75 | GGC ACC AGC AGC AGC | LMX1B RT-PCR |
| | | AGG AGC AGC AG | |
| hLMXIB-AS1020 | 76 | CCA CGT CTG AGG AGC | |
| | | CGA GGA AGC AG | |
| hMYL2A-S258 | 77 | GGG CCC CAT CAA CTT | MYL2A RT-PCR |
| | | CAC CGT CTT CC | |
| hMYL2A-AS468 | 78 | TGT AGT CGA TGT TCC | |
| | | CCG CCA GGT CC | |
| hTnTc-S524 | 79 | ATG AGC GGG AGA AGG | TnTc RT-PCR |
| | | AGC GGC AGA AC | |
| hTnTc-AS730 | 80 | TCA ATG GCC AGC ACC | |
| | | TTC CTC CTC TC | |
| hMEF2C-S1407 | 81 | TTT AAC ACC GCC AGC | MEF2C RT-PCR |
| | | GCT CTT CAC CTT G | |
| hMEF2C-AS1618 | 82 | TCG TGG CGC GTG TGT | |
| | | TGT GGG TAT CTC G | |
| hMYHCB-S5582 | 83 | CTG GAG GCC GAG CAG | MYHCB RT-PCR |
| | | AAG CGC AAC G | |
| hMYHCB-AS5815 | 84 | GTC CGC CCG CTC CTC | |
| | | TGC CTC ATC C | |
| dT20 | 85 | TTT TTT TTT TTT TTT TTT | Reverse |
| | | TT | transcription |
| hMYC-S857 | 86 | GCC ACA GCA AAC CTC | Southern blot |
| | | CTC ACA GCC CAC | probe |
| hMYC-AS1246 | 87 | CTC GTC GTT TCC GCA | |
| | | ACA AGT CCT CTT C | |
| hOCT3/4-S | 88 | CAC CAT GGC GGG ACA | OCT3/4 cloning |
| | | CCT GGC TTC AG | |
| hOCT3/4-AS | 89 | ACC TCA GTT TGA ATG | |
| | | CAT GGG AGA GC | |
| hSOX2-S | 90 | CAC CAT GTA CAA CAT | SOX2 cloning |
| | | GAT GGA GAC GGA GCT | |
| | | G | |
| hSOX2-AS | 91 | TCA CAT GTG TGA GAG | |
| | | GGG CAG TGT GC | |
| hKLF4-S | 92 | CAC CAT GGC TGT CAG | KLF4 cloning |
| | | TGA CGC GCT GCT CCC | |
| hKLF4-AS | 93 | TTA AAA ATG TCT CTT | |
| | | CAT GTG TAA GGC GAG | |
| hMYC-S | 94 | CAC CAT GCC CCT CAA | c-MYC cloning |
| | | CGT TAG CTT CAC CAA | |
| hMYC-AS | 95 | TCA CGC ACA AGA GTT | |
| | | CCG TAG CTG TTC AAG | |
| Slc7a1-S | 96 | CAC CAT GGG CTG CAA | Mouse Slc7a1 |
| | | AAA CCT GCT CGG | cloning |
| Slc7a1-AS | 97 | TCA TTT GCA CTG GTC | |
| | | CAA GTT GCT GTC | |
| hREX1-pro5K-S | 98 | ATT GTC GAC GGG GAT | Promoter cloning |
| | | TTG GCA GGG TCA CAG | |
| | | GAC | |
| hREXx1-pro5K- | 99 | CCC AGA TCT CCA ATG | |
| AS | | CCA CCT CCT CCC AAA | |
| | | CG | |
| hOCT3/4-pro5K- | 100 | CACTCG AGG TGG AGG | |
| S | | AGC TGA GGG CAC TGT | |
| | | GG | |
| hOCT3/4-pro5K- | 101 | CAC AGA TCT GAA ATG | |
| AS | | AGG GCT TGC GAA GGG | |
| | | AC | |
| mehREX1-F1-S | 102 | GGT TTA AAA GGG TAA | Bisulfite |
| | | ATG TGA TTA TAT TTA | sequencing |
| mehREX1-F1-AS | 103 | CAA ACT ACA ACC ACC | |
| | | CAT CAA C | |
| mehOCT3/4 F2-S | 104 | GAG GTT GGA GTA GAA | |
| | | GGA TTG TTT TGG TTT | |
| mehOCT3/4 F2- | 105 | CCC CCC TAA CCC ATC | |
| AS | | ACC TCC ACC ACC TAA | |
| mehNANOG-FI-S | 106 | TGG TTA GGT TGG TTT | |
| | | TAA ATT TTT G | |
| mehNANOG-FI- | 107 | AAC CCA CCC TTA TAA | |
| AS | | ATT CTC AAT TA | |

### Lentivirus Production and Infection

293 FT cells (Invitrogen) were plated at 6×10⁶ cells per 100 mm dish, and incubated overnight. Cells were transfected with 3 μg of pLenti6/UbC-Slc7a1 along with 9 μg of Virapower packaging mix by Lipofectamine 2000 (Invitrogen), according to the manufacturer's instructions. Forty-eight hours after transfection, the supernatant of transfectant was collected and filtered through a 0.45 μm pore-size cellulose acetate filter (Whatman). Human fibroblasts were seeded at 8×10⁵ cells per 100 mm dish 1 day before transduction. The medium was replaced with virus-containing supernatant supplemented with 4 μg/ml polybrene (Nacalai Tesque), and incubated for 24 hours.

### Retroviral Infection and iPS Cell Generation

PLAT-E packaging cells were plated at 8×10⁶ cells per 100 mm dish and incubated overnight. Next day, the cells were transfected with pMXs vectors with Fugene 6 transfection reagent (Roche). Twenty-four hours after transfection, the medium was collected as the first virus-containing supernatant and replaced with a new medium, which was collected after 24 hours as the second virus-containing supernatant. Human fibroblasts expressing mouse Slc7a1 gene were seeded at 8×10⁵ cells per 100 mm dish 1 day before transduction. The virus-containing supernatants were filtered through a 0.45-μm pore-size filter, and supplemented with 4 μg/ml polybrene. Equal amounts of supernatants containing each of the four retroviruses were mixed, transferred to the fibroblast dish, and incubated overnight. Twenty-four hours after transduction, the virus-containing medium was replaced with the second supernatant. Six days after transduction, fibroblasts were harvested by trypsinization and re-plated at 5×10⁴ cells per 100-mm dish on an SNL feeder layer. Next day, the medium was replaced with hES medium supplemented with 4 ng/ml bFGF. The medium was changed every other day. Thirty days after transduction, colonies were picked up and transferred into 0.2 ml of hES cell medium. The colonies were mechanically dissociated to small clumps by pipeting up and down. The cell suspension was transferred on SNL feeder in 24-well plates. This stage was defined as passage 1.

### RNA Isolation and Reverse Transcription

Total RNA was purified with Trizol reagent (Invitrogen) and treated with Turbo DNA-free kit (Ambion) to remove genomic DNA contamination. One microgram of total RNA was used for reverse transcription reaction with ReverTraAce-α (Toyobo, Japan) and dT₂oprimer, according to the manufacturer's instructions. PCR was performed with ExTaq (Takara, Japan). Quantitative PCR was performed with Platinum SYBR Green qPCR Supermix UDO (Invitrogen) and analyzed with the 7300 real-time PCR system (Applied Biosystems). Primer sequences are shown in TABLE 13.

### Phosphatase Staining and Immunocytochemistry

Alkaline phosphatase staining was performed using the Leukocyte Alkaline Phosphatase kit (Sigma). For immunocytochemistry, cells were fixed with PBS containing 4% paraformaldehyde for IO min at room temperature. After washing with PBS, the cells were treated with PBS containing 5% normal goat or donkey serum (Chemicon), 1% bovine serum albumin (BSA, Nacalai tesque), and 0.1% Triton X-100 for 45 min at room temperature. Primary antibodies included SSEA1 (1:100, Developmental Studies Hybridoma Bank), SSEA3 (1:10, a kind gift from Dr. Peter W. Andrews), SSEA4 (1:100, Developmental Studies Hybridoma Bank), TRA-2-49/6E (1:20, Developmental Studies Hybridoma Bank), TRA-1-60 (1:50, a kind gift from Dr. Peter W. Andrews), TRA-1-81 (1:50, a kind gift from Dr. Peter W. Andrews), Nanog (1:20, AF1997, R&D Systems), βIII-tubulin (1:100, CB4 1 2, Chem icon), glial fibrillary acidic protein (1:500, 20334, DAKO), a-smooth muscle actin (pre-diluted, N1584, DAKO), desmin (1:100, RB-9014, Lab Vision), vimentin (1:100, SC-6260, Santa Cruz), α-fetoprotein (1:100, MAB1368, R&D Systems), tyrosine hydroxy lase (1:100, AB152, Chemicon). Secondary antibodies used were cyanine 3 (Cy3)-conjugated goat anti-rat IgM (1:500, Jackson Immunoresearch), Alexa546-conjugated goat anti-mouse IgM (1:500, Invitrogen), Alexa488-conjugated goat anti-rabbit IgG (1:500, Invitrogen), Alexa488-conjugated donkey anti-goat IgG (1:500, Invitrogen), Cy3-conjugated goat anti-mouse IgG (1:500, Chemicon), and Alexa488-conjugated goat anti-mouse IgG (1:500, Invitrogen). Nuclei were stained with 1 μg/ml Hoechst 33342 (Invitrogen).

### In Vitro Differentiation

For EB formation, human iPS cells were harvested by treating with collagenase IV. The clumps of the cells were transferred to poly(2-hydroxyethyl methacrylate)-coated dish in DMEM/F12 containing 20% knockout serum replacement (KSR, Invitrogen), 2 mM L-glutamine, 1×10⁻⁴ M nonessential amino acids, 1×10⁻⁴ M 2-mercaptoethanol (Invitrogen), and 0.5% penicillin and streptomycin. The medium was changed every other day. After 8 days as a floating culture, EBs were transferred to gelatin-coated plate and cultured in the same medium for another 8 days. Co-culture with PA6 was used for differentiation into dopaminergic neurons. PA6 cells were plated on gelatin-coated 6-well plates and incubated for 4 days to reach confluence. Small clumps of iPS cells were plated on PA6-feeder layer in Glasgow minimum essential medium (Invitrogen) containing 10% KSR (Invitrogen), 1×10⁻⁴ M nonessential amino acids, 1×10⁻⁴ M 2-mercaptoethanol (Invitrogen), and 0.5% penicillin and streptomycin. For cardiomyocyte differentiation, iPS cells were maintained on Matrigel-coated plate in MEF-CM supplemented with 4 ng/ml bFGF for 6 days. The medium was then replaced with RPMI1640 (Invitrogen) plus B27 supplement (Invitrogen) medium (RPMI/827), supplemented with 100 ng/ml human recombinant activin A (R & D Systems) for 24 hours, followed by 10 ng/ml human recombinant bone morphologenic protein 4 (BMP4, R&D Systems) for 4 days. After cytokine stimulation, the cells were maintained in RPMI/827 without any cytokines. The medium was changed every other day.

### Bisulfite Sequencing

Genomic DNA (1 μg) was treated with CpGenome DNA modification kit (Chemicon), according to the manufacturer's recommendations. Treated DNA was purified with QIAquick column (QIAGEN). The promoter regions of the human Oct3/4, Nanog, and Rex1 genes were amplified by PCR. The PCR products were subcloned into pCR2.1-TOPO. Ten clones of each sample were verified by sequencing with the M13 universal primer. Primer sequences used for PCR amplification were provided in TABLE 13.

### Luciferase Assay

Each reporter plasmid (1 μg) containing the firefly luciferase gene was introduced into human iPS cells or HDF with 50 ng of pRL-TK (Promega). Forty-eight hours after transfection, the cells were lysed with IX passive lysis buffer (Promega) and incubated for 15 min at room temperature. Luciferase activities were measured with a Dual-Luciferase reporter assay system (Promega) and Centro LB 960 detection system (BERTHOLD), according to the manufacturer's protocol.

### Teratoma Formation

The cells were harvested by collagenase IV treatment, collected into tubes and centrifuged, and the pellets were suspended in DMEM/F12. One quarter of the cells from a confluent 100 mm dish was injected subcutaneously to dorsal flank of a SCID mouse (CREA, Japan). Nine weeks after injection, tumors were dissected, weighted, and fixed with PBS containing 4% paraformaldehyde. Paraffin-embedded tissue was sliced and stained with hematoxylin and eosin.

### Western Blotting

The cells at semiconfluent state were lysed with RIPA buffer (50 mM Tris-HCl, pH 8.0, 150 mM NaCl, 1% Nonidet P-40 (NP-40), 1% sodium deoxycholate, and 0.1% SDS), supplemented with protease inhibitor cocktail (Roche). The cell lysate of MEL-I hES cell line was purchased from Abeam. Cell lysates (20 μg) were separated by electrophoresis on 8% or 12% SDS-polyacrylamide gel and transferred to a polyvinylidine ditluoride membrane (Millipore). The blot was blocked with TBST (20 mM Tris-HCl, pH 7.6, 136 mM NaCl, and 0.1% Tween-20) containing 1% skim milk and then incubated with primary antibody solution at 4° C. overnight. After washing with TBST, the membrane was incubated with horseradish peroxidase (HRP)-conjugated secondary antibody for 1 hour at room temperature. Signals were detected with Immobilon Western chemiluminescent HRP substrate (Millipore) and LAS3000 imaging system (FUJIFILM, Japan). Antibodies used for western blotting were anti-Oct3/4 (1:600, SC-5279, Santa Cruz), anti-Sox2 (1:2000, AB5603, Chemicon), anti-Nanog (1:200, R&D Systems), anti-Klf4 (1:200, SC-20691, Santa Cruz), anti-c-Myc (1:200, SC-764, Santa Cruz), anti-E-cadherin (1:1000, 610182, BD Biosciences), anti-Dppa4 (1:500, ab31648, Abeam), anti-FoxD3 (1:200, AB5687, Chemicon), anti-telomerase (1:1000, ab23699, Abeam), anti-Sall4 (1:400, ab29112, Abeam), anti-LIN-28 (1:500, AF3757, R&D systems), anti-P-actin (1:5000, A5441, Sigma), anti-mouse lgG-HRP (1:3000, #7076, Cell Signaling), anti-rabbit IgG-HRP (1:2000, #7074, Cell Signaling), and anti-goat lgG-HRP (1:3000, SC-2056, Santa Cruz).

### Southern Blotting

Genomic DNA (5 μg) was digested with BglII, EcoRI, and Ncol overnight. Digested DNA fragments were separated on 0.8% agarose gel and transferred to a nylon membrane (Amersham). The membrane was incubated with digoxigenin (DIG)-labeled DNA probe in DIG Easy Hyb buffer (Roche) at 42° C. overnight with constant agitation. After washing, alkaline phosphatase-conjugated anti-DIG antibody (1:10,000, Roche) was added to a membrane. Signals were raised by CDP-star (Roche) and detected by LAS3000 imaging system.

### Short Tandem Repeat Analysis and Karyotyping

The genomic DNA was used for PCR with Powerplex 16 system (Promega) and analyzed by ABI PRISM 3100 Genetic analyzer and Gene Mapper v3.5 (Applied Biosystems). Chromosomal G-band analyses were performed at the Nihon Gene Research Laboratories, Japan.

### Detection of Telomerase Activity

Telomerase activity was detected with a TRAPEZE telomerase detection kit (Chemicon), according to the manufacturer's instructions. The samples were separated by TBE-based 10% acrylamide non-denaturing gel electrophoresis. The gel was stained with SYBR Gold (1:10,000, Invitrogen).

Example 21: Generation of Induced Pluripotent Stem Cells without Myc

The direct reprogramming of somatic cells is considered to provide an opportunity to generate patient- or disease-specific pluripotent stem cells. Such pluripotent stem (iPS) cells are induced from mouse fibroblasts by the retroviral transduction of four transcription factors, Oct3/4, Sox2, Klf4, and c-Myc (Takahashi et al. Ce/1126:663-76, 2006). Mouse iPS cells are indistinguishable from mouse ES cells in many aspects and give rise to germline-competent chimeras (Wemig et al. Nature 448:318-24, 2007; Okita et al. Nature 448:313-17, 2007; Mahernli et al. Cell Stem Cell 1:55-70, 2007). It was noted above that each iPS clone contained 3-6 retroviral integrations for each factor. Thus, each clone had more than 20 retroviral integration sites in total, which may increase the risk of tumorigenesis. In the case of mouse iPS cells, ~20% of chimera mice and their offspring derived from iPS cells developed tumors (Okita et al. Nature 448:313-17, 2007). In particular, it was found that the reactivation of the c-Myc retrovirus results in an increased incidence of tumor formation in the chimeras and progeny mice generated with mouse iPS cells, thus hindering the clinical application of this technology (Okita et al. Nature 448:313-17, 2007). Therefore, a modified protocol for the induction of iPS cells, which does not require the Myc retrovirus was developed. With this new protocol, significantly fewer non-iPS background cells were obtained. Furthermore, the iPS cells generated without Myc were constantly of high quality. These findings are important for the future clinical application of this iPS cell technology.

Substitution of the Four Factors with Other Family Members

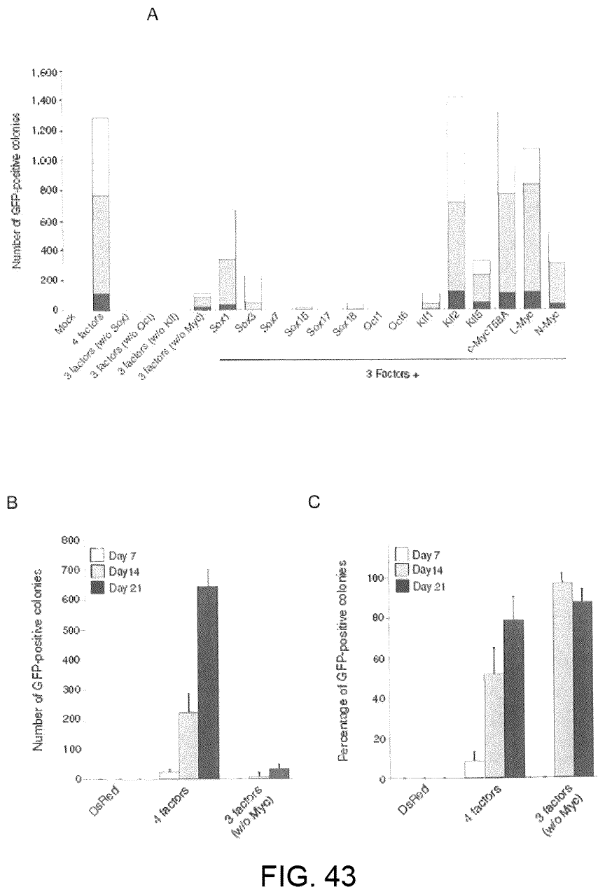

This study was initiated to examine whether the family proteins of the four factors could also induce iPS cells. In other words, further investigations were performed to assess which family members of a given gene family could substitute for others as nuclear reprogramming factors. Mouse embryonic fibroblasts (MEF) containing a GFP-IRES-Puror transgene driven by the Nanog gene regulatory elements were used (Okita et al. Nature 448:313-17, 2007). Nanog is specifically expressed in mouse ES cells and preimplantation embryos (Chambers et al. Cell 113:643-55, 2003; Mitsui et al. Cell 113:631-42, 2003) and can serve as a selection marker during iPS cell induction. By introducing the aforementioned four factors, iPS cells are induced as GFP-expressing colonies. Nanog-selected iPS cells are indistinguishable from ES cells and have been shown to give rise to germline-competent chimeras (Wemig et al. Nature 448:318-24, 2007; Okita et al. Nature 448:313-17, 2007; Maherali et al. Cell Stem Cell 1:55-70, 2007). Oct3/4 belongs to the Oct family transcription factors, which contain the POU domain (Ryan et al. Genes Dev 11:1207-25, 1997). The closest homologs of Oct3/4 are Oct1 and Oct6. Oct3/4, Oct1, or Oct6 were introduced together with the remaining three factors, into the Nanog-reporter MEF by retroviruses. With Oct3/4, many GFP-positive colonies were observed (FIG. 43A). In contrast, no GFP-positive colonies were obtained with Oct1 or Oct6, thus indicating the inability of these two homologs to induce iPS cells.

Sox2 belongs to the Sox (SRY-reiated HMG-box) transcription factors, characterized by the presence of the high mobility group (HMG) domain (Schepers et al. Dev Cell 3:167-70, 2002). Sox1, Sox3, Sox7, Sox15, Sox17, and Sox18 were tested and GFP-positive colonies were obtained with Sox1. In addition, fewer GFP-positive colonies were obtained with Sox3, Sox15, and Sox18 (FIG. 43A). Sox18, however, failed to expand the cells.

Klf4 belongs to Krilppel-like factors (Klfs), zinc-finger proteins that contain amino acid sequences that resemble those of the Drosophila embryonic pattern regulator Krilppel (Dang et al. Int J Biochem Cell Biol 32:1103-21, 2000). Klf1, Klf2, and Klf5 were tested and GFP-expressing colonies with Klf2 were thus obtained (FIG. 43A). Klf1 and Klf5 were also capable of inducing iPS cells, but with a lower efficiency.

c-Myc has two related proteins, N-Myc and L-Myc (Adhikary et al. Nat Rev Mol Cell Biol 6:635-45, 2005). GFP-positive colonies emerged with both N-Myc and L-Myc (FIG. 43A). Therefore, some, but not all family proteins of the four factors can induce iPS cells.

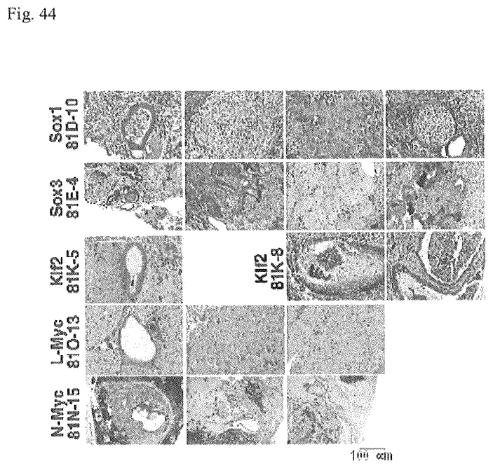

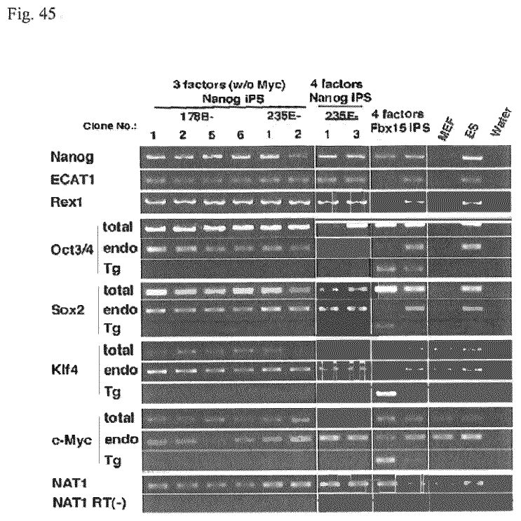

The family proteins were also tested for their ability to induce iPS cells from MEFs in which βgeo was knocked into the Fbx15 locus (Tokuzawa et al. Mol Cell Biol 23:2699-708, 2003). Similar results to those with the Nanog-based selection were obtained: Sox2 could be replaced by Sox1 and Sox3, Klf4 by Klf2, and c-Myc by N-Myc and L-Myc. The cells generated by the family proteins were expandable and showed a morphology indistinguishable from that of ES cells (not shown). They gave rise to teratomas in nude mice (FIG. 44). Therefore, some family proteins are capable of inducing iPS cells from both Nanog-reporter MEF an [symbol] Fbx15-reporter MEF.

As has been stated above, iPS cell generation from somatic cells was evaluated by optimizing retroviral transduction and subsequent culture conditions. Furthermore, optimization would be useful for the application of this iPS cell technology to human cells, especially in clinical situations. Unexpectedly, a few ES cell-like and GFP-positive colonies from Nanog-reporter MEF were obtained without any Myc retroviruses (FIG. 43A). This was in contrast to a previous study in which no GFP-positive colonies could be obtained with1;rnt c-Myc (Okita et al. Nature 448:313-17, 2007). Consistent with the efforts toward optimization, one difference between the two studies is the timing of the drug selection: In the previous study, puromycin selection was initiated seven days after the transduction, whereas in this experiment the selection was started at 14 days. This suggests that iPS cell induction without Myc is a slower process than that with Myc. Furthermore, as is further discussed herein, the omission of Myc resulted in a less efficient but more specific induction of iPS cells.

### Myc Omission Results in More Specific iPS Cell Induction

To test whether iPS cell induction without Myc is a slower process than that with Myc, Nanog-reporter MEFs were transduced with either the four factors or three factors devoid of Myc, and then puromycin selection was started seven, 14, or 21 days after the transduction (FIG. 43B). With the four factors, GFP-positive colonies were observed in all of the conditions. The colony numbers significantly increased when puromycin selection was delayed. Without Myc, no GFP-colonies were observed when selection was initiated seven days after the transduction. In contrast, GFP-positive colonies did emerge even without Myc when selection was started 14 or 21 days after the transduction. The colony numbers were fewer with the three factors than with the four factors in each condition. Nanog-selected iPS cells generated without Myc retroviruses expressed ES cell marker genes at comparable levels to those in ES cells (FIG. 45), and thus gave rise to adult chimeras when transplanted into blastocysts (TABLE 14).

TABLE 14
Summary of blastocysts injections

| | | | injected | | |
|---|---|---|---|---|---|
| iPS clones | origin-genotype* | selection | blastocysts | born mice | chimeras |
| 142B-6 | MEF-FB/gfp | G418 | 39 | 7 | 3 |
| 142B-12 | | | 46 | 12 | 5 |
| 178B-1 | MEF-Ng | Puro | 156 | 50 | 5 |
| 178B-2 | | | 142 | 43 | 17 |
| 178B-5 | | | 60 | 20 | 5 |
| 178B-6 | | | 28 | 10 | 4 |
| 256H-4 | TTF-ACTB- | No | 72 | 6 | 5 |
| 256H-13 | DsRed | | 96 | 8 | 5 |
| 256H-18 | | | 90 | 17 | 11 |

All iPS clones were induced with three factors devoid of Myc from MEF or TTF.
*FB, Fbx15-βgeo reporter; Ng, Nanog-GFP-IRES-Puro^{r} reporter; gfp, CAG-EGFP

Another difference is that fewer GFP-negative colonies as well as background cells were observed with the three factors devoid of Myc than with the four factors (FIG. 43C). Therefore, the omission of Myc resulted in a less efficient but more specific induction of iPS cells.

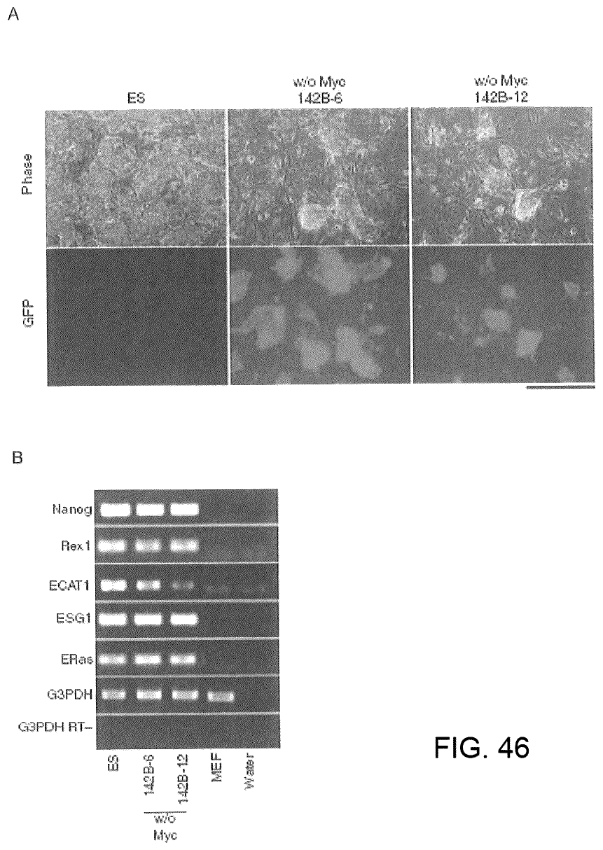

It was also possible to generate a few iPS cells without Myc from MEFs, in which βgeo was knocked into the Fbx15 locus (Tokuzawa et al. Mol. Cell Biol. 23:2699-708, 2003). (FIG. 46A). This is again in contrast to the original report, in which no iPS cells were obtained without c-Myc (Takahashi et al. Cell 126:663-76, 2006). In the two experiments, G418 selection was initiated with the same timing: three days after the transduction. However, the colonies were selected 14-21 days after the transduction in the previous report, whereas ~30 days were required in the current study. Another difference was that the retroviral transfection efficiency was raised by preparing each of the four or three factors separately in an independent Plat-E (Morita et al. Gene Ther. 7:1063-66, 2000) plate in this study. In comparison to the original work in which all the four factors were prepared in a single Plat-E plate, a significant increase in the number of iPS cell colonies was observed (not shown). This is consistent with the notion that iPS cell induction without Myc is a slower and less efficient process than that with Myc.

Fbx15-selected iPS cells, which were generated with the four factors, express lower levels of ES-cell marker genes than ES cells (Takahashi et al. Cell 126:663-76, 2006). They cannot produce adult chimeras when microinjected into blastocysts. In contrast, iPS cells generated without Myc expressed ES-cell marker genes at comparable levels to those in ES cells even with the Fbx15 selection (FIG. 46B). Furthermore, adult chimeras were obtained with high iPS cell contribution from these cells (FIG. 46C, TABLE 14). No increased incidence of tumor formation was observed in these chimeras. That is, six of 37 chimeras derived from iPS cells with the four factors died of tumors within 100 d after birth. By contrast, all 26 chimeras generated without Myc survived this period. Thus, the omission of the Myc retrovirus significantly reduced the risk of tumorigenicity in chimeras.

### iPS Cell Induction in the Absence of Drug Selection

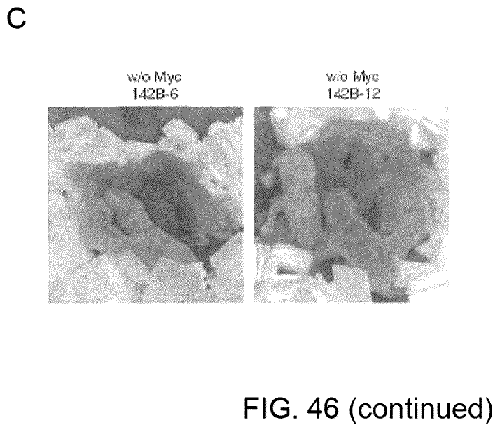

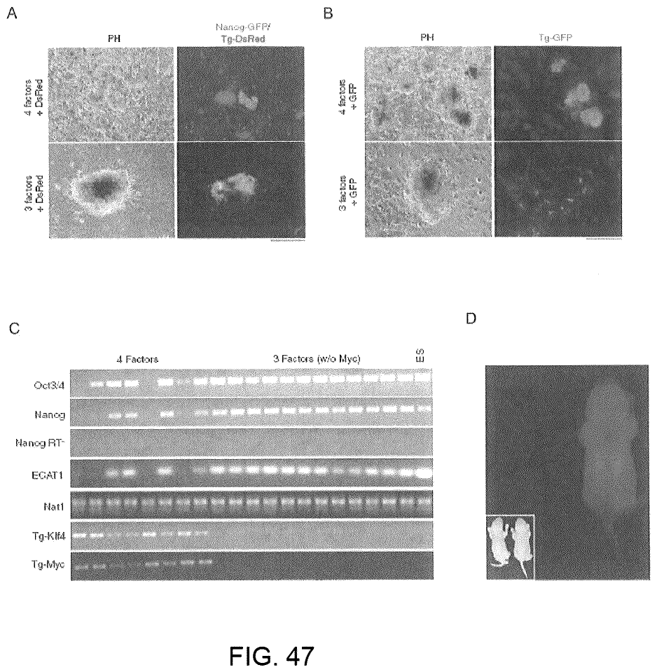

Next, it was determined whether the omission of Myc would result in efficient isolation of iPS cells without drug selection. The four or three factors were introduced into adult tail tip fibroblasts (TTF) containing the Nanog reporter, but puromycin selection was not applied. DsRed retrovirus was transduced together with the four or three factors to visualize transduced cells. Thirty days after the retroviral transduction, the dishes transduced with the four factors were covered with numerous GFP-negative colonies and background cells (FIG. 47A, TABLE 15).

TABLE 15
Summary of experiments (Nanog-GFP reporter TTF,
without selection)

| Experiment | | Cell | Total | | |
|---|---|---|---|---|---|
| Number | Factors | seeded | colonies | picked up | established |
| 220 | 4 | 5 × 10⁴ | many (107) | 26(24) | 25(22) |
| 256 | 4 | 5 × 10⁴ | many (4) | | |
| | 3 | 3.5 × 10⁵ | 7(4) | 7(4) | 6(5) |
| 272 | 4 | 5.4 × 10⁴ | many (132) | 6(6) | 5(4) |
| | 3 | 3.1 × 10⁵ | 21(8) | 4(4) | 2(2) |
| 309 | 4 | 2.3 × 10⁴ | many (424) | | |
| | 3 | 9.6 × 10⁵ | 43(24) | | |

Numbers in parentheses indicate number of colonies or clones that were positive for GFP.
The ratios of the retroviruses, Oct3/4, Sox2, Klf4, (c-Myc), and DsRed, were 1:1:1:(1):4 in experiment 256 and 1:1:1:(1):1 in experiments 272 and 309. In experiment 220, DsRed was not introduced.

Using fluorescent microscopy, small portions of these colonies (4, 132, and 424 colonies in three independent experiments) were found GFP-positive. Of note, the GFP-positive colonies were negative for DsRed, which was consistent with the retroviral silencing observed in Nanog-selected iPS cells (Okita et al. Nature 448:313-17, 2007). In contrast, with the three factors devoid of Myc, a small number (7, 21, and 43 in three independent experiments) of discrete colonies were observed with few background cells. Approximately a half of them expressed GFP in a patchy manner. DsRed was only detected in a small portion of some colonies, indicating that it was largely silenced. No overlap was observed between GFP and DsRed. Most of these colonies were expandable and produced iPS cells, which became positive for GFP and negative for DsRed at passage 2. Therefore, the omission of c-Myc resulted in more specific generation of iPS cells, in which Nanog-GFP is activated whereas the retroviruses are silenced.

Next, generation of iPS cells was attempted from adult TTF that did not have selection markers, but had the DsRed transgene driven by a constitutively active promoter (Vintersten et al. Genesis 40:241-46, 2004). The four factors or the three factors devoid of Myc were introduced. In addition, a GFP retrovirus was introduced to monitor silencing. After 30 days without drug selection, ~1000 colonies emerged from 0.5×10⁵ cells transduced with the four factors. Most of them were positive for GFP, indicating that retroviral silencing did not take place in these cells. In contrast, only 16 colonies (FIG. 47B) emerged from 3.5×10⁵ cells transduced with the three factors devoid of Myc. Most of these colonies express no GFP, while the remaining expressed GFP in small portions. All of these colonies were expandable and showed iPS- or ES-like morphology at the second passage. They were all negative for GFP, thus indicating retroviral silencing. RT-PCR showed that these cells expressed ES cell marker genes at comparable levels to those in ES cells (FIG. 47C). In addition, RT-PCR confirmed the retroviral silencing of Klf4 and the absence of the Myc transgene in iPS cells generated with the three factors. Furthermore, when transplanted into blastocysts, these cells gave rise to chimeras (FIG. 47D, TABLE 14). Therefore, by omitting Myc, good iPS cells can be efficiently generated from adult TTF without drug selection. These findings should be useful for the application of this iPS cell technology to human cells, especially in clinical situations.

Induction of Human iPS Cells without Myc Retroviruses

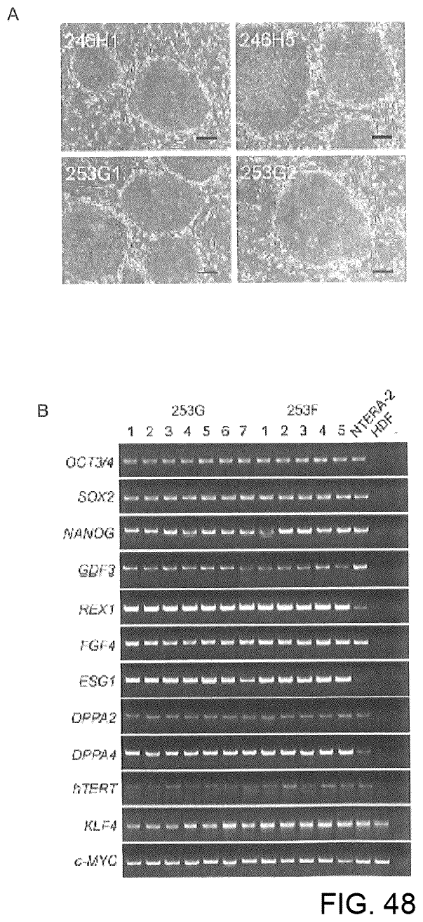

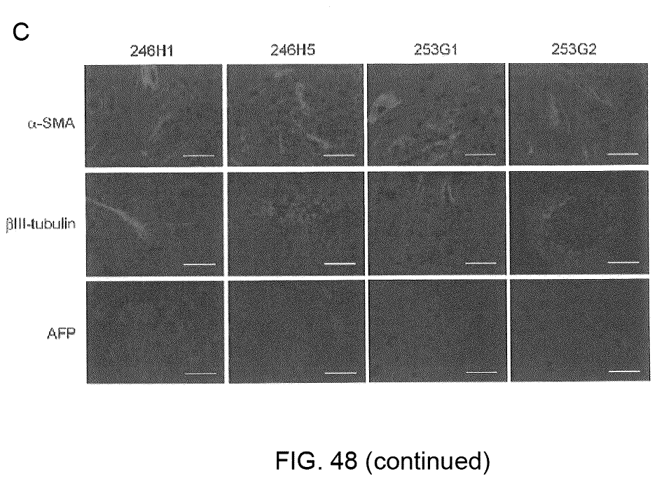

FIGS. 48(A)-(C) show induction of human iPS cells without Myc retroviruses. The retroviruses for Oct3/4, Sox2 and Kl f4 were introduced into BJ fibroblasts (246G) or HDF (253G). After 30 days, a few hES cell-like colonies emerged. These cells were expandable and showed hES cell-like morphology (FIG. 48(A)). Results were obtained for the expression of ES cell marker genes in human iPS cells derived from HDF without Myc retroviruses (2530) or with Myc (253F) (FIG. 48(8)), as were results for embryoid body-mediated differentiation of human iPS cells generated without Myc retroviruses (FIG. 48(C)).

### Experimental Procedures for Example 21.

Plasmid construction. The coding regions of family genes were amplified by RT-PCR with primers listed in TABLE 16, subcloned into pDONR201 or pENTR-D-TOPO (Invitrogen), and recombined with pMXs-gw by the LR reaction (Invitrogen).

TABLE 16
Primers used for cloning of the family factors

| Genes | Sequences | SEQ ID NO: |
|---|---|---|
| Sox1 | CAC CAT GTA CAG CAT GAT GAT GGA GAC CGA CCT | 108 |
| | CTA GAT ATG CGT CAG GGG CAC CGT GC | 109 |
| Sox3 | CAC CAT GTA CAG CCT GCT GGA GAC TGA ACT CAA | 110 |
| | TCA GAT GTG GGT CAG CGG CAC CGT TCC ATT | 111 |
| Sox7 | CAC CTC GGC CAT GGC CTC GCT GCT GGG | 112 |
| | CTC CAT TCC TCC AGC TCT ATG ACA CAC | 113 |
| Sox15 | CAC CAT GGC GCT GAC CAG CTC CTC ACA A | 114 |
| | TTA AAG GTG GGT TAC TGG CAT GGG | 115 |
| Sox17 | CAC CAG AGC CAT GAG CAG CCC GGA TG | 116 |
| | CGT CAA ATG TCG GGG TAG TTG CAA TA | 117 |
| Sox18 | CAC CAT GCA GAG ATC GCC GCC CGG CTA CG | 118 |
| | CTA GCC TGA GAT GCA AGC ACT GTA ATA GAC | 119 |
| Oct1 | CAC CAT GAA TAA TCC ATC AGA AAC CAA T | 120 |
| | GCT CTG CAC TCA GCT CAC TGT GCC | 121 |
| Oct6 | CAC CAT GGC CAC CAC CGG GCA GTA TCT G | 122 |
| | GGA ACC CAG TCC GCA GGG TCA CTG | 123 |
| Klf1 | CAC CAT GAG GCA GAA GAG AGA GAG GAG GC | 124 |
| | TCA GAG GTG ACG CTT CAT GTG CAG AGC TAA | 125 |
| Klf2 | CAC CAT GGC GCT CAG CGA GCC TAT CTT GCC | 126 |
| | CTA CAT ATG TCG CTT CAT GTG CAA GGC CAG | 127 |
| Klf5 | CAC CAT GCC CAC GCG GGT GCT GAC CAT G | 128 |
| | TCG CTC AGT TCT GGT GGC GCT TCA | 129 |
| L-MycWT | CAC CAT GGA CTT CGA CTC GTA TCA GCA CTA TTT C | 130 |
| | TTA GTA GCC ACT GAG GTA CGC GAT TCT CTT | 131 |
| N- | CAC CAT GCC CAG CTG CAC CGC GTC CAC CAT | 132 |
| MycWT | TTA GCA AGT CCG AGC GTG TTC GAT CT | 133 |

Retroviral transduction. pMXs-based retroviral vectors were transfected into Plat-E cells (Morita et al. Gene Ther. 7:1063-66, 2000) using Fugene 6 reagents (Roche) according to manufacturer's instruction. Twenty-four hours after transfection, the medium was replaced. After 24 hours, virus-containing supernatant were used for retroviral infection. In a “mixed” protocol, the mixture of plasmids for the four factors was transfected into a single dish of Plat-E cells. In a “separate” method, each plasmid was transfected into separate dishes of Plat-E cells. Virus-containing supernatant was mixed prior to transduction. Significantly higher transduction efficiency was observed with the separate method.

Induction of iPS cells with drug selection. The induction of iPS cells was performed as previously described (Takahashi et al. Cell 126:663-76, 2006; Okita et al. Nature 448:313-17, 2007) with some modifications. Briefly, MEFs, which contained either the Nanog-GFP-IRES-Puro^{r} reporter or the Fbx15-βgeo reporter, or both, were seeded at 1.3 and 8.0×10⁵ cells/well in 6-well plates and 100 mm dish, respectively, with SNL feeder cells (McMahon et al. Cell 62:1073-85, 1990). The transduced cells were cultivated with ES medium containing LIF (Meiner et al. Proc. Natl. Acad. Sci. U.S.A. 93:14041-46. (1996). Selection with G418 (300 μg/ml) or puromycin (1.5 μg/ml) was started as indicated. Twenty-five to 30 days after transduction, the number of colonies was recorded. Some colonies were then selected for expansion.

iPS cells induction without drug selection. TTFs were isolated from adult Nanog-reporter mice or adult DsRed-transgenic mice (Vintersten et al. Genesis 40:241-46, 2004). Retroviral-containing supernatant was prepared in the separated method. For the four-factor transduction, retrovirus-containing supernatants for Klf4, c-Myc, Oct3/4, Sox2 and DsRed, were mixed with the ratio of 1:1:1:1:4. When the three factors were transduced, retrovirus-containing supernatants for Klf4, Oct3/4, Sox2, Mock, and DsRed were mixed with the ratio of 1:1:1:1:4. With DsRed transgenic mice, the GFP retrovirus was used instead of DsRed. For transfection, TTFs were seeded at 8.0×10⁵ cells per 100-mm dishes, which did not have feeder cells. TTFs were incubated in the virus/polybrene-containing supernatants for 24 hours. Four days after transduction, TTFs transduced with the three factors were reseeded at 3.5×10⁵ cells per 100-mm dishes with SNL feeder cells and cultured with ES medium. TTFs transduced with the four factors were re-seeded at 0.5×10⁵ cells per 100-mm dishes with feeder cells. Thirty to 40 days after transduction, the colonies were selected for expansion.

Characterization of iPS cells. RT-PCR and teratoma formation were performed as previously described. For the chimera experiments, 15-20 iPS cells were injected into BDFI-derived blastocysts, which were then transplanted into the uteri of pseudo-pregnant mice.

Example 22: Establishment of Human iPS Cells from Epithelial Cells with Six Factors.

Among the nuclear reprogramming factors disclosed herein is a nuclear reprogramming factor comprising one or more gene products from the following genes: Klf4, c-Myc, Oct3/4, Sox2, Nanog, and Lin28 (NCBI accession number NM_145833 (mouse) and NM_024674 (human)). Establishment of induced pluripotent stem cells was performed with combinations of these gene products. The results are shown in TABLE 17.

TABLE 17
Summary of Experiments with Six Factors

| | Day 23 | | Day 29 | | Day 23 | |
|---|---|---|---|---|---|---|
| | non ES like | ES like | non ES like | ES like | non ES like | ES like |
| 6F | 59 | 39 | 167 | 42 | 16 | 27 |
| -L | 49 | 5 | 53 | 14 | | |
| -N | 220 | 11 | 216 | 47 | | |
| -M | 2 | 0 | 15 | 0 | | |
| -O | 0 | 0 | 0 | 0 | | |
| -S | 491 | 0 | 489 | 0 | | |
| -K | 61 | 0 | 51 | 0 | | |
| -KS | 1206 | 0 | 1305 | 0 | | |
| -KO | 0 | 0 | 0 | 0 | | |
| -KM | 0 | 0 | 0 | 0 | 0 | 0 |
| -KN | 51 | 0 | 57 | 0 | | |
| -KL | 28 | 0 | 41 | 0 | | |
| -SO | 0 | 0 | 0 | 0 | | |
| -SM | 0 | 0 | 0 | 0 | | |
| -SN | 188 | 0 | 171 | 0 | | |
| -SL | 112 | 0 | 136 | 0 | | |
| -OM | 0 | 0 | 0 | 0 | | |
| -ON | 0 | 0 | 0 | 0 | | |
| -OL | 0 | 0 | 0 | 0 | | |
| -MN | 3 | 0 | 8 | 0 | | |
| -ML | 0 | 0 | 0 | 0 | | |
| -NL | 98 | 1 | 119 | 9 | 17 | 6 |
| GFP | 0 | 0 | 0 | 0 | | |
| KO | 0 | 0 | 0 | 0 | | |
| KS | 0 | 0 | 0 | 0 | | |

6×10⁶ 293 FT cells were plated on 10 cm dish and cultured overnight, and then transfected with 3 μg of pLenti6/UbC-Slc7a1 lentiviral vector together with 9 μg of Virapower packaging mix by Lipofectamine 2000 (Invitrogen). After 24 hours, the culture medium was replaced with a fresh medium. After 20 hours, the culture supernatant was collected and filtrated through 0.45-μm pore-size cellulose acetate filter (Whatman). 5×10⁵ epithelial cells were prepared on the previous day. To the dish which the culture supernatant was removed from, the aforementioned filtrated culture supernatant containing viruses and 4 μg/ml polybrene (Nacalai Tesque) were added. Then, the cells were cultured for 24 hours.

In addition, 1.0×10⁶ Plat-E cells were plated on 6 cm dish and cultured. On the next day, the cells were transfected with 9.0 μg of pMX-based retrovirus vector incorporating Klf4, c-myc, oct3/4, sox2, nanog and/or Lin-28 by using 27 μI of Fugene6 transfection reagent. After 24 hours, the culture medium was replaced with a fresh medium. On the next day, the culture supernatant of Plat-E cells was collected and filtrated through 0.45-μm pore-size cellulose acetate filter (Whatman). Seven days after lentivirus infection, epithelial cells were plated at 3.0×10⁵ cells per 6 cm dish again, and the aforementioned culture supernatant containing retrovirus and polybrene were added thereto.

The term “6F” in TABLE 17 refers to the six factors (klf4, c-myc, oct3/4, sox2, nanog and Lin-28), the term “L” refers to Lin-28, the term “N” refers to nanog, the term “M” refers to c-Myc, the term “O” refers to Oct3/4, the term “S” refers to Sox2, and the term “K” refers to Klf4, respectively. The symbol “−” refers to colonies obtained by subtracting from the six factors those factors shown by the term subsequent to the symbol “−”. For example, the term “−L” shows the colonies obtained with the remaining five factors other than lin-28, and the term “−KS” shows the colonies obtained with the remaining four factors other than Klf4 and Sox2, respectively.

The numbers in TABLE 17 refer to the number of colonies. The term “non-ES like” refers to shows colonies having a non-ES cell like morphology, and the term “ES like” refers to colonies having an ES like cell morphology.

Two experimental results are shown. The first experiment shows the number of colonies from cells introduced with various combinations of factors 23 days or 29 days after gene introduction and the second experiment shows the number of “6F,” “−KM,” and “—NL.” According to these experimental results, the number of colonies from cells not-introduced with lin-28 such as “−L” was smaller than that of cell transduced with lin-28, suggesting that Lin-28 plays an important role to improve the efficiency of establishment of iPS cells.

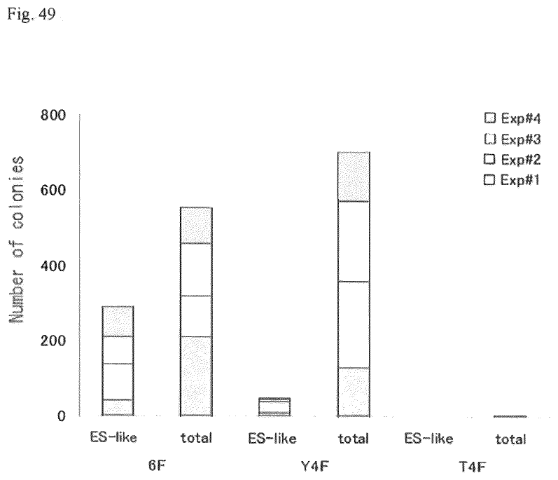

In addition, iPS cell induction experiments were performed with six factors (Klf4, c-Myc, Oct3/4, Sox2, Nanog, and Lin28) and two different combinations of four factors (Klf4, c-Myc, Oct3/4 and Sox2, referred to as Y4F in FIG. 49; and Oct3/4, Sox2, Nanog, and Lin-28, referred to as T4F in FIG. 49). The second combination of four factors, T4F, is the same combination as disclosed in Yu et al. Science 318:1917-1920,

2007. In these experiments, use of the six factors and the Y4F combination of four factors generated colonies having a similar morphology as ES-like cell colony, whereas the T4F combination generated no colonies having a similar morphology as ES-like cell colony (FIG. 49).

Example 23: Increased Efficiency of iPS Cell Generation with Sall4.

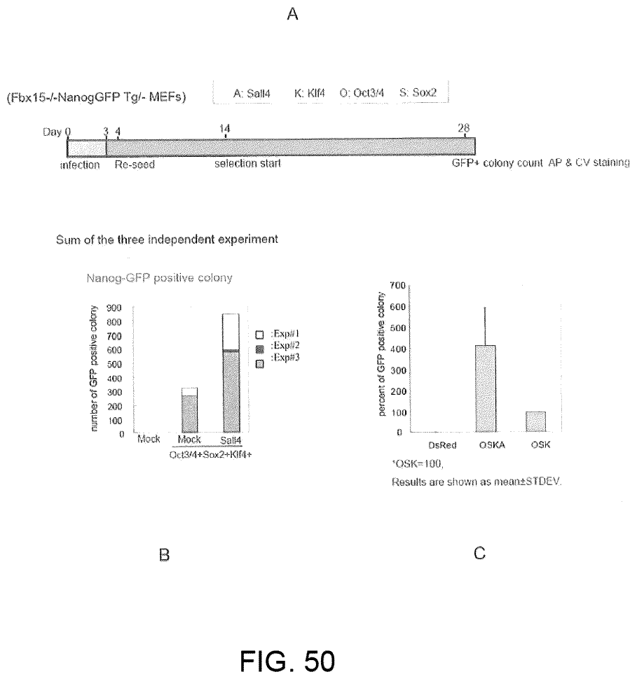

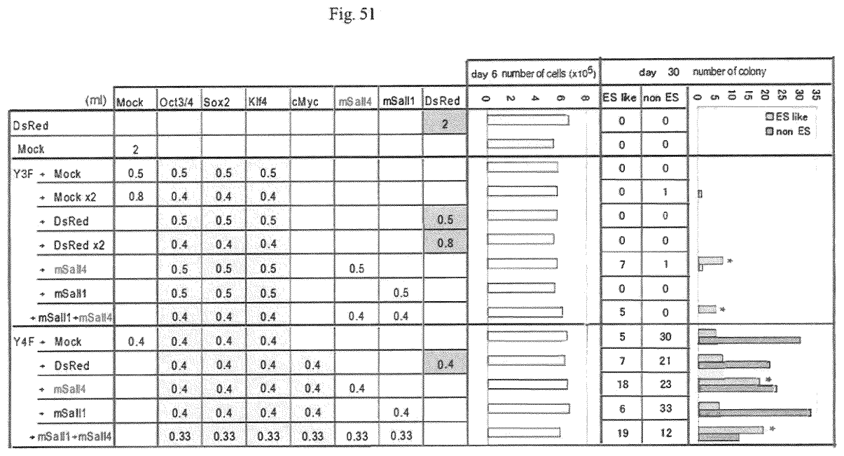

Experiments performed with mouse embryonic fibroblasts and adult human dermal fibroblasts showed that iPS cell induction with three factors (Klf4, Oct3/4, and Sox2) is more efficient when Sall4 is added to the combination, that is when Klf4, Oct3/4, Sox2, and Sall4 are used (FIGS. 50A-C and FIG. 51). More ES like colonies were also observed when Sal14 was added to the four factors (Klf4, Oct3/4, so-x2, and c-Myc) or the three factors (Klf4, Oct3/4, Sox2) under the experimental conditions used. These experiments show that addition of Sall4 to the nuclear reprogramming factor can improve iPS induction efficiency.

Example 24: Improved iPS Cell Generation with Mouse Sall4 and/or Mouse Sall1

In accordance with the method described in Takahashi et al. Cell 131:861-872, 2007, 4 factors (Y4F, i.e., OCT3/4, SOX2, KLF4, c-MYC) or 3 factors (Y3F, i.e., OCT3/4, SOX2, KLF4) derived from human, along with mouse Sall4 (mSall4) and/or mouse Sall1 (mSall1) were transduced with retroviruses into adult human dermal fibroblasts (HDFs) expressing mouse ecotropic receptor Slc7a, which was transduced with lentivirus.

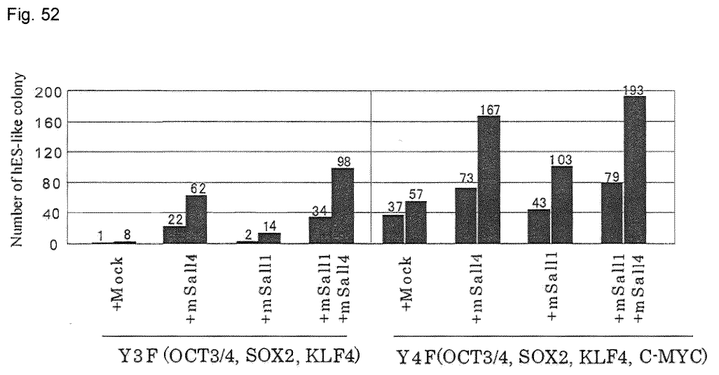

On the sixth day after the transduction, HDFs were harvested once. Then, 5×10⁶ HDFs were plated on 1.5×10⁶ STO cells treated with mitomycin C. From the next day, HDFs were cultured with the medium for primate ES cells (ReproCELL) containing 4 ng/ml recombinant human bFGF (WAKO). FIG. 52 shows the number of ES-like colonies at the 32^{nd} day and the 40^{th} day after the infection.

On the 32^{nd} day after the infection, the number of colonies in the case where Y3F and Mock were transduced was 1, and the number of colonies in the case where Y4F and Mock were transduced was 37. Whereas, the number of colonies in the case where Y3F and mSall4 were transduced was 22 on the 32^{nd} day after infection, and the number of colonies in the case where Y4F and mSall4 were transduced was 73 on the 32^{nd} day after infection.

In the case where Y3F and mSall1 were transduced, the number of colonies was 2 on the 32^{nd} Day after infection, and the number of colonies in the case where Y4F and mSall1 were transduced was 43 on the 32^{nd} day after infection. Where mSall1 was transduced together with Y3F or Y4F, the number of colonies on the 32^{nd} day after infection was about the same as when only Y3F or Y4F was transduced. Furthermore, in the case where both mSall4 and mSall1 together with Y3F were transduced, the number of colonies was 34 on the 32^{nd} day after infection; whereas in the case where both mSall4 and mSall1 together with Y4F were transduced, the number of colonies was 79 on the 32^{nd} day after infection.

On the 40^{th} day after the infection, the number of colonies in the case where Y3F and Mock were transduced was 8, and the number of colonies in the case where Y4F and Mock were transduced was 57. Whereas, in the case where mSall4 together with Y3F were transduced, the number of colonies was 62 on the 40^{th} day after infection, and the number of colonies in the case where Y4F and mSall4 were transduced was 167 on the 40^{th} day after infection. In the case where m Sall 1 together with Y3F were transduced, the number of colonies was 14 on the 40^{th} day after infection, and the number of colonies in the case where Y4F together with mSall1 were transduced was 103 on the 40^{th} day, after infection. Furthermore, in the case where Y3F, mSall4 and niSall1 were transduced together, the number of colonies on the 40^{th} day after infection was 98, while the number of colonies on the 40^{th} day after infection in the case where Y4F, mSall4 and mSall1 were transduced was 193.

As shown by the above, human ES-like colonies were obtained 20 to 100 times more efficiently in the case where mSall4 or both mSall4 and mSall1, together with Y3F, were transduced. In addition, human ES-like colonies were obtained 2 to 4 times more efficiently in the case where mSall4 or both mSall4 and mSall1, together with Y4F, were transduced.

Example 25: Improved iPS Cell Generation with Mouse Sall4 and/or Mouse Sall 1

In accordance with the method described in Takahashi et al. Cell 131:861-872, 2007, 4 factors (T4F, i.e., OCT3/4, SOX2, NANOG, LIN-28) or 3 factors (T3F, i.e., OCT3/4, SOX2, NANOG) derived from human, along with and mouse Sall4 (mSall4), mouse Sall1 (mSall1) or both mSall4 and mSall1 were transduced with retroviruses into adult human dermal fibroblasts (HDFs) expressing mouse ecotropic receptor Slc7a, which was transduced with lentivirus.

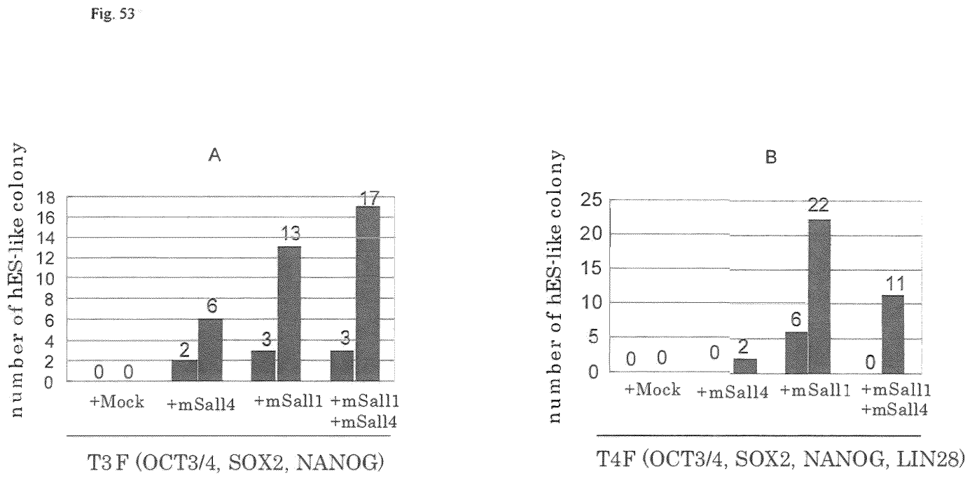

On the sixth day after the introduction, HDFs were harvested once. Then, 5×10⁵ HDFs were plated on 1.5×10⁶ STO cells treated with mitomycin C. On the seventh day after the incubation, HDFs were cultured with the medium for primate ES cells (ReproCELL) containing 4 ng/ml recombinant human bFGF (WAKO). FIGS. 53A-B show the number of ES-like colonies at the 32^{nd} day and the 40^{th} day after the infection.

On the 32^{nd} day after the infection, the number of colonies in the case where T3F and Mock or T4F and Mock were transduced was 0. In the case where mSall4 together with T3F were transduced, the number of colonies was 2 on the 32^{nd} day after infection. However, on the 32^{nd} day after infection no ES-like colony could be confirmed in the case where T4F and mSall4 were transduced.

In the case where mSall1 together with T3F were transduced, the number of colonies was 3 on the 32^{nd} day after infection, and the number of colonies in the case where T4F and mSall1 were transduced was 6 on the 32^{nd} day after infection. Furthermore, in the case where both mSall4 and mSall1 together with T3F were transduced, the number of colonies was 3 on the 32^{nd} day after infection. However, on the 32^{nd} day after infection no ES-like colony could be confirmed in the case where T4F, mSall4 and mSall1 were transduced together.

On the 40^{th} day after the infection, the number of colonies in the case where T3F and Mock or T4F and Mock were transduced was 0. Whereas, in the case where mSall4 together with T3F were transduced, the number of colonies on the 40^{th} day after infection was 6, and the number of colonies on the 40^{th} day after infection in the case where T4F and mSall4 were transduced was 2.

In the case where mSall1 together with T3F were transduced, the number of colonies on the 40^{th} day after infection was 13, and the number of colonies in the case where T4F and mSall1 were transduced together was 22 on the 40^{th} day after infection. Furthermore, in the case where both mSall4 and mSall1 together with T3F were transduced, the number of colonies on the 40^{th} day after infection was 17. In the case where both mSall4 and mSall1 together with T4F were transduced, the number of colonies on the 40^{th} day after infection was 11.

In the case where T3F or T4F were transduced to establish iPS cells, it was observed that the addition of mSall4 had an improvement effect on the formation of human ES-like cell colonies. Furthermore, the addition of mSall1 improved the effect on the formation of human ES-like cell colonies more efficiently than mSall4. As shown by the above, the addition of mSall1 greatly enhanced the efficiency of human ES-like cell colony formation, and had a greater improvement effect on the formation of human ES-like cell colonies when used in combination with T3F or T4F than the addition of mSall4 did.

Furthermore, it was shown that the efficiency of human ES-like cell colony formation was much improved in the case where both mSall4 and mSall1 together with T3F or T4F were transduced.

Example 26: Improved iPS Cell Generation with Human Sall4

In accordance with the method described in Takahashi et al. Cel/131:861-872, 2007, adult human dermal fibroblasts (HDFs) expressing mouse ecotropic receptor Slc7a which was transduced with lentivirus were prepared. On the next day, 4 factors (Y4F, i.e., OCT3/4, SOX2, KLF4, c-MYC) or 3 factors (Y3F, i.e., OCT3/4, SOX2, KLF4) derived from human, and human SALL4 (hSALL4) were transduced with retroviruses into HDFs.

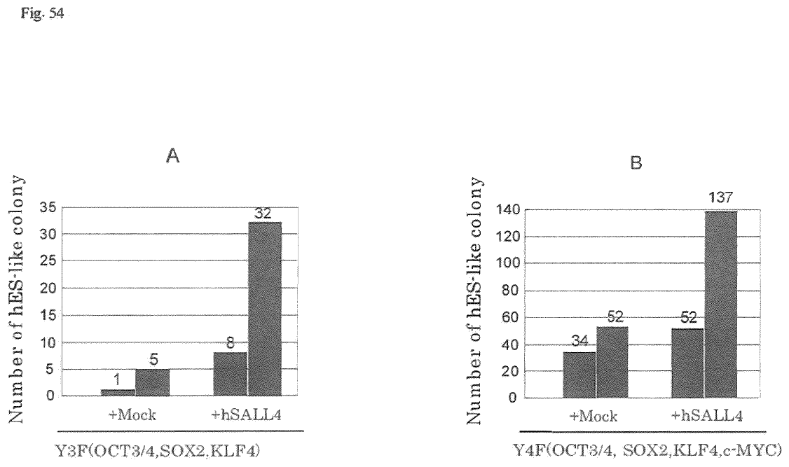

On the sixth day after the transduction, HDFs were harvested once. Then, 5×10⁵ HDF cells were plated on 1.5×10⁶ STO cells treated with mitomycin C. From the next day, HDFs were cultured with the medium for primate ES cells (ReproCELL) containing 4 ng/ml recombinant human bFGF (WAKO). FIGS. 54A-B show the number of ES-like colonies at the 32^{nd} day and the 40^{th} day after the infection.

On the 32^{nd} day after the infection, the number of colonies in the case where Y3F and Mock were transduced was 1, and the number of colonies in the case where Y4F and Mock were transduced was 34. Whereas, in the case where hSALL4 together with Y3F were transduced, the number of colonies was 8 on the 32^{nd} day after infection, and the number of colonies in the case where Y4F and hSALL4 were transduced was 52 on the 32^{nd} day after infection.

On the 40^{th} day after the infection, the number of colonies in the case where Y3F and Mock were transduced was 5, and the number of colonies in the case where Y4F and Mock were transduced was 52. Whereas, in the case where hSALL4 together with Y3F were transduced, the number of colonies was 32 on the 40^{th} day after infection, and the number of colonies in the case where Y4F and hSALL4 were transduced was 137 on the 40^{th} day after infection.

As shown by the above, human ES-like colonies were obtained 6 to 8 times more efficiently in the case where hSALL4 together with Y3F were transduced. In addition, the formation of human ES-like colonies was improved 1.5 to 2.5 times more efficiently in the case where hSALL4 together with Y4F were transduced.

### Example 27: Effect of L-Myc

### (A) Nuclear Reprogramming by Transduction of 4 Genes (Oct3/4, K/f4. Sox2, and L-Myc)

### (1) Effect on Establishment of Human iPS Cells

Adult human dermal fibroblasts (aHDF-Slc7a1) expressing mouse ecotropic virus receptor (Slc7a1) were prepared in accordance with the method described in Takahashi et al. Cell 131:861-872, 2007. The aHDF-Slc7a1 cells were plated at 3×10⁵ cells/60 mm dish. On the next day, 4 genes derived from human (OCT3/4, KLF4 and SOX2 plus either L-MYC1 (hereinafter referred to as L-MYC), c-MYC or N-MYC) were transduced by retrovirus in accordance with the method described in Takahashi et al. Cell 131:861-872, 2007. On the sixth day after virus infection, the cells were recovered, and were plated again on MSTO cells at 5×10⁵ cells/100 mm dish. From the next date, the cells were cultured in a medium obtained by adding 4 ng/ml recombinant human bFGF (WAKO) to the medium for primate ES cells (ReproCELL). On the 37^{th} day after retrovirus infection, the number of human iPS colonies (ES-like colonies) which were appearing was counted. TABLE 18 and FIG. 55 show the results of four experiments. FIG. 55 is a graph of the results of TABLE 18. The values on the graph show the ratio of the iPS colony number to the total colony number.

TABLE 18
< Number of hiPS colonies >

| | | ES-like | Total | % |
|---|---|---|---|---|
| h32 | c-MYC | 3 | 38 | 7.89 |
| ▪ | L-MYC | 18 | 35 | 51.4 |
| | N-MYC | 32 | 431 | 7.42 |
| h44 | c-MYC | 11 | 14 | 78.6 |
| ▪ | L-MYC | 82 | 100 | 82.0 |
| | N-MYC | 31 | 63 | 49.2 |
| h51 | c-MYC | 7 | 69 | 10.1 |
| ▭ | L-MYC | 62 | 124 | 50.0 |
| | N-MYC | 25 | 225 | 11.1 |
| h58 | c-MYC | 14 | 55 | 25.5 |
| ▭ | L-MYC | 53 | 70 | 75.7 |
| | N-MYC | 52 | 298 | 17.4 |

As shown in TABLE 18 and FIG. 55, the efficacy of establishment of iPS colonies was increased 6 times by using L-MYC, as compared with the case of using c-Myc. Also, the ratio of the iPS colony (ES-like colony) number to the total colony number was increased 2 times by using L-MYC, as compared with the case of using c-MYC. Further, the efficacy of establishment of iPS colony was increased by using N-MYC as compared with the case of using c-MYC, although the increase of the efficacy was lower than that in the case of using L-MYC.

It has been reported that there is no difference in efficacy between c-MYC and L-MYC in the establishment of iPS cells derived from mouse fibroplast cells (MEF) (Nakagawa et al. Nature Biotechnology 26 (1): 101-106, 2008). As mentioned above, it has been revealed that the efficacy of establishment of human iPS cells is remarkably increased by using L-MYC in place of c-MYC.

### (2) In Vitro Differentiation Induction

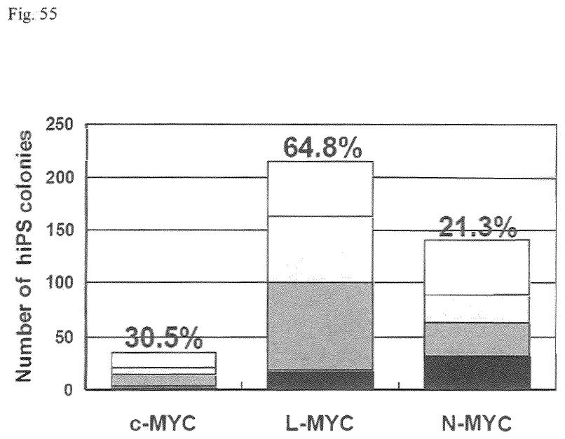

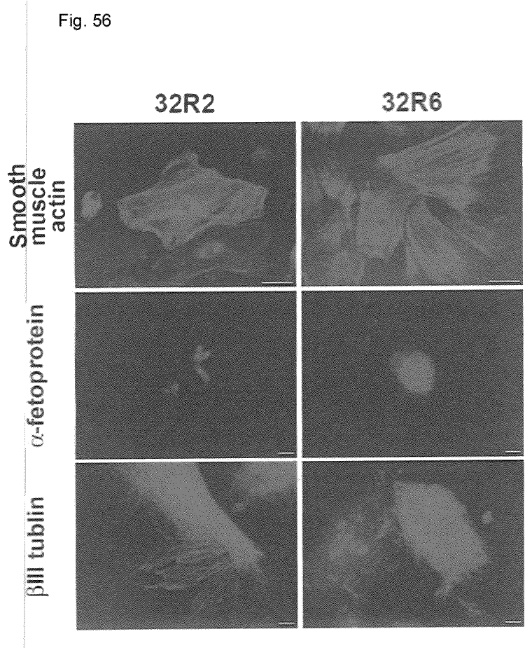

Human iPS cells (32R2, 32R6) which were established by transduction of 4 genes (OCT3/4, KLF4, SOX2, and L-MYC) were plated on a low-binding dish, and embryoid bodies (EB) were formed on 100 mm dish in accordance with the method described in Takahashi et al. Cell 131:861-872, 2007. After culturing for 2 weeks, the cells were stained using an antibody against each of α-fetoprotein (R&D systems) which is a differentiation marker for endodermal cells, smooth muscle actin (DAKO) which is a differentiation marker for mesodermal cells, and βIII-tubulin (Chemicon) which is a differentiation marker for ectodermal cells. The results are shown in FIG. 56. The expression of each marker was confirmed by staining. It was confirmed that the established human iPS cells have an ability to differentiate into three germ layers.

### (3) Teratoma Forming Ability

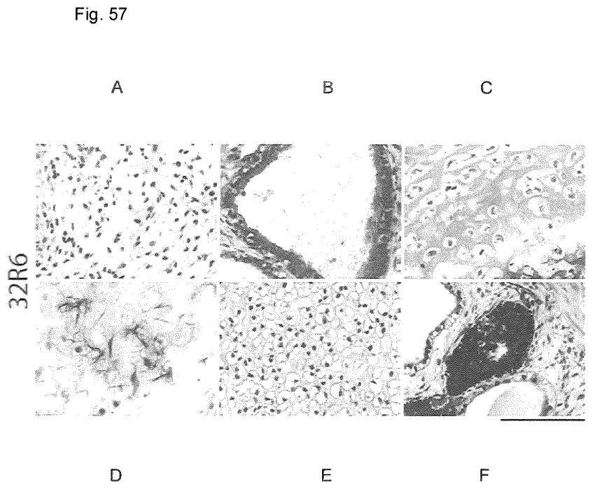

Human iPS cells (32R6) which were established by transduction of 4 genes (OCT3/4, KLF4, SOX2, and L-MYC) were cultured in the medium for primate ES cells (ReproCELL) containing recombinant human bFGF (4 ng/ml) and Rho kinase inhibitor Y-27632 (10 μM). After 1 hour, the cells were collected by treatment with collagen IV. Then, the cells were centrifuged and collected, and were suspended in DMEM/F12 containing Y-27632 (10 μM). A quarter of the confluent cells on 100 mm dish was injected into testis of SCID mouse. After two to three months, the tumor was cut into pieces and was fixed in PBS(−) containing 4% formaldehyde. Paraffin-embedded tissue was sliced and was stained with hematoxylin-eosin. The results are shown in FIGS. 57A-F. Histogenetically, the tumor is composed of several types of cells. Nerve tissues, bowel-like tissue, cartilage tissue, hair tissue, adipose cell and pigment tissue were observed. Thus, pluripotential ability of the iPS cell was demonstrated.

### (4) Effect on Tumorigenesis of Chimera Mouse

MEFs (MEF-Ng) were isolated from a mouse having Nanog reporter which was prepared by inserting EGFP gene and puromycin-resistance gene into Nanog locus (Okita et al. Nature 448:313-17, 2007). Similarly, MEFs (MEF-Fbx) were isolated from a mouse having Fbx15 reporter which was prepared by inserting βgeo gene into Fbx15 locus (Takahashi et al. Cell 126:663-676, 2006). Four genes (Oct3/4, Klf4, Sox2, and L-Myc) were transduced into these MEFs by retrovirus. MEF-Ng was selected by puromycin, and MEF-Fbx was selected by G418. Thus, iPS cells, each of which is referred to as Nanog-iPS and Fbx-iPS, were established. These iPS cells were transplanted into blastocyst of C57BL/6 mouse to create a chimera mouse. The tumorigenesis in the chimera mouse and F1 mouse was evaluated. As a result, the mice are currently surviving for the period shown in TABLE 19, below, and no tumorgenesis was observed in any surviving mice.

TABLE 19
Summary of Tumorigenesis in Chimera Mice

| | Surviving Mice/Total | Days |
|---|---|---|
| Mouse Type | Number of Mice | Surviving |
| Chimera Fbx-iPS | 19/19 | 349 |
| Chimera Nanog-iPS | 15/16 | 363 |
| F1 Nanog-iPS | 1/1 | 255 |
| F1 Nanog-iPS | 3/3 | 181 |
| F1 Nanog-iPS | 1/1 | 132 |
| Chimera Nanog-iPS | 30/30 | 63 |
| Chimera Nanog-iPS | 12/14 | 62 |

In case of using iPS cell which was obtained by reprogramming with 4 genes (Oct3/4, Klf4, Sox2, and c-Myc), 6 chimera mice among 37 mice died due to tumor within 100 days after birth (Nakagawa et al. Nature Biotechnology 26(1): 101-106, 2008). On the other hand, it was revealed that, when L-Myc was used in place of c-Myc as mentioned above, the survival time was increased and the tumorigenesis was significantly decreased.

### (B) Nuclear Reprogramming by Transduction of 3 Genes (Oct3/4, Klf4, and L-Myc)

### (1) Formation of iPS Colony

Establishment of iPS cells was attempted by transduction of 3 genes (Oct3/4, Klf4, L-Myc) in accordance with the method described in Nakagawa et al. Nature Biotechnology 26 (1): 101-106, 2008. The MEF-Ng described in (A)(4) above were plated on gelatin-coated 6-well culture plates at 1.3×10⁵ cells/well. 24 hours later, three mouse-derived genes (Oct3/4, Klf4, and L-Myc) were transduced by retrovirus.

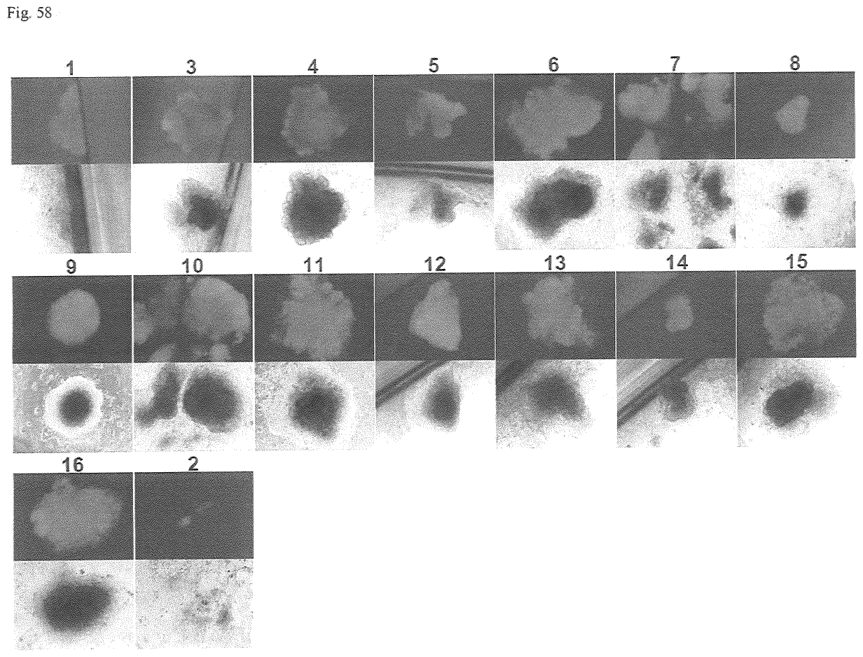

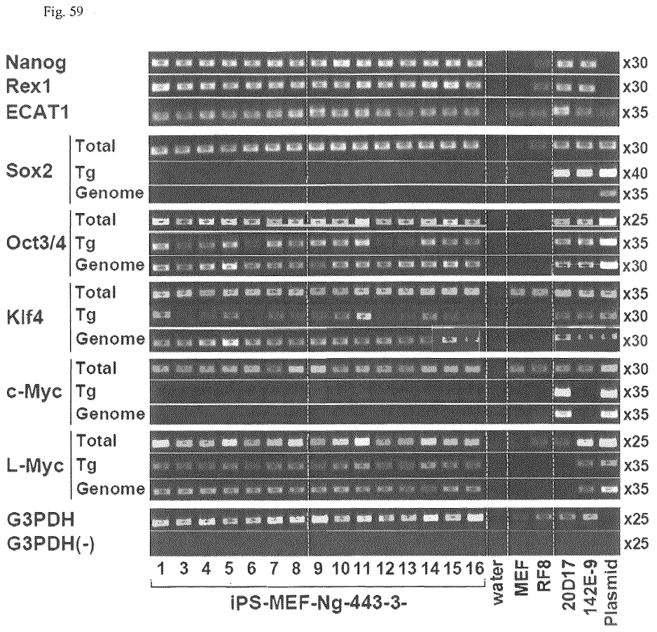

On the fourth day after virus infection, the cells were recovered, and plated again on MSTO cells at 1.5×10⁵ cells/6-well plate. From the next date, the cells were cultured in a culture medium for ES cells (which was obtained by adding, to DMEM (Nacarai tesque), 15% fetal bovine serum, 2 mM L-glutamine (Invitrogen), 100 μM non-essential amino acids (Invitrogen), 100 μM 2-mercaptoethanol (Invitrogen), 50 U/mL penicillin (Invitrogen) and 50 mg/mL streptomycin (Invitrogen)) supplemented with LIF. On the 46^{th} day after virus infection, GFP positive colonies were picked up (iPS-MEF-Ng-443-3 clone). The photograph of the 16 picked-up cells is shown in FIG. 58. All the cells were GFP-positive iPS colonies. Using these GFP-positive iPS colony-derived clones, RT-PCR and genomic PCR were carried out to analyze the gene expression. The results are shown in FIG. 59. Nanog, Rex1 and ECAT1, which are marker genes for ES cells, were expressed in all the clones. Also, the three transduced genes (Oct3/4, Klf4, L-Myc) were integrated into the genome in all of the iPS colonies which were examined. In some clones, the expression of mRNA was not detected, possibly due to silencing.

### (2) In Vitro Differentiation Induction

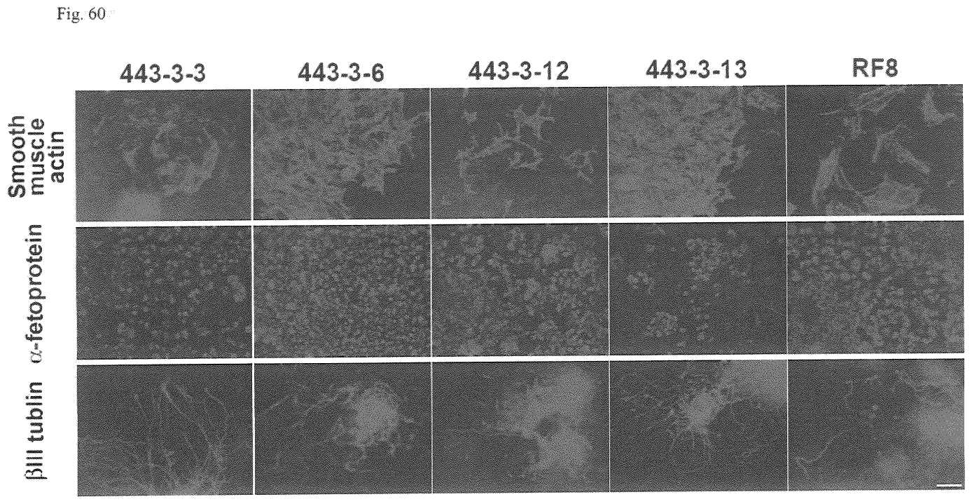

The four clones (iPS-MEF-Ng-443-3-3, iPS-MEF-Ng-443-3-6, iPS-MEF-Ng-443-3-12, and iPS-MEF-Ng-443-3-13) which were established in (1) above, were plated on a low-binding dish at 2×10⁶ cells/6-well plate to form embryoid bodies (EB). The cells were cultured for 2 weeks in the culture medium for ES cells, and were then stained using an antibody against each of α-fetoprotein (R&D systems) which is a differentiation marker for endodermal cells, smooth muscle actin (DAKO) which is a differentiation marker for mesodermal cells, and βIII-tubulin (Chemicon) which is a differentiation marker for ectodermal cells. The results are shown in FIG. 60. The expression of each marker was confirmed by staining. It was confirmed that the established mouse iPS cells have an ability to differentiate into three germ layers.

### (3) Teratoma Forming Ability

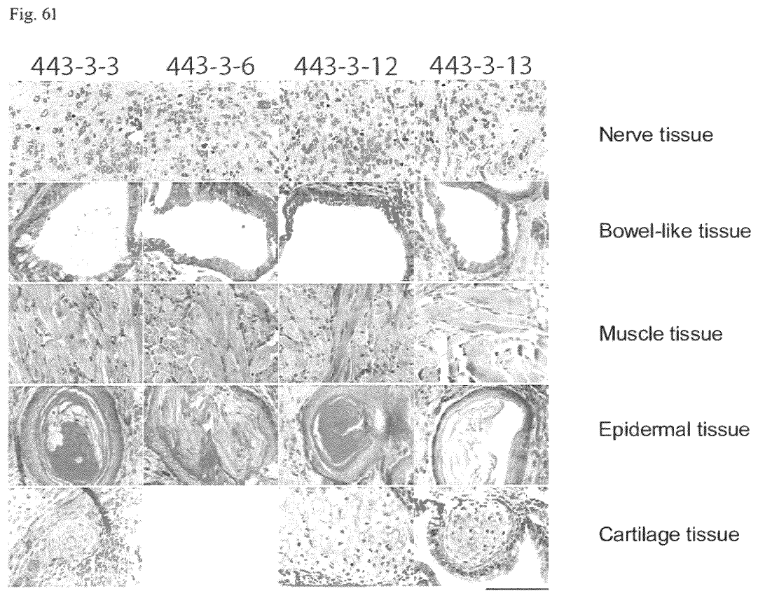

The four clones (iPS-MEF-Ng-443-3-3, iPS-MEF-Ng-443-3-6, iPS-MEFNg-443-3-12, iPS-MEF-Ng-443-3-13) which were established in (1) above, were subcutaneously injected into a nude mouse, respectively. After 4 weeks, teratoma formation was observed in all the cases. Histogenetically, the tumor is composed of several types of cells. Nerve tissues, bowel-like tissue, muscle tissue, epidermal tissue and cartilage tissue were observed (FIG. 61). Thus, pluripotential ability of the iPS cell was demonstrated.

### Example 28: Effect of L-MYC and LIN28 on Induction of Human iPS Cells

Adult human dermal fibroblasts (aHDF-Slc7a1) expressing mouse ecotropic virus receptor (Slc7a1) were prepared in accordance with the method described in Takahashi et al. Cell 131:861-872, 2007. The aHDF-Slc7a1 cells were plated at 3×10⁵ cells/60 mm dish. On the next day, the following human-derived 3 genes, 4 genes or 5 genes were transduced by retrovirus in accordance with the method described in Takahashi et al. Cell 131:861-872, 2007.
1. SOX2, OCT3/4, KLF4
2. SOX2, OCT3/4, KLF4, c-MYC
3. SOX2, OCT3/4, KLF4, L-MYC
4. SOX2, OCT3/4, KLF4, LIN28
5. SOX2, OCT3/4, KLF4, c-MYC, LIN28
6. SOX2, OCT3/4, KLF4, L-MYC, LIN28.

On the sixth day after virus infection, the cells were recovered, and were plated again on MSTO cells at 5×10⁵ cells/100 mm dish. From the next date, the cells were cultured in a medium obtained by, adding 4 ng/ml recombinant human bFGF (WAKO) to a medium for primate ES cells (ReproCELL).

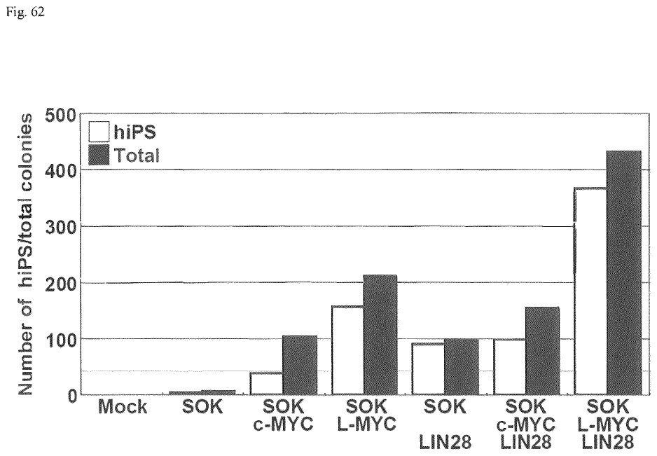

On the 37^{th} day after retrovirus infection, the numbers of the total colonies which appeared and the number of human iPS colonies (hiPS colony) were counted. The results are shown in TABLE 20 and FIG. 62. FIG. 62 is a graph of the result of TABLE 20.

TABLE 20
Summary of hiPS Cell Induction Results with L-MYC and

| | | | | | | | hiPS | Total | hiPS |
|---|---|---|---|---|---|---|---|---|---|
| | | | | | | | colonies | colonies | (%) |
| 1 | Mock | DsRed | Mock | Mock | Mock | A | 0 | 0 | 0 |
| 2 | SOX2 | OCT4 | KLF4 | Mock | Mock | D | 3 | 5 | 60.0 |
| 3 | SOX2 | OCT4 | KLF4 | c-MYC | Mock | B | 38 | 103 | 36.9 |
| 4 | SOX2 | OCT4 | KLF4 | L-MYC | Mock | C | 156 | 211 | 73.9 |
| 5 | SOX2 | OCT4 | KLF4 | Mock | LIN28 | G | 90 | 97 | 92.8 |
| 6 | SOX2 | OCT4 | KLF4 | c-MYC | LIN28 | E | 98 | 154 | 63.6 |
| 7 | SOX2 | OCT4 | KLF4 | L-MYC | LIN28 | F | 366 | 432 | 84.7 |

By adding LIN28 to the 4 genes (SOX2, OCT3/4, KLF4 and L-MYC), the number of iPS colonies was significantly increased. The number of iPS colonies was much higher than the number of colonies in the case of adding LIN28 to the 4 genes (SOX2, OCT3/4, KLF4 and c-MYC). Further, LIN28 showed a synergistic effect when used in combination with the above-described 4 genes which included c-MYC, and showed an even stronger synergistic effect when used in combination with the above-described 4 genes which included L-MYC (TABLE 20). Further, the ratio of the iPS colony number to the total colony number was 84.7% which is a very high value, when five genes (SOX2, OCT3/4, KLF4, L-MYC, and LIN28) were used. From the aforementioned result, it was revealed that a combination of 5 genes (SOX2, OCT3/4, KLF4, L-MYC, and LIN28) was extremely effective for increasing the efficacy of establishment of human iPS cells.

### Example 29: Effect of Chimeric and Mutant Myc Genes on Induction of Human iPS Cells

Various Myc chimera genes (Ms-cL-Myc, Ms-Lc-Myc, Ms-cLc-Myc, Ms-LcL-Myc, Ms-ccL-Myc, Ms-cLL-Myc, Ms-LLc-Myc, and Ms-Lcc-Myc) and Myc point-mutated genes (c-Myc-W135E, c-Myc-V394D, c-Myc-L420P, L-Myc-W96E, L-Myc-V325D and L-Myc-L351P) were constructed. Regarding the Myc point-mutants, c-Myc-W135E comprises an amino acid substitution within a domain essential for interaction with a nuclear factor that is required for E1 A-mediated cell transformation (Brough et al. Molecular and Cellular Biology 15(3): 1536-1544, 1995, which is herein incorporated by reference in its entirety). Another Myc point-mutant, c-Myc-L420P, loses its ability to bind to MAX protein (Blackwood et al. Science 251(4998): 1211-17, 1991, which is herein incorporated by reference in its entirety). Yet another Myc point-mutant, c-Myc V394D, loses its ability to bind to MIZ-1 protein (Herold et al. Mol. Cell 10(3): 509-21, 2002, which is herein incorporated by reference in its entirety). L-Myc-W96E, L-Myc-V325D and L-Myc-L351P are corresponding point mutants of c-Myc-W135E, c-Myc-V394D, and c-Myc-L420P.

TABLE 21 discloses the amino acid and nucleic acid sequences of the Myc constructs:

TABLE 21
Myc, Myc Chimera, and Myc Variant Amino Acid and
Nucleotide Sequences

| | |
|---|---|
| Ms-c-Myc | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMWSGFSAAAKLVSEKLASY |
| | QAARKDSTSLSPARGHSVCSTSSLYLQDLTAAASECIDPSVVFPYPLNDSS |
| | SPKSCTSSDSTAFSPSSDSLLSSESSPRASPEPLVLHEETPPTTSSDSEEE |
| | QEDEEEIDVVSVEKRQTPAKRSESGSSPSRGHSKPPHSPLVLKRCHVSTHQ |
| | HNYAAPPSTRKDYPAAKRAKLDSGRVLKQISNNRKCSSPRSSDTEENDKRR |
| | THNVLERQRRNELKRSFFALRDQIPELENNEKAPKVVILKKATAYILSIQA |
| | DEHKLTSEKDLLRKRREQLKHKLEQLRNSGA (SEQ ID NO: 134) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGTGG |
| | AGCGGTTTCTCAGCCGCTGCCAAGCTGGTCTCGGAGAAGCTGGCCTCCTAC |
| | CAGGCTGCGCGCAAAGACAGCACCAGCCTGAGCCCCGCCCGCGGGCACAGC |
| | GTCTGCTCCACCTCCAGCCTGTACCTGCAGGACCTCACCGCCGCCGCGTCC |
| | GAGTGCATTGACCCCTCAGTGGTCTTTCCCTACCCGCTCAACGACAGCAGC |
| | TCGCCCAAATCCTGTACCTCGTCCGATTCCACGGCCTTCTCTCCTTCCTCG |
| | GACTCGCTGCTGTCCTCCGAGTCCTCCCCACGGGCCAGCCCTGAGCCCCTA |
| | GTGCTGCATGAGGAGACACCGCCCACCACCAGCAGCGACTCTGAAGAAGAG |
| | CAAGAAGATGAGGAAGAAATTGATGTGGTGTCTGTGGAGAAGAGGCAAACC |
| | CCTGCCAAGAGGTCGGAGTCGGGCTCATCTCCATCCCGAGGCCACAGCAAA |
| | CCTCCGCACAGCCCACTGGTCCTCAAGAGGTGCCACGTCTCCACTCACCAG |
| | CACAACTACGCCGCACCCCCCTCCACAAGGAAGGACTATCCAGCTGCCAAG |
| | AGGGCCAAGTTGGACAGTGGCAGGGTCCTGAAGCAGATCAGCAACAACCGC |
| | AAGT (SEQ ID NO: 135) |
| Ms-L-Myc | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMWSGFSAR |
| | ERLERVVSDRLAPGAPRGNPPKAPATPDGTPSLEASNPAPATQCQLGEPKT |
| | QACSGSESPSDSEGEEIDVVTVEKRRSLDIRKPVTITVRADPLDPCMKHFH |
| | ISIHQQQHNYAARFPPESCSQEGDPEPGPQEEAPEIEAPKEKEEEEEEEEE |
| | EEIVSPPPVGSEAPQSCHPKPVSSDTEDVTKRKNHNFLERKRRNDLRSRFL |
| | ALRDQVPTLASCSKAPKVVILSKALEYLQALVGAEKKMATEKRQLRCRQQQ |
| | LQKRIAYLSGY (SEQ ID NO: 136) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGTGGAGCGGCTTCTCCGCCCGA |
| | GAACGGCTGGAGAGAGTGGTGAGCGACAGGCTGGCCCCAGGCGCGCCCCGG |
| | GGGAACCCGCCCAAAGCGCCCGCTACCCCGGACGGCACTCCTAGTCTGGAA |
| | GCCAGTAACCCGGCGCCCGCCACCCAATGTCAGCTGGGCGAGCCCAAGACT |
| | CAGGCCTGCTCCGGGTCCGAGAGCCCCAGCGATTCTGAAGGTGAAGAGATT |
| | GACGTGGTGACCGTGGAGAAGAGGCGATCTCTGGACATCCGAAAGCCAGTC |
| | ACCATCACGGTGCGAGCAGACCCCCTGGACCCCTGCATGAAGCACTTCCAT |
| | ATCTCTATCCACCAACAGCAGCATAACTATGCTGCCCGTTTTCCTCCAGAA |
| | AGTTGCTCTCAAGAGGGGGATCCTGAGCCAGGTCCCCAGGAAGAGGCTCCG |
| | GAGATAGAAGCTCCCAAGGAGAAAGAGGAGGAGGAAGAGGAAGAGGAGGAA |
| | GAAGAGATTGTGAGCCCCCCACCTGTCGGAAGTGAGGCTCCCCAGTCCTGC |
| | CACCCCAAACCTGTCAGTTCTGACACTGAGGACGTGACCAAGAGGAAGAAC |
| | CATAACTTCTTGGAACGAAAAAGGAGGAATGACCTCCGCTCCCGGTTCCTA |
| | GCCCTGCGGGACCAGGTTCCCACCCTGGCCAGCTGCTCTAAGGCCCCCAAA |
| | GTCGTGATCCTCAGCAAGGCGTTAGAATACTTGCAGGCTTTGGTGGGGGCT |
| | GAAA (SEQ ID NO: 137) |
| Ms-cL-Myc | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMWSGFSAAAKLVSEKLASY |
| | QAARKDSTSLSPARGHSVCSTSSLYLQDLTAAASECIDPSVVFPYPLNDSS |
| | SPKSCTSSDSTAFSSDSEGEEIDVVTVEKRRSLDIRKPVTITVRADPLDPC |
| | MKHFHISIHQQQHNYAARFPPESCSQEGDPEPGPQEEAPEIEAPKEKEEEE |
| | EEEEEEEIVSPPPVGSEAPQSCHPKPVSSDTEDVTKRKNHNFLERKRRNDL |
| | RSRFLALRDQVPTLASCSKAPKVVILSKALEYLQALVGAEKKMATEKRQLR |
| | CRQQQLQKRIAYLSGY (SEQ ID NO: 138) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGTGG |
| | AGCGGTTTCTCAGCCGCTGCCAAGCTGGTCTCGGAGAAGCTGGCCTCCTAC |
| | CAGGCTGCGCGCAAAGACAGCACCAGCCTGAGCCCCGCCCGCGGGCACAGC |
| | GTCTGCTCCACCTCCAGCCTGTACCTGCAGGACCTCACCGCCGCCGCGTCC |
| | GAGTGCATTGACCCCTCAGTGGTCTTTCCCTACCCGCTCAACGACAGCAGC |
| | TCGCCCAAATCCTGTACCTCGTCCGATTCCACGGCCTTCTCTAGCGATTCT |
| | GAAGGTGAAGAGATTGACGTGGTGACCGTGGAGAAGAGGCGATCTCTGGAC |
| | ATCCGAAAGCCAGTCACCATCACGGTGCGAGCAGACCCCCTGGACCCCTGC |
| | ATGAAGCACTTCCATATCTCTATCCACCAACAGCAGCATAACTATGCTGCC |
| | CGTTTTCCTCCAGAAAGTTGCTCTCAAGAGGGGGATCCTGAGCCAGGTCCC |
| | CAGGAAGAGGCTCCGGAGATAGAAGCTCCCAAGGAGAAAGAGGAGGAGGAA |
| | GAGGAAGAGGAGGAAGAAGAGATTGTGAGCCCCCCACCTGTCGGAAGTGAG |
| | GCTCCCCAGTCCTGCCACCCCAAACCTGTCAGTTCTGACACTGAGGACGTG |
| | ACCA (SEQ ID NO: 139) |
| Ms-Lc-Myc | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMWSGFSAR |
| | ERLERVVSDRLAPGAPRGNPPKAPATPDGTPSLEASNPAPATQCQLGEPKT |
| | QACSGSESPSDSLLSSESSPRASPEPLVLHEETPPTTSSDSEEEQEDEEEI |
| | DVVSVEKRQTPAKRSESGSSPSRGHSKPPHSPLVLKRCHVSTHQHNYAAPP |
| | STRKDYPAAKRAKLDSGRVLKQISNNRKCSSPRSSDTEENDKRRTHNVLER |
| | QRRNELKRSFFALRDQIPELENNEKAPKVVILKKATAYILSIQADEHKLTS |
| | EKDLLRKRREQLKHKLEQLRNSGA (SEQ ID NO: 140) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGTGGAGCGGCTTCTCCGCCCGA |
| | GAACGGCTGGAGAGAGTGGTGAGCGACAGGCTGGCCCCAGGCGCGCCCCGG |
| | GGGAACCCGCCCAAAGCGCCCGCTACCCCGGACGGCACTCCTAGTCTGGAA |
| | GCCAGTAACCCGGCGCCCGCCACCCAATGTCAGCTGGGCGAGCCCAAGACT |
| | CAGGCCTGCTCCGGGTCCGAGAGCCCCTCGGACTCGCTGCTGTCCTCCGAG |
| | TCCTCCCCACGGGCCAGCCCTGAGCCCCTAGTGCTGCATGAGGAGACACCG |
| | CCCACCACCAGCAGCGACTCTGAAGAAGAGCAAGAAGATGAGGAAGAAATT |
| | GATGTGGTGTCTGTGGAGAAGAGGCAAACCCCTGCCAAGAGGTCGGAGTCG |
| | GGCTCATCTCCATCCCGAGGCCACAGCAAACCTCCGCACAGCCCACTGGTC |
| | CTCAAGAGGTGCCACGTCTCCACTCACCAGCACAACTACGCCGCACCCCCC |
| | TCCACAAGGAAGGACTATCCAGCTGCCAAGAGGGCCAAGTTGGACAGTGGC |
| | AGGGTCCTGAAGCAGATCAGCAACAACCGCAAGTGCTCCAGCCCCAGGTCC |
| | TCAGACACGGAGGAAAACGACAAGAGGCGGACACACAACGTCTTGGAACGT |
| | CAGAGGAGGAACGAGCTGAAGCGCAGCTTTTTTGCCCTGCGTGACCAGATC |
| | CCTGAATTGGAAAACAACGAAAAGGCCCCCAAGGTAGTGATCCTCAAAAAA |
| | GCCA (SEQ ID NO: 141) |
| Ms-cLc-Myc | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMWSGFSAAAKLERVVSDRL |
| | APGAPRGNPPKAPATPDGTPSLEASNPAPATQCQLGEPKTQACSGSESPSD |
| | SEGEEIDVVTVEKRRSLDIRKPVTITVRADPLDPCMKHFHISIHQQQHNYA |
| | ARFPPESCSQEGDPEPGPQEEAPEIEAPKEKEEEEEEEEEEEIVSPPPVGS |
| | EAPQSCHPKPVSSDTEENDKRRTHNVLERQRRNELKRSFFALRDQIPELEN |
| | NEKAPKVVILKKATAYILSIQADEHKLTSEKDLLRKRREQLKHKLEQLRNS |
| | GA (SEQ ID NO: 142) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGTGG |
| | AGCGGTTTCTCAGCCGCTGCCAAGCTGGAGAGAGTGGTGAGCGACAGGCTG |
| | GCCCCAGGCGCGCCCCGGGGGAACCCGCCCAAAGCGCCCGCTACCCCGGAC |
| | GGCACTCCTAGTCTGGAAGCCAGTAACCCGGCGCCCGCCACCCAATGTCAG |
| | CTGGGCGAGCCCAAGACTCAGGCCTGCTCCGGGTCCGAGAGCCCCAGCGAT |
| | TCTGAAGGTGAAGAGATTGACGTGGTGACCGTGGAGAAGAGGCGATCTCTG |
| | GACATCCGAAAGCCAGTCACCATCACGGTGCGAGCAGACCCCCTGGACCCC |
| | TGCATGAAGCACTTCCATATCTCTATCCACCAACAGCAGCATAACTATGCT |
| | GCCCGTTTTCCTCCAGAAAGTTGCTCTCAAGAGGGGGATCCTGAGCCAGGT |
| | CCCCAGGAAGAGGCTCCGGAGATAGAAGCTCCCAAGGAGAAAGAGGAGGAG |
| | GAAGAGGAAGAGGAGGAAGAAGAGATTGTGAGCCCCCCACCTGTCGGAAGT |
| | GAGGCTCCCCAGTCCTGCCACCCCAAACCTGTCTCCTCAGACACGGAGGAA |
| | AACGACAAGAGGCGGACACACAACGTCTTGGAACGTCAGAGGAGGAACGAG |
| | CTGA (SEQ ID NO: 143) |
| Ms-LcL-Myc | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMWSGFSAR |
| | ERLVSEKLASYQAARKDSTSLSPARGHSVCSTSSLYLQDLTAAASECIDPS |
| | VVFPYPLNDSSSPKSCTSSDSTAFSPSSDSLLSSESSPRASPEPLVLHEET |
| | PPTTSSDSEEEQEDEEEIDVVSVEKRQTPAKRSESGSSPSRGHSKPPHSPL |
| | VLKRCHVSTHQHNYAAPPSTRKDYPAAKRAKLDSGRVLKQISNNRKCSSPR |
| | SSDTEDVTKRKNHNFLERKRRNDLRSRFLALRDQVPTLASCSKAPKVVILS |
| | KALEYLQALVGAEKKMATEKRQLRCRQQQLQKRIAYLSGY (SEQ ID |
| | NO: 144) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGTGGAGCGGCTTCTCCGCCCGA |
| | GAACGGCTGGTCTCGGAGAAGCTGGCCTCCTACCAGGCTGCGCGCAAAGAC |
| | AGCACCAGCCTGAGCCCCGCCCGCGGGCACAGCGTCTGCTCCACCTCCAGC |
| | CTGTACCTGCAGGACCTCACCGCCGCCGCGTCCGAGTGCATTGACCCCTCA |
| | GTGGTCTTTCCCTACCCGCTCAACGACAGCAGCTCGCCCAAATCCTGTACC |
| | TCGTCCGATTCCACGGCCTTCTCTCCTTCCTCGGACTCGCTGCTGTCCTCC |
| | GAGTCCTCCCCACGGGCCAGCCCTGAGCCCCTAGTGCTGCATGAGGAGACA |
| | CCGCCCACCACCAGCAGCGACTCTGAAGAAGAGCAAGAAGATGAGGAAGAA |
| | ATTGATGTGGTGTCTGTGGAGAAGAGGCAAACCCCTGCCAAGAGGTCGGAG |
| | TCGGGCTCATCTCCATCCCGAGGCCACAGCAAACCTCCGCACAGCCCACTG |
| | GTCCTCAAGAGGTGCCACGTCTCCACTCACCAGCACAACTACGCCGCACCC |
| | CCCTCCACAAGGAAGGACTATCCAGCTGCCAAGAGGGCCAAGTTGGACAGT |
| | GGCAGGGTCCTGAAGCAGATCAGCAACAACCGCAAGTGCTCCAGCCCCAGG |
| | AGTTCTGACACTGAGGACGTGACCAAGAGGAAGAACCATAACTTCTTGGAA |
| | CGAAAAAGGAGGAATGACCTCCGCTCCCGGTTCCTAGCCCTGCGGGACCAG |
| | GTTC (SEQ ID NO: 145) |
| Ms-ccL-Myc | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMWSGFSAAAKLVSEKLASY |
| | QAARKDSTSLSPARGHSVCSTSSLYLQDLTAAASECIDPSVVFPYPLNDSS |
| | SPKSCTSSDSTAFSPSSDSLLSSESSPRASPEPLVLHEETPPTTSSDSEEE |
| | QEDEEEIDVVSVEKRQTPAKRSESGSSPSRGHSKPPHSPLVLKRCHVSTHQ |
| | HNYAAPPSTRKDYPAAKRAKLDSGRVLKQISNNRKCSSPRSSDTEDVTKRK |
| | NHNFLERKRRNDLRSRFLALRDQVPTLASCSKAPKVVILSKALEYLQALVG |
| | AEKKMATEKRQLRCRQQQLQKRIAYLSGY (SEQ ID NO: 146) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGTGG |
| | AGCGGTTTCTCAGCCGCTGCCAAGCTGGTCTCGGAGAAGCTGGCCTCCTAC |
| | CAGGCTGCGCGCAAAGACAGCACCAGCCTGAGCCCCGCCCGCGGGCACAGC |
| | GTCTGCTCCACCTCCAGCCTGTACCTGCAGGACCTCACCGCCGCCGCGTCC |
| | GAGTGCATTGACCCCTCAGTGGTCTTTCCCTACCCGCTCAACGACAGCAGC |
| | TCGCCCAAATCCTGTACCTCGTCCGATTCCACGGCCTTCTCTCCTTCCTCG |
| | GACTCGCTGCTGTCCTCCGAGTCCTCCCCACGGGCCAGCCCTGAGCCCCTA |
| | GTGCTGCATGAGGAGACACCGCCCACCACCAGCAGCGACTCTGAAGAAGAG |
| | CAAGAAGATGAGGAAGAAATTGATGTGGTGTCTGTGGAGAAGAGGCAAACC |
| | CCTGCCAAGAGGTCGGAGTCGGGCTCATCTCCATCCCGAGGCCACAGCAAA |
| | CCTCCGCACAGCCCACTGGTCCTCAAGAGGTGCCACGTCTCCACTCACCAG |
| | CACAACTACGCCGCACCCCCCTCCACAAGGAAGGACTATCCAGCTGCCAAG |
| | AGGGCCAAGTTGGACAGTGGCAGGGTCCTGAAGCAGATCAGCAACAACCGC |
| | AAGT (SEQ ID NO: 147) |
| Ms-cLL-Myc | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMWSGFSAAAKLERVVSDRL |
| | APGAPRGNPPKAPATPDGTPSLEASNPAPATQCQLGEPKTQACSGSESPSD |
| | SEGEEIDVVTVEKRRSLDIRKPVTITVRADPLDPCMKHFHISIHQQQHNYA |
| | ARFPPESCSQEGDPEPGPQEEAPEIEAPKEKEEEEEEEEEEEIVSPPPVGS |
| | EAPQSCHPKPVSSDTEDVTKRKNHNFLERKRRNDLRSRFLALRDQVPTLAS |
| | CSKAPKVVILSKALEYLQALVGAEKKMATEKRQLRCRQQQLQKRIAYLBGY |
| | (SEQ ID NO: 148) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGTGG |
| | AGCGGTTTCTCAGCCGCTGCCAAGCTGGAGAGAGTGGTGAGCGACAGGCTG |
| | GCCCCAGGCGCGCCCCGGGGGAACCCGCCCAAAGCGCCCGCTACCCCGGAC |
| | GGCACTCCTAGTCTGGAAGCCAGTAACCCGGCGCCCGCCACCCAATGTCAG |
| | CTGGGCGAGCCCAAGACTCAGGCCTGCTCCGGGTCCGAGAGCCCCAGCGAT |
| | TCTGAAGGTGAAGAGATTGACGTGGTGACCGTGGAGAAGAGGCGATCTCTG |
| | GACATCCGAAAGCCAGTCACCATCACGGTGCGAGCAGACCCCCTGGACCCC |
| | TGCATGAAGCACTTCCATATCTCTATCCACCAACAGCAGCATAACTATGCT |
| | GCCCGTTTTCCTCCAGAAAGTTGCTCTCAAGAGGGGGATCCTGAGCCAGGT |
| | CCCCAGGAAGAGGCTCCGGAGATAGAAGCTCCCAAGGAGAAAGAGGAGGAG |
| | GAAGAGGAAGAGGAGGAAGAAGAGATTGTGAGCCCCCCACCTGTCGGAAGT |
| | GAGGCTCCCCAGTCCTGCCACCCCAAACCTGTCAGTTCTGACACTGAGGAC |
| | GTGACCAAGAGGAAGAACCATAACTTCTTGGAACGAAAAAGGAGGAATGAC |
| | CTCC (SEQ ID NO: 149) |
| Ms-LLc-Myc | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMWSGFSAR |
| | ERLERVVSDRLAPGAPRGNPPKAPATPDGTPSLEASNPAPATQCQLGEPKT |
| | QACSGSESPSDSEGEEIDVVTVEKRRSLDIRKPVTITVRADPLDPCMKHFH |
| | ISIHQQQHNYAARFPPESCSQEGDPEPGPQEEAPEIEAPKEKEEEEEEEEE |
| | EEIVSPPPVGSEAPQSCHPKPVSSDTEENDKRRTHNVLERQRRNELKRSFF |
| | ALRDQIPELENNEKAPKVVILKKATAYILSIQADEHKLTSEKDLLRKRREQ |
| | LKHKLEQLRNSGA (SEQ ID NO: 150) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGTGGAGCGGCTTCTCCGCCCGA |
| | GAACGGCTGGAGAGAGTGGTGAGCGACAGGCTGGCCCCAGGCGCGCCCCGG |
| | GGGAACCCGCCCAAAGCGCCCGCTACCCCGGACGGCACTCCTAGTCTGGAA |
| | GCCAGTAACCCGGCGCCCGCCACCCAATGTCAGCTGGGCGAGCCCAAGACT |
| | CAGGCCTGCTCCGGGTCCGAGAGCCCCAGCGATTCTGAAGGTGAAGAGATT |
| | GACGTGGTGACCGTGGAGAAGAGGCGATCTCTGGACATCCGAAAGCCAGTC |
| | ACCATCACGGTGCGAGCAGACCCCCTGGACCCCTGCATGAAGCACTTCCAT |
| | ATCTCTATCCACCAACAGCAGCATAACTATGCTGCCCGTTTTCCTCCAGAA |
| | AGTTGCTCTCAAGAGGGGGATCCTGAGCCAGGTCCCCAGGAAGAGGCTCCG |
| | GAGATAGAAGCTCCCAAGGAGAAAGAGGAGGAGGAAGAGGAAGAGGAGGAA |
| | GAAGAGATTGTGAGCCCCCCACCTGTCGGAAGTGAGGCTCCCCAGTCCTGC |
| | CACCCCAAACCTGTCTCCTCAGACACGGAGGAAAACGACAAGAGGCGGACA |
| | CACAACGTCTTGGAACGTCAGAGGAGGAACGAGCTGAAGCGCAGCTTTTTT |
| | GCCCTGCGTGACCAGATCCCTGAATTGGAAAACAACGAAAAGGCCCCCAAG |
| | GTAGTGATCCTCAAAAAAGCCACCGCCTACATCCTGTCCATTCAAGCAGAC |
| | GAGC (SEQ ID NO: 151) |
| Ms-Lcc-Myc | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMWSGFSAR |
| | ERLVSEKLASYQAARKDSTSLSPARGHSVCSTSSLYLQDLTAAASECIDPS |
| | VVFPYPLNDSSSPKSCTSSDSTAFSPSSDSLLSSESSPRASPEPLVLHEET |
| | PPTTSSDSEEEQEDEEEIDVVSVEKRQTPAKRSESGSSPSRGHSKPPHSPL |
| | VLKRCHVSTHQHNYAAPPSTRKDYPAAKRAKLDSGRVLKQISNNRKCSSPR |
| | SSDTEENDKRRTHNVLERQRRNELKRSFFALRDQIPELENNEKAPKVVILK |
| | KATAYILSIQADEHKLTSEKDLLRKRREQLKHKLEQLRNSGA (SEQ ID |
| | NO: 152) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGTGGAGCGGCTTCTCCGCCCGA |
| | GAACGGCTGGTCTCGGAGAAGCTGGCCTCCTACCAGGCTGCGCGCAAAGAC |
| | AGCACCAGCCTGAGCCCCGCCCGCGGGCACAGCGTCTGCTCCACCTCCAGC |
| | CTGTACCTGCAGGACCTCACCGCCGCCGCGTCCGAGTGCATTGACCCCTCA |
| | GTGGTCTTTCCCTACCCGCTCAACGACAGCAGCTCGCCCAAATCCTGTACC |
| | TCGTCCGATTCCACGGCCTTCTCTCCTTCCTCGGACTCGCTGCTGTCCTCC |
| | GAGTCCTCCCCACGGGCCAGCCCTGAGCCCCTAGTGCTGCATGAGGAGACA |
| | CCGCCCACCACCAGCAGCGACTCTGAAGAAGAGCAAGAAGATGAGGAAGAA |
| | ATTGATGTGGTGTCTGTGGAGAAGAGGCAAACCCCTGCCAAGAGGTCGGAG |
| | TCGGGCTCATCTCCATCCCGAGGCCACAGCAAACCTCCGCACAGCCCACTG |
| | GTCCTCAAGAGGTGCCACGTCTCCACTCACCAGCACAACTACGCCGCACCC |
| | CCCTCCACAAGGAAGGACTATCCAGCTGCCAAGAGGGCCAAGTTGGACAGT |
| | GGCAGGGTCCTGAAGCAGATCAGCAACAACCGCAAGTGCTCCAGCCCCAGG |
| | TCCTCAGACACGGAGGAAAACGACAAGAGGCGGACACACAACGTCTTGGAA |
| | CGTCAGAGGAGGAACGAGCTGAAGCGCAGCTTTTTTGCCCTGCGTGACCAG |
| | ATCC (SEQ ID NO: 153) |
| c-MycW135E | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMESGFSAAAKLVSEKLASY |
| | QAARKDSTSLSPARGHSVCSTSSLYLQDLTAAASECIDPSVVFPYPLNDSS |
| | SPKSCTSSDSTAFSPSSDSLLSSESSPRASPEPLVLHEETPPTTSSDSEEE |
| | QEDEEEIDVVSVEKRQTPAKRSESGSSPSRGHSKPPHSPLVLKRCHVSTHQ |
| | HNYAAPPSTRKDYPAAKRAKLDSGRVLKQISNNRKCSSPRSSDTEENDKRR |
| | THNVLERQRRNELKRSFFALRDQIPELENNEKAPKVVILKKATAYILSIQA |
| | DEHKLTSEKDLLRKRREQLKHKLEQLRNSGA (SEQ ID NO: 154) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGGAG |
| | AGCGGTTTCTCAGCCGCTGCCAAGCTGGTCTCGGAGAAGCTGGCCTCCTAC |
| | CAGGCTGCGCGCAAAGACAGCACCAGCCTGAGCCCCGCCCGCGGGCACAGC |
| | GTCTGCTCCACCTCCAGCCTGTACCTGCAGGACCTCACCGCCGCCGCGTCC |
| | GAGTGCATTGACCCCTCAGTGGTCTTTCCCTACCCGCTCAACGACAGCAGC |
| | TCGCCCAAATCCTGTACCTCGTCCGATTCCACGGCCTTCTCTCCTTCCTCG |
| | GACTCGCTGCTGTCCTCCGAGTCCTCCCCACGGGCCAGCCCTGAGCCCCTA |
| | GTGCTGCATGAGGAGACACCGCCCACCACCAGCAGCGACTCTGAAGAAGAG |
| | CAAGAAGATGAGGAAGAAATTGATGTGGTGTCTGTGGAGAAGAGGCAAACC |
| | CCTGCCAAGAGGTCGGAGTCGGGCTCATCTCCATCCCGAGGCCACAGCAAA |
| | CCTCCGCACAGCCCACTGGTCCTCAAGAGGTGCCACGTCTCCACTCACCAG |
| | CACAACTACGCCGCACCCCCCTCCACAAGGAAGGACTATCCAGCTGCCAAG |
| | AGGGCCAAGTTGGACAGTGGCAGGGTCCTGAAGCAGATCAGCAACAACCGC |
| | AAGT (SEQ ID NO: 155) |
| c-MycV394D | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMWSGFSAAAKLVSEKLASY |
| | QAARKDSTSLSPARGHSVCSTSSLYLQDLTAAASECIDPSVVFPYPLNDSS |
| | SPKSCTSSDSTAFSPSSDSLLSSESSPRASPEPLVLHEETPPTTSSDSEEE |
| | QEDEEEIDVVSVEKRQTPAKRSESGSSPSRGHSKPPHSPLVLKRCHVSTHQ |
| | HNYAAPPSTRKDYPAAKRAKLDSGRVLKQISNNRKCSSPRSSDTEENDKRR |
| | THNVLERQRRNELKRSFFALRDQIPELENNEKAPKVDILKKATAYILSIQA |
| | DEHKLTSEKDLLRKRREQLKHKLEQLRNSGA (SEQ ID NO: 156) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGTGG |
| | AGCGGTTTCTCAGCCGCTGCCAAGCTGGTCTCGGAGAAGCTGGCCTCCTAC |
| | CAGGCTGCGCGCAAAGACAGCACCAGCCTGAGCCCCGCCCGCGGGCACAGC |
| | GTCTGCTCCACCTCCAGCCTGTACCTGCAGGACCTCACCGCCGCCGCGTCC |
| | GAGTGCATTGACCCCTCAGTGGTCTTTCCCTACCCGCTCAACGACAGCAGC |
| | TCGCCCAAATCCTGTACCTCGTCCGATTCCACGGCCTTCTCTCCTTCCTCG |
| | GACTCGCTGCTGTCCTCCGAGTCCTCCCCACGGGCCAGCCCTGAGCCCCTA |
| | GTGCTGCATGAGGAGACACCGCCCACCACCAGCAGCGACTCTGAAGAAGAG |
| | CAAGAAGATGAGGAAGAAATTGATGTGGTGTCTGTGGAGAAGAGGCAAACC |
| | CCTGCCAAGAGGTCGGAGTCGGGCTCATCTCCATCCCGAGGCCACAGCAAA |
| | CCTCCGCACAGCCCACTGGTCCTCAAGAGGTGCCACGTCTCCACTCACCAG |
| | CACAACTACGCCGCACCCCCCTCCACAAGGAAGGACTATCCAGCTGCCAAG |
| | AGGGCCAAGTTGGACAGTGGCAGGGTCCTGAAGCAGATCAGCAACAACCGC |
| | AAGT (SEQ ID NO: 157) |
| c-MycL420P | MPLNVNFTNRNYDLDYDSVQPYFICDEEENFYHQQQQSELQPPAPSEDIWK |
| | KFELLPTPPLSPSRRSGLCSPSYVAVATSFSPREDDDGGGGNFSTADQLEM |
| | MTELLGGDMVNQSFICDPDDETFIKNIIIQDCMWSGFSAAAKLVSEKLASY |
| | QAARKDSTSLSPARGHSVCSTSSLYLQDLTAAASECIDPSVVFPYPLNDSS |
| | SPKSCTSSDSTAFSPSSDSLLSSESSPRASPEPLVLHEETPPTTSSDSEEE |
| | QEDEEEIDVVSVEKRQTPAKRSESGSSPSRGHSKPPHSPLVLKRCHVSTHQ |
| | HNYAAPPSTRKDYPAAKRAKLDSGRVLKQISNNRKCSSPRSSDTEENDKRR |
| | THNVLERQRRNELKRSFFALRDQIPELENNEKAPKVVILKKATAYILSIQA |
| | DEHKLTSEKDLPRKRREQLKHKLEQLRNSGA (SEQ ID NO: 158) |
| | ATGCCCCTCAACGTGAACTTCACCAACAGGAACTATGACCTCGACTACGAC |
| | TCCGTACAGCCCTATTTCATCTGCGACGAGGAAGAGAATTTCTATCACCAG |
| | CAACAGCAGAGCGAGCTGCAGCCGCCCGCGCCCAGTGAGGATATCTGGAAG |
| | AAATTCGAGCTGCTTCCCACCCCGCCCCTGTCCCCGAGCCGCCGCTCCGGG |
| | CTCTGCTCTCCATCCTATGTTGCGGTCGCTACGTCCTTCTCCCCAAGGGAA |
| | GACGATGACGGCGGCGGTGGCAACTTCTCCACCGCCGATCAGCTGGAGATG |
| | ATGACCGAGTTACTTGGAGGAGACATGGTGAACCAGAGCTTCATCTGCGAT |
| | CCTGACGACGAGACCTTCATCAAGAACATCATCATCCAGGACTGTATGTGG |
| | AGCGGTTTCTCAGCCGCTGCCAAGCTGGTCTCGGAGAAGCTGGCCTCCTAC |
| | CAGGCTGCGCGCAAAGACAGCACCAGCCTGAGCCCCGCCCGCGGGCACAGC |
| | GTCTGCTCCACCTCCAGCCTGTACCTGCAGGACCTCACCGCCGCCGCGTCC |
| | GAGTGCATTGACCCCTCAGTGGTCTTTCCCTACCCGCTCAACGACAGCAGC |
| | TCGCCCAAATCCTGTACCTCGTCCGATTCCACGGCCTTCTCTCCTTCCTCG |
| | GACTCGCTGCTGTCCTCCGAGTCCTCCCCACGGGCCAGCCCTGAGCCCCTA |
| | GTGCTGCATGAGGAGACACCGCCCACCACCAGCAGCGACTCTGAAGAAGAG |
| | CAAGAAGATGAGGAAGAAATTGATGTGGTGTCTGTGGAGAAGAGGCAAACC |
| | CCTGCCAAGAGGTCGGAGTCGGGCTCATCTCCATCCCGAGGCCACAGCAAA |
| | CCTCCGCACAGCCCACTGGTCCTCAAGAGGTGCCACGTCTCCACTCACCAG |
| | CACAACTACGCCGCACCCCCCTCCACAAGGAAGGACTATCCAGCTGCCAAG |
| | AGGGCCAAGTTGGACAGTGGCAGGGTCCTGAAGCAGATCAGCAACAACCGC |
| | AAGT (SEQ ID NO: 159) |
| L-MycW96E | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMESGFSAR |
| | ERLERVVSDRLAPGAPRGNPPKAPATPDGTPSLEASNPAPATQCQLGEPKT |
| | QACSGSESPSDSEGEEIDVVTVEKRRSLDIRKPVTITVRADPLDPCMKHFH |
| | ISIHQQQHNYAARFPPESCSQEGDPEPGPQEEAPEIEAPKEKEEEEEEEEE |
| | EEIVSPPPVGSEAPQSCHPKPVSSDTEDVTKRKNHNFLERKRRNDLRSRFL |
| | ALRDQVPTLASCSKAPKVVILSKALEYLQALVGAEKKMATEKRQLRCRQQQ |
| | LQKRIAYLSGY (SEQ ID NO: 160) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGGAGAGCGGCTTCTCCGCCCGA |
| | GAACGGCTGGAGAGAGTGGTGAGCGACAGGCTGGCCCCAGGCGCGCCCCGG |
| | GGGAACCCGCCCAAAGCGCCCGCTACCCCGGACGGCACTCCTAGTCTGGAA |
| | GCCAGTAACCCGGCGCCCGCCACCCAATGTCAGCTGGGCGAGCCCAAGACT |
| | CAGGCCTGCTCCGGGTCCGAGAGCCCCAGCGATTCTGAAGGTGAAGAGATT |
| | GACGTGGTGACCGTGGAGAAGAGGCGATCTCTGGACATCCGAAAGCCAGTC |
| | ACCATCACGGTGCGAGCAGACCCCCTGGACCCCTGCATGAAGCACTTCCAT |
| | ATCTCTATCCACCAACAGCAGCATAACTATGCTGCCCGTTTTCCTCCAGAA |
| | AGTTGCTCTCAAGAGGGGGATCCTGAGCCAGGTCCCCAGGAAGAGGCTCCG |
| | GAGATAGAAGCTCCCAAGGAGAAAGAGGAGGAGGAAGAGGAAGAGGAGGAA |
| | GAAGAGATTGTGAGCCCCCCACCTGTCGGAAGTGAGGCTCCCCAGTCCTGC |
| | CACCCCAAACCTGTCAGTTCTGACACTGAGGACGTGACCAAGAGGAAGAAC |
| | CATAACTTCTTGGAACGAAAAAGGAGGAATGACCTCCGCTCCCGGTTCCTA |
| | GCCCTGCGGGACCAGGTTCCCACCCTGGCCAGCTGCTCTAAGGCCCCCAAA |
| | GTCGTGATCCTCAGCAAGGCGTTAGAATACTTGCAGGCTTTGGTGGGGGCT |
| | GAAA (SEQ ID NO: 161) |
| L-MycV325D | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMWSGFSAR |
| | ERLERVVSDRLAPGAPRGNPPKAPATPDGTPSLEASNPAPATQCQLGEPKT |
| | QACSGSESPSDSEGEEIDVVTVEKRRSLDIRKPVTITVRADPLDPCMKHFH |
| | ISIHQQQHNYAARFPPESCSQEGDPEPGPQEEAPEIEAPKEKEEEEEEEEE |
| | EEIVSPPPVGSEAPQSCHPKPVSSDTEDVTKRKNHNFLERKRRNDLRSRFL |
| | ALRDQVPTLASCSKAPKVDILSKALEYLQALVGAEKKMATEKRQLRCRQQQ |
| | LQKRIAYLSGY (SEQ ID NO: 162) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGTGGAGCGGCTTCTCCGCCCGA |
| | GAACGGCTGGAGAGAGTGGTGAGCGACAGGCTGGCCCCAGGCGCGCCCCGG |
| | GGGAACCCGCCCAAAGCGCCCGCTACCCCGGACGGCACTCCTAGTCTGGAA |
| | GCCAGTAACCCGGCGCCCGCCACCCAATGTCAGCTGGGCGAGCCCAAGACT |
| | CAGGCCTGCTCCGGGTCCGAGAGCCCCAGCGATTCTGAAGGTGAAGAGATT |
| | GACGTGGTGACCGTGGAGAAGAGGCGATCTCTGGACATCCGAAAGCCAGTC |
| | ACCATCACGGTGCGAGCAGACCCCCTGGACCCCTGCATGAAGCACTTCCAT |
| | ATCTCTATCCACCAACAGCAGCATAACTATGCTGCCCGTTTTCCTCCAGAA |
| | AGTTGCTCTCAAGAGGGGGATCCTGAGCCAGGTCCCCAGGAAGAGGCTCCG |
| | GAGATAGAAGCTCCCAAGGAGAAAGAGGAGGAGGAAGAGGAAGAGGAGGAA |
| | GAAGAGATTGTGAGCCCCCCACCTGTCGGAAGTGAGGCTCCCCAGTCCTGC |
| | CACCCCAAACCTGTCAGTTCTGACACTGAGGACGTGACCAAGAGGAAGAAC |
| | CATAACTTCTTGGAACGAAAAAGGAGGAATGACCTCCGCTCCCGGTTCCTA |
| | GCCCTGCGGGACCAGGTTCCCACCCTGGCCAGCTGCTCTAAGGCCCCCAAA |
| | GTCGACATCCTCAGCAAGGCGTTAGAATACTTGCAGGCTTTGGTGGGGGCT |
| | GAAA (SEQ ID NO: 163) |
| L-MycL351P | MDFDSYQHYFYDYDCGEDFYRSTAPSEDIWKKFELVPSPPTSPPWGSGPGA |
| | VDPASGINPGEPWPGGGAGDEAESRGHSKAWGRNYASIIRRDCMWSGFSAR |
| | ERLERVVSDRLAPGAPRGNPPKAPATPDGTPSLEASNPAPATQCQLGEPKT |
| | QACSGSESPSDSEGEEIDVVTVEKRRSLDIRKPVTITVRADPLDPCMKHFH |
| | ISIHQQQHNYAARFPPESCSQEGDPEPGPQEEAPEIEAPKEKEEEEEEEEE |
| | EEIVSPPPVGSEAPQSCHPKPVSSDTEDVTKRKNHNFLERKRRNDLRSRFL |
| | ALRDQVPTLASCSKAPKVVILSKALEYLQALVGAEKKMATEKRQPRCRQQQ |
| | LQKRIAYLSGY (SEQ ID NO: 164) |
| | ATGGACTTCGACTCGTATCAGCACTATTTCTACGACTATGACTGCGGAGAG |
| | GATTTCTACCGCTCCACGGCGCCCAGCGAGGACATCTGGAAGAAATTCGAG |
| | CTGGTGCCGTCGCCCCCCACGTCGCCGCCCTGGGGCTCCGGTCCCGGCGCC |
| | GTGGACCCAGCCTCTGGGATTAATCCCGGGGAGCCGTGGCCTGGAGGGGGT |
| | GCCGGGGACGAGGCGGAATCTCGGGGCCATTCGAAAGCCTGGGGCAGGAAT |
| | TATGCTTCCATCATTCGCCGTGACTGCATGTGGAGCGGCTTCTCCGCCCGA |
| | GAACGGCTGGAGAGAGTGGTGAGCGACAGGCTGGCCCCAGGCGCGCCCCGG |
| | GGGAACCCGCCCAAAGCGCCCGCTACCCCGGACGGCACTCCTAGTCTGGAA |
| | GCCAGTAACCCGGCGCCCGCCACCCAATGTCAGCTGGGCGAGCCCAAGACT |
| | CAGGCCTGCTCCGGGTCCGAGAGCCCCAGCGATTCTGAAGGTGAAGAGATT |
| | GACGTGGTGACCGTGGAGAAGAGGCGATCTCTGGACATCCGAAAGCCAGTC |
| | ACCATCACGGTGCGAGCAGACCCCCTGGACCCCTGCATGAAGCACTTCCAT |
| | ATCTCTATCCACCAACAGCAGCATAACTATGCTGCCCGTTTTCCTCCAGAA |
| | AGTTGCTCTCAAGAGGGGGATCCTGAGCCAGGTCCCCAGGAAGAGGCTCCG |
| | GAGATAGAAGCTCCCAAGGAGAAAGAGGAGGAGGAAGAGGAAGAGGAGGAA |
| | GAAGAGATTGTGAGCCCCCCACCTGTCGGAAGTGAGGCTCCCCAGTCCTGC |
| | CACCCCAAACCTGTCAGTTCTGACACTGAGGACGTGACCAAGAGGAAGAAC |
| | CATAACTTCTTGGAACGAAAAAGGAGGAATGACCTCCGCTCCCGGTTCCTA |
| | GCCCTGCGGGACCAGGTTCCCACCCTGGCCAGCTGCTCTAAGGCCCCCAAA |
| | GTCGTGATCCTCAGCAAGGCGTTAGAATACTTGCAGGCTTTGGTGGGGGCT |
| | GAAA (SEQ ID NO: 165) |

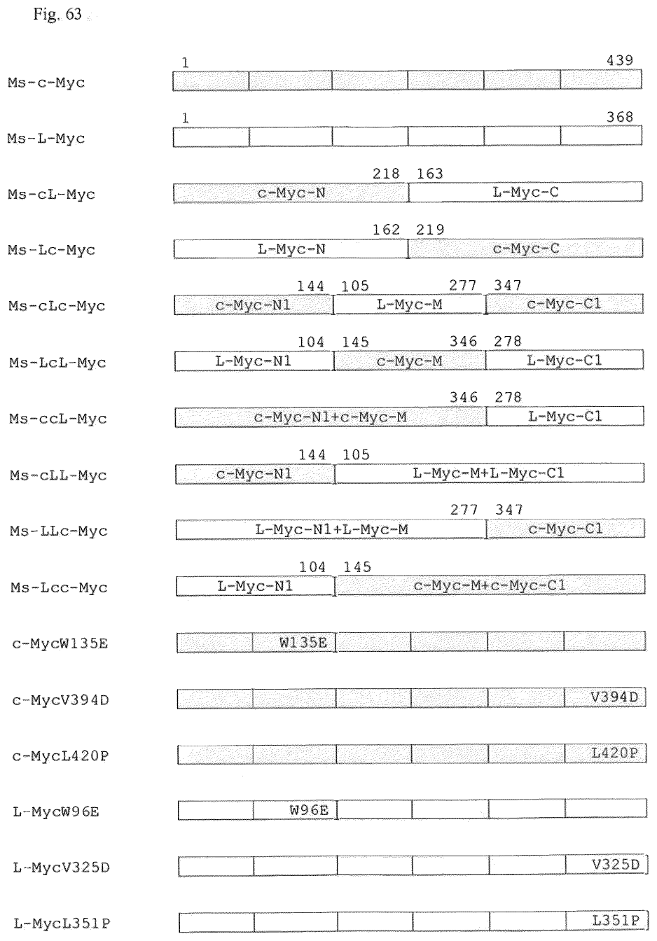

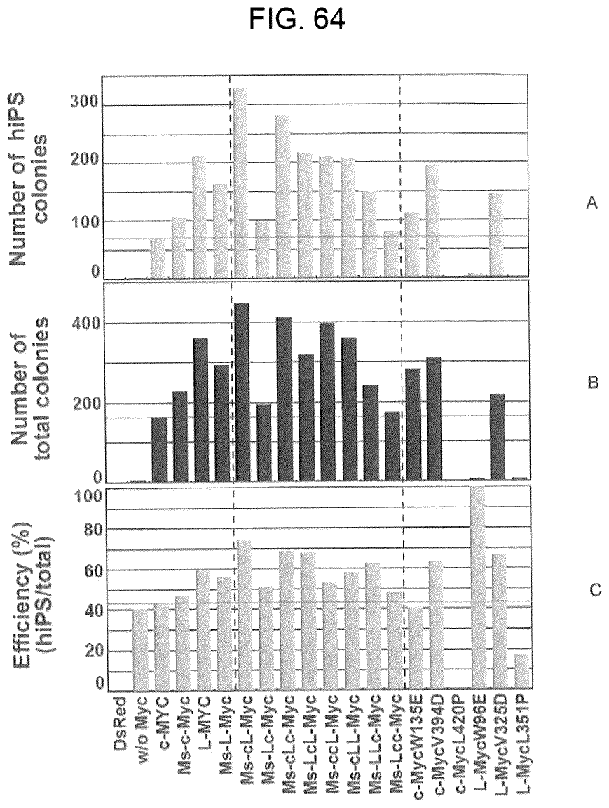

The Myc chimeras and variants were constructed as described below (see also FIG. 63). At first, mouse c-Myc (Ms-c-Myc) and L-Myc (Ms-L-Myc) were respectively inserted into pENTR-D-TOPO (Invitrogen) to prepare pENTR-D-TOPO-Ms-c-Myc and pENTR-O-TOPO-Ms-L-Myc. Then, primers for introducing a point mutation shown in FIG. 63 were prepared based on the sequence information of mouse c-Myc (NCBI Ace. No. NM_010849) and L-Myc (NCBI Ace. No. NM_008506), and PCR was carried out with these primers using pENTR-D-TOPO-Ms-c-Myc and pENTR-D-TOPO-Ms-LMyc as a template. After confirmation of each mutation by sequencing, the PCR product was subcloned into a retrovirus vector pMXs by LR reaction. Each fragment of chimera Myc shown in FIG. 63 was amplified by PCR. Then, PCR was carried out using a mixture of combinations of each fragment as a template. The obtained PCR product was subcloned into a retrovirus vector pMXs as mentioned above. The thus obtained each retrovirus, and each retrovirus vector of SOX2, OCT3/4, and KLF4 used in Example 28 were transduced into adult human dermal fibroblast (aHDF-Slc7a1) in the same way as in Example 28. On the 31^{st} day after retrovirus infection, the number of total colonies and the number of human iPS colonies (hiPS colony) which appeared were counted. The results are shown in TABLE 22 and FIGS. 64A-C. FIGS. 64A-C are graphs of the results presented in TABLE 22.

TABLE 22
Summary of hiPS Cell Induction with Chimeric and Mutant Myc

| | | | | | | hiPS | Total | |
|---|---|---|---|---|---|---|---|---|
| | | | | | | colonies | colonies | hiPS (%) |
| 1 | Neck | CCT4 | Mock | Mock | A | 0 | 0 | 0 |
| 2 | SOX2 | CCT4 | KLF4 | -MYC | B | 2 | 5 | 40 |
| 3 | SOX2 | CCT4 | KLF4 | c-MYC | C | 70 | 162 | 43.2 |
| 4 | SOX2 | CCT4 | KLF4 | Ms-c-Myc | D | 106 | 228 | 46.5 |
| 5 | SOX2 | CCT4 | KLF4 | L-MYC | E | 212 | 358 | 59.2 |
| 6 | SOX2 | CCT4 | KLF4 | Ms-L-Myc | F | 164 | 292 | 56.2 |
| 13 | SOX2 | CCT4 | KLF4 | Ms-cL-Myc | M | 330 | 446 | 74.0 |
| 14 | SOX2 | CCT4 | KLF4 | Ms-Lc-Myc | N | 98 | 192 | 51.0 |
| 15 | SOX2 | CCT4 | KLF4 | Ms-cLc-Myc | O | 282 | 410 | 68.8 |
| 16 | SOX2 | CCT4 | KLF4 | Ms-LcL-Myc | P | 216 | 318 | 67.9 |
| 17 | SOX2 | CCT4 | KLF4 | MS-ccL-Myc | Q | 209 | 394 | 53.0 |
| 18 | SOX2 | CCT4 | KLF4 | Ms-cLL-Myc | R | 208 | 359 | 57.9 |
| 19 | SOX2 | CCT4 | KLF4 | Ms-LLC-Myc | S | 150 | 240 | 62.5 |
| 20 | SOX2 | CCT4 | KLF4 | Ms-Lcc-Myc | T | 81 | 170 | 47.6 |
| 7 | SOX2 | CCT4 | KLF4 | Ms-c-Myc-W135E | G | 112 | 280 | 40.0 |
| 8 | SOX2 | CCT4 | KLF4 | Ms-c-Myc-V394D | H | 194 | 308 | 63.0 |
| 9 | SOX2 | CCT4 | KLF4 | Ms-c-Myc-L420D | I | 0 | 0 | 0.0 |
| 10 | SOX2 | CCT4 | KLF4 | Ms-L-Myc-W96E | J | 6 | 6 | 100.0 |
| 11 | SOX2 | CCT4 | KLF4 | Ms-L-Myc-V325D | K | 144 | 216 | 66.7 |
| 12 | SOX2 | CCT4 | KLF4 | Ms-L-Nyc-L351P | L | 1 | 6 | 16.7 |

c-Myc and L-Myc have approximately 100% identity at the amino acid level between human and mouse. As shown in FIGS. 64A-C and TABLE 22, human iPS colonies were generated when mouse genes (Ms-c-Myc and Ms-L-Myc) were used, in the same way as in the case where human genes (c-MYC and L-MYC) were used. Therefore, it was confirmed that mouse c-Myc and L-Myc had the same function as that of human c-MYC and L-MYC.

The results obtained by using various Myc chimera genes and point-mutated genes were compared with those obtained by using wild-type Myc. As a result, it was found that the iPS colony number was increased when Ms-cL-Myc or Ms-cLc-Myc was used, as compared with the wild-type L-Myc (L-MYC), and that the best result was obtained when Ms-cL-Myc was used. Also, from the result obtained by using the point-mutated genes, it was revealed that one amino acid at the C-terminus (c-MycL420P and L-MycL351P) of c-Myc and L-Myc were very important. From the aforementioned results, it was suggested that the functional regions of Myc which were important for establishment of human iPS cells included the N-terminal third of c-Myc, the central region of L-Myc, and the C-terminal region of c/L-Myc.

### Kits of the Present Invention.

One aspect of the present invention includes kits designed for use in the preparation and induction of iPS cells. Another aspect of the invention comprises kits for the prevention or treatment of a medical condition or disease through the use of an NRF, an iPS cell or a cell derived from an iPS cell by induction of differentiation. It is also an object of the present invention to provide compositions and methods useful for in vitro transfection, in vivo transfection, ex vivo transfection, in situ labeling, diagnostic tests, genetic therapy, gene therapy, treatment of medical conditions, and the creation of transgenic animals. One aspect of the present invention comprises kits designed for in vitro transfection, in vivo transfection, ex vivo transfection, in situ labeling, diagnostic tests, genetic therapy, gene therapy, treatment of medical conditions, and the creation of transgenic animals. Accordingly, the present invention includes a composition comprising one or more NRFs, one or more iPS cells, one or more cells derived from an iPS cell or cells, and combinations thereof.

### Utility and Practical Applications

By using the nuclear reprogramming factor provided by the present invention, reprogramming of differentiated cell nuclei can be conveniently and highly reproducibly induced without using embryos or ES cells, and induced pluripotent stem cells as undifferentiated cells having differentiation ability, pluripotency and growth ability similar to those of ES cells can be established.

Uses of the induced pluripotent stem cells prepared by the method of the present invention are not particularly limited. The cells can be used for any experiments and research conducted with ES cells, therapeutic treatments utilizing ES cells and the like. For example, desired differentiated cells (e.g., nerve cells, cardiac muscle cells, hemocyte cells and the like) can be derived by treating induced pluripotent stem cells obtained by the method of the present invention with retinoic acid, growth factors such as EGF, glucocorticoid or the like, and stem cell therapy based on cellular auto-transplantation can be achieved by returning the differentiated cells obtained as described above to the patient. However, uses of the induced pluripotent stem cells of the present invention are not limited to the aforementioned specific embodiments.

Thus, the present invention has enabled the generation of iPS cells from adult human dermal fibroblasts and other human somatic cells, which are indistinguishable from human ES cells in their differentiation potential in vitro and in teratomas. Furthermore, the instant invention allows for the generation of patient- and disease-specific pluripotent stem cells. Even with the presence of retroviral integration, human iPS cells are useful for understanding disease mechanisms, drug screening, and toxicology. For example, hepatocytes derived from iPS cells with various genetic and disease backgrounds can be utilized in predicting liver toxicity of drug candidates. Human iPS cells may overcome the ethical issues that hES cells confront.

Reference is made to the following documents: Takahashi, et al. Cell 131:861-872, 2007; Nakagawa et al. Nature Biotechnology 26(1): 101-106, 2008; Takahashi et al. Cell 126:663-676, 2006; Okita et al. Nature 448:313-17, 2007; Takahashi et al. Nature Protocols 2(12): 3081-89, 2007; and the supplementary figures and data associated with these documents, all of which are incorporated by reference herein in their entireties.

Although the present invention has been described in considerable detail with regard to certain versions thereof, other versions are possible, and alterations, permutations, and equivalents of the versions shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

The attached Sequence Listing includes those sequences disclosed in U.S. patent application Ser. No. 12/213,035, which is incorporated by reference herein in its entirety.

1. A method for preparing an induced pluripotent stem cell from a somatic cell, comprising introducing a nuclear reprogramming factor into a somatic cell, wherein said method comprises a step of selecting a nuclear reprogramming factor comprising a plurality of genes selected from the following 24 candidates of genes relevant to nuclear reprogramming: ECAT1, ECAT2, ECAT3, ECAT4, ECAT5, ECAT7, ECAT8, ECAT9, ECAT10, ECAT15-1, ECAT15-2, Fth117, Sal14, Oct3/4, Sox2, Rex1, Utf1, Tel1, Stella, KLf4, ß-catenin, c-Myc, Stat3, and Grb2; and wherein by using the nuclear reprogramming factor, the nucleus of a somatic cell can be reprogrammed to obtain an induced pluripotent stem cell.

2. The method of claim 1, wherein the nuclear reprogramming factor comprises gene products of the following total of ten kinds of genes: ECAT3, ECAT4, ECAT5, ECAT15-2, Oct3/4, Sox2, Tel1, KLf4, ß-catenin, and c-Myc.

3. A method for preparing a somatic cell, comprising preparing an induced pluripotent stem cell by the method according to claim 1, and differentiating the induced pluripotent stem cell into the somatic cell.
